(19) United States
(12) Patent Application Publication
(10) Pub. No.: US 2025/0228126 A1
(43) Pub. Date: Jul. 10, 2025

(54) COMPOUND, FUNCTIONAL MATERIAL, ELECTRONIC ELEMENT AND ELECTRONIC DEVICE

(71) Applicant: GUANGDONG AGLAIA OPTOELECTRONIC MATERIALS CO., LTD, Guangdong (CN)

(72) Inventors: Shaofu CHEN, Guangdong (CN); Xu WANG, Guangdong (CN); Kangzhi YE, Guangdong (CN); Lei DAI, Guangdong (CN); Lifei CAI, Guangdong (CN)

(73) Assignee: GUANGDONG AGLAIA OPTOELECTRONIC MATERIALS CO., LTD, Guangdong (CN)

(21) Appl. No.: 19/012,646

(22) Filed: Jan. 7, 2025

(30) Foreign Application Priority Data
Jan. 8, 2024 (CN) 202410023251.5

(51) Int. Cl. H10K 85/60; C07D 213/16; C07D 239/26; C07D 251/24; C07D 401/10; C07D 401/14; C07D 405/04; C07D 405/10; C07D 409/04; C07D 409/10; H10K 50/16

(52) U.S. Cl. CPC H10K 85/615; C07D 213/16; C07D 239/26; C07D 251/24; C07D 401/10; C07D 401/14; C07D 405/04; C07D 405/10; C07D 409/04; C07D 409/10; H10K 85/626; H10K 85/654; H10K 85/6574; H10K 50/16

(57) ABSTRACT

The present disclosure belongs to the technical field of organic optoelectronic materials, and relates to a compound, a functional material, an electronic element and an electronic device. The compound has the following structural formula, where ring A is an aliphatic ring, and the number of ring carbon atoms in the aliphatic ring is e; and R₄ is selected from deuterium or R₈ group substituted by deuterium. The compound of the present disclosure is formed by connecting deuterated alkyl fluorene and nitrogen heterocycle, has many advantages such as good film-forming property, good optical, electrical and thermal stability, high luminous efficiency, low voltage, long lifetime, etc., and can be used in organic light-emitting devices, especially as hole blocking layer materials or electron transport material layers, and has potential application prospects in many industries such as AMOLED.

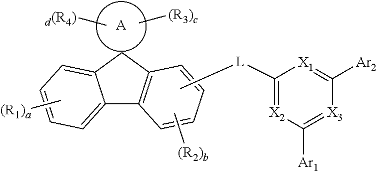

### CROSS-REFERENCE TO RELATED APPLICATION

This application claims priority to Chinese Patent Application No. 202410023251.5, filed Jan. 8, 2024, the disclosure of which is hereby incorporated by reference in its entirety.

### BACKGROUND

### Technical Field

The present disclosure belongs to the technical field of organic optoelectronic materials, and in particular, to a compound, a functional material, an electronic element and an electronic device.

### TECHNICAL CONSIDERATIONS

At present, as a new generation of display technology, organic light-emitting diode (OLED) devices have received more and more attention in both display and lighting technology, and have broad application prospects. However, compared with market application requirements, the luminous efficiency, driving voltage, service life and other properties of OLED devices in related technologies need to be further strengthened and improved.

Generally speaking, the basic structure of an OLED device consists of metal electrodes (a cathode and an anode) and organic functional material thin film layers sandwiched therebetween. The thin film layers contain various organic functional material layers with different functions, just like a sandwich structure. Driven by current, holes and electrons are injected from the anode and cathode, respectively. After moving a certain distance, the holes and electrons are recombined in the light-emitting layer and released in the form of light or heat, thus resulting in light emission of the OLED. However, the organic functional materials are the core components of the OLED devices, and their thermal stability, photochemical stability, electrochemical stability, quantum yield, film formation stability, crystallinity, color saturation and the like are all important factors affecting the performance of the devices.

With the continuous improvement of market requirements for the OLED devices, the development of new organic functional materials has become an urgent problem for those skilled in the art.

### SUMMARY

The present disclosure aims to solve at least one of the technical problems existing in the above-mentioned existing technologies. To this end, a compound with a novel structure is proposed herein, which provides a new direction for improving the performance of the OLED devices.

Use of said compound is further proposed herein.

Specifically, a compound has the following structural formula:

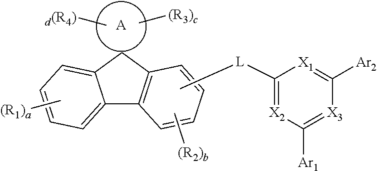

wherein ring is an aliphatic ring, an the number of ring carbon atoms in the aliphatic ring is e;
X₁, X₂ and X₃ are independently selected from N or CR₀, and at least one of the X₁, X₂ and X₃ is N;
R₀ is selected from hydrogen, deuterium, halogen, cyano, nitro, a substituted or unsubstituted aliphatic hydrocarbyl group with a main chain having ≤40 carbon atoms, a substituted or unsubstituted heteroalkyl group with a main chain having 1-40 carbon atoms, a substituted or unsubstituted alkoxy group with a main chain having 1-40 carbon atoms, a substituted or unsubstituted alkylsilyl group with a main chain having 1-40 carbon atoms, a substituted or unsubstituted alkylboryl group with a main chain having 1-40 carbon atoms, a substituted or unsubstituted alicyclic hydrocarbyl group having 3-40 ring-forming carbon atoms, a substituted or unsubstituted heterocycloalkyl group having 3-40 ring-forming carbon atoms, a substituted or unsubstituted aryl group having 6-60 aromatic ring carbon atoms, a substituted or unsubstituted aryloxy group having 6-60 aromatic ring carbon atoms, a substituted or unsubstituted arylsilyl group having 6-60 aromatic ring carbon atoms, a substituted or unsubstituted arylboryl group having 6-60 aromatic ring carbon atoms, a substituted or unsubstituted arylamine group having 6-60 aromatic ring carbon atoms, a substituted or unsubstituted arylphosphine group having 6-60 aromatic ring carbon atoms, or a substituted or unsubstituted heteroaryl group having 3-60 aromatic ring carbon atoms;
R₁, R₂, and R₃ are each independently selected from deuterium, halogen, cyano, nitro, a substituted or unsubstituted aliphatic hydrocarbyl group with a main chain having ≤40 carbon atoms, a substituted or unsubstituted heteroalkyl group with a main chain having 1-40 carbon atoms, a substituted or unsubstituted alkoxy group with a main chain having 1-40 carbon atoms, a substituted or unsubstituted alkylsilyl group with a main chain having 1-40 carbon atoms, a substituted or unsubstituted alkylboryl group with a main chain having 1-40 carbon atoms, a substituted or unsubstituted alicyclic hydrocarbyl group having 3-40 ring-forming carbon atoms, a substituted or unsubstituted heterocycloalkyl group having 3-40 ring-forming carbon atoms, a substituted or unsubstituted aryl group having 6-60 aromatic ring carbon atoms, a substituted or unsubstituted aryloxy group having 6-60 aromatic ring carbon atoms, a substituted or unsubstituted arylsilyl group having 6-60 aromatic ring carbon atoms, a substituted or unsubstituted arylboryl group having 6-60 aromatic ring carbon atoms, a substituted or unsubstituted arylamine group having 6-60 aromatic ring carbon atoms, a substituted or unsubstituted arylphosphine group having 6-60 aromatic ring carbon atoms, or a substituted or unsubstituted heteroaryl group having 3-60 aromatic ring carbon atoms;
R₄ is selected from deuterium or R₈ group substituted by deuterium;
R₈ is selected from a substituted or unsubstituted aliphatic hydrocarbyl group with a main chain having ≤40 carbon atoms, a substituted or unsubstituted heteroalkyl group with a main chain having 1-40 carbon atoms, a substituted or unsubstituted alkoxy group with a main chain having 1-40 carbon atoms, a substituted or unsubstituted alkylsilyl group with a main chain having 1-40 carbon atoms, a substituted or unsubstituted alkylboryl group with a main chain having 1-40 carbon atoms, a substituted or unsubstituted alicyclic hydrocarbyl group having 3-40 ring-forming carbon atoms, a substituted or unsubstituted heterocycloalkyl group having 3-40 ring-forming carbon atoms, a substituted or unsubstituted aryl group having 6-60 aromatic ring carbon atoms, a substituted or unsubstituted aryloxy group having 6-60 aromatic ring carbon atoms, a substituted or unsubstituted arylsilyl group having 6-60 aromatic ring carbon atoms, a substituted or unsubstituted arylboryl group having 6-60 aromatic ring carbon atoms, a substituted or unsubstituted arylamine group having 6-60 aromatic ring carbon atoms, a substituted or unsubstituted arylphosphine group having 6-60 aromatic ring carbon atoms, or a substituted or unsubstituted heteroaryl group having 3-60 aromatic ring carbon atoms.

a is an integer selected from 0-4; if a is an integer≥2, the adjacent R₁ are connected to each other to form a fused ring or not connected.

b is an integer selected from 0 to 3; if b is an integer≥2, the adjacent R₂ are connected to each other to form a fused ring or not connected.

c is selected from an integer≥0.

d is selected from an integer≥1.

L is selected from a single bond, a substituted or unsubstituted arylene group, or a substituted or unsubstituted heteroarylene group.

Ar₁ and Ar₂ are independently selected from a substituted or unsubstituted aryl group, or a substituted or unsubstituted heteroaryl group.

Heteroatoms in the heteroalkyl, heterocycloalkyl, heteroaryl or heteroarylene group are independently selected from at least one of O, S, N, Se, Si and Ge.

The term “substituted” refers to being substituted by at least one of deuterium, halogen, cyano, isocyano, phosphine group, an alkyl group having 1-6 carbon atoms, a cycloalkyl group having 3-16 ring carbon atoms, an alkyl-substituted amine group having 1-6 carbon atoms, an aryl group having 6-12 carbon atoms, or a deuterated aryl group having 6-12 carbon atoms, where the substituted number ranges from a monosubstitution to a maximum substitution.

In some embodiments of the present disclosure, the adjacent R₁ refer to R₁ located on the same carbon atom or adjacent carbon atoms, and the adjacent R₂ refer to R₂ located on the same carbon atom or adjacent carbon atoms.

In some embodiments of the present disclosure, the aliphatic hydrocarbyl group with a main chain having ≤40 carbon atoms includes at least one of an alkyl group with a main chain having 1-40 carbon atoms, an alkenyl group with a main chain having 2-40 carbon atoms, and an alkynyl group with a main chain having 2-40 carbon atoms.

In some embodiments of the present disclosure, the alkyl group having 1-6 carbon atoms includes at least one of methyl, ethyl, propyl, butyl, pentyl, and hexyl.

In some embodiments of the present disclosure, the propyl includes at least one of n-propyl, isopropyl, and tert-propyl.

In some embodiments of the present disclosure, the butyl includes at least one of n-butyl, isobutyl, tert-butyl, and sec-butyl.

In some embodiments of the present disclosure, the pentyl includes at least one of n-pentyl, tert-pentyl, neopentyl and tert-pentyl.

In some embodiments of the present disclosure, the cycloalkyl group having 3-16 ring carbon atoms includes a cycloalkyl group having 3-6 ring carbon atoms.

In some embodiments of the present disclosure, the cycloalkyl group having 3-6 ring carbon atoms is at least one of a cyclopropyl group, a cyclopentyl group, a cyclohexyl group, and an adamantly group.

In some embodiments of the present disclosure, R₀, R₁, R₂, and R₃ are independently selected from a substituted or unsubstituted aliphatic hydrocarbyl group with a main chain having ≤20 carbon atoms, a substituted or unsubstituted heteroalkyl group with a main chain having 1-20 carbon atoms, a substituted or unsubstituted alkoxy group with a main chain having 1-20 carbon atoms, a substituted or unsubstituted alkylsilyl group with a main chain having 1-20 carbon atoms, a substituted or unsubstituted alkylboryl group with a main chain having 1-20 carbon atoms, a substituted or unsubstituted alicyclic hydrocarbyl group having 3-20 ring-forming carbon atoms, a substituted or unsubstituted heterocycloalkyl group having 3-20 ring-forming carbon atoms, a substituted or unsubstituted aryl group having 6-30 aromatic ring carbon atoms, a substituted or unsubstituted aryloxy group having 6-30 aromatic ring carbon atoms, a substituted or unsubstituted arylsilyl group having 6-30 aromatic ring carbon atoms, a substituted or unsubstituted arylboryl group having 6-30 aromatic ring carbon atoms, a substituted or unsubstituted arylamine group having 6-30 aromatic ring carbon atoms, a substituted or unsubstituted arylphosphine group having 6-30 aromatic ring carbon atoms, or a substituted or unsubstituted heteroaryl group having 3-30 aromatic ring carbon atoms.

In some embodiments of the present disclosure, R₀, R₁, R₂, and R₃ are independently selected from a substituted or unsubstituted aliphatic hydrocarbyl group with a main chain having ≤10 carbon atoms, a substituted or unsubstituted heteroalkyl group with a main chain having 1-10 carbon atoms, a substituted or unsubstituted alkoxy group with a main chain having 1-10 carbon atoms, a substituted or unsubstituted alkylsilyl group with a main chain having 1-10 carbon atoms, a substituted or unsubstituted alkylboryl group with a main chain having 1-10 carbon atoms, a substituted or unsubstituted alicyclic hydrocarbyl group having 3-10 ring-forming carbon atoms, a substituted or unsubstituted heterocycloalkyl group having 3-10 ring-forming carbon atoms, a substituted or unsubstituted aryl group having 6-12 aromatic ring carbon atoms, a substituted or unsubstituted aryloxy group having 6-12 aromatic ring carbon atoms, a substituted or unsubstituted arylsilyl group having 6-12 aromatic ring carbon atoms, a substituted or unsubstituted arylboryl group having 6-12 aromatic ring carbon atoms, a substituted or unsubstituted arylamine group having 6-12 aromatic ring carbon atoms, a substituted or unsubstituted arylphosphine group having 6-12 aromatic ring carbon atoms, or a substituted or unsubstituted heteroaryl group having 3-12 aromatic ring carbon atoms.

In some embodiments of the present disclosure, two of X₁, X₂ and X₃ are N, or each of the X₁, X₂ and X₃ are N; R₁ and R₂ are independently selected from deuterium, halogen, cyano, nitro, an alkyl group with a main chain having 1-10 carbon atoms, an alkoxy group with a main chain having 1-10 carbon atoms, a heteroalkyl group with a main chain having 1-10 carbon atoms, a cycloalkyl group having 3-10 ring-forming carbon atoms, a heterocycloalkyl group having 3-10 ring-forming carbon atoms, an aryl group having 6-12 carbon atoms, an aryloxy group having 6-12 carbon atoms, an arylamine group having 6-12 carbon atoms, an arylphosphine group having 6-12 carbon atoms, or a heteroaryl group having 5-20 carbon atoms; and R₃ is selected from halogen, cyano, nitro, an alkyl group with a main chain having 1-10 carbon atoms, an alkoxy group with a main chain having 1-10 carbon atoms, a heteroalkyl group with a main chain having 1-10 carbon atoms, a cycloalkyl group having 3-10 ring-forming carbon atoms, a heterocycloalkyl group having 3-10 ring-forming carbon atoms, an aryl group having 6-12 carbon atoms, an aryloxy group having 6-12 carbon atoms, an arylamine group having 6-12 carbon atoms, an arylphosphine group having 6-12 carbon atoms, or a heteroaryl group having 3-20 carbon atoms or 5-20 carbon atoms.

In some further embodiments of the present disclosure, each of X₁, X₂ and X₃ are N.

In some embodiments of the present disclosure, the heteroatoms in the heteroalkyl group, the heterocycloalkyl group, the heteroaryl group or the heteroarylene group are independently selected from at least one of O, S, and N.

In some embodiments of the present disclosure, the number of the heteroatoms is 1, 2, 3, 4 or 5, and/or the like. For example, the heteroatoms contain 2 N atoms and 3 N atoms and so on or may also contain 1 N atom and 1 O atom, or 1 N atom and 1 S atom, further or 2 N atoms and 2 S atoms, etc. The heteroatoms may contain multiple identical atoms at the same time or may also contain multiple different heteroatoms at the same time, etc.

In some embodiments of the present disclosure, the arylphosphine group includes at least one of monoarylphosphine group, diarylphosphine group, and triarylphosphine group.

In some embodiments of the present disclosure, the aliphatic hydrocarbyl group includes at least one of alkyl, alkenyl, and alkynyl.

In some embodiments of the present disclosure, the halogen is selected from at least one of fluorine, chlorine, bromine or iodine.

In some embodiments of the present disclosure, the halogen is selected from at least one of fluorine, chlorine or bromine.

In some embodiments of the present disclosure, the alkyl-substituted amine group having 1 to 6 carbon atoms includes a methylamine group, an ethylamine group, and a propylamine group.

In some embodiments of the present disclosure, a value of e satisfies the following conditions: 3≤e≤40. The ring A is an aliphatic ring having 3-40 ring-forming carbon atoms.

In some embodiments of the present disclosure, the aliphatic ring having 3-40 ring-forming carbon atoms is a cycloalkyl group having 3-40 ring-forming carbon atoms.

In some embodiments of the present disclosure, the ring A is an aliphatic ring having 3-20 ring-forming carbon atoms, such as an aliphatic ring having 4, 5, 6, 7, 8, 9, 10, 11, 12, 13, 14, 15, 16, 17, 18 or 19 ring-forming carbon atoms.

In some embodiments of the present disclosure, the ring A is selected from at least one of the following structural formulas:

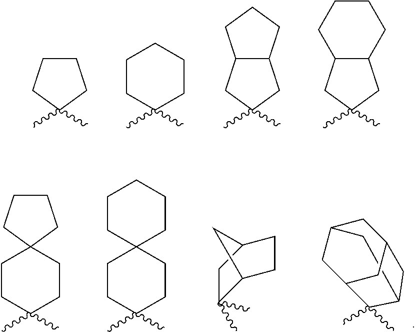

In some embodiments of the present disclosure, if the ring A contains multiple rings, R₃ and R₄ may be connected in any ring, either in the same ring or in different rings; in some further embodiments of the present disclosure, R₃ and R₄ are simultaneously connected to the ring directly connected to the fluorenyl group.

In some embodiments of the present disclosure, if the ring A contains multiple rings, c and d are independently integers≤20.

In some embodiments of the present disclosure, R₁ is deuterium and a ≥1, or R₂ is deuterium and b≥1.

In some embodiments of the present disclosure, a and b are independently selected from 2 or 3.

In some embodiments of the present disclosure, c and d are integers≤8, for example, independently selected from 1, 2, 3, 4, 5, 6 or 7, etc.

In some embodiments of the present disclosure, L is a substituted or unsubstituted arylene group, or a substituted or unsubstituted heteroarylene group.

In some embodiments of the present disclosure, L contains at least one deuterium.

In some embodiments of the present disclosure, L is selected from a substituted or unsubstituted arylene group having 6 to 60 carbon atoms, or a substituted or unsubstituted heteroarylene group having 6 to 60 carbon atoms.

In some embodiments of the present disclosure, L is selected from at least one of the following structural formulas:

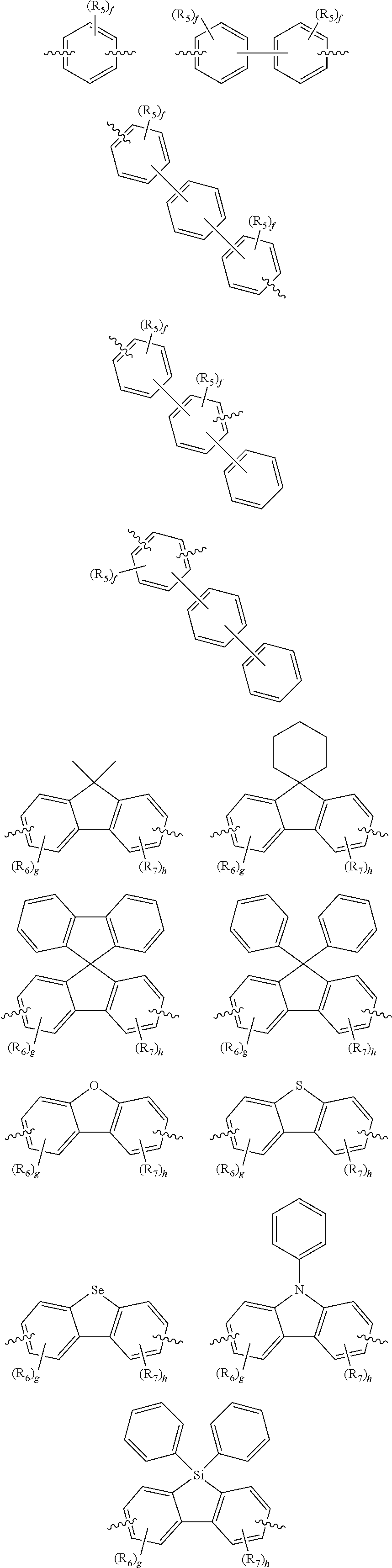

R₅, R₆ and R₇ are independently selected from deuterium, a substituted or unsubstituted alkyl group having 1 to 20 carbon atoms, a substituted or unsubstituted cycloalkyl group having 3 to 20 carbon atoms, a substituted or unsubstituted heteroalkyl group having 1-20 carbon atoms, a substituted or unsubstituted heterocycloalkyl group having 3-20 carbon atoms, a substituted or unsubstituted aryl group having 6-20 carbon atoms, or a substituted or unsubstituted heteroaryl group having 3-20 carbon atoms; and f, g and h are independently selected from an integer from 0 to 5.

In some embodiments of the present disclosure, f, g and h are independently selected from 0, 1, 2, 3, 4 or 5.

In some embodiments of the present disclosure, Ar₁ and Ar₂ are independently selected from a substituted or unsubstituted aryl group having 6-30 carbon atoms, or a substituted or unsubstituted heteroaryl group having 2-30 carbon atoms.

In some embodiments of the present disclosure, Ar₁ and Ar₂ are each independently selected from biphenyl group, naphthyl, anthracenyl, dibenzofuranyl, dibenzothienyl, 9,9-dimethylfluorenyl, 9,9-diphenylfluorenyl, aspirobifluorenyl, phenanthrenyl, pyrenyl, chrysenyl group, carbazolyl, pyridyl, pyrimidinyl, benzophenanthyl, or a substituted or unsubstituted phenyl group, or a combination of at least two of the above groups.

In some embodiments of the present disclosure, the Ar₁ is different from the Ar₂. Both of them may be the same or different. If they are the same, they can be selected from any one of the above structures; and if they are different, they can be selected from any two of the above structures.

In some embodiments of the present disclosure, the compound is selected from a group consisting of compounds having the following structural formulae:

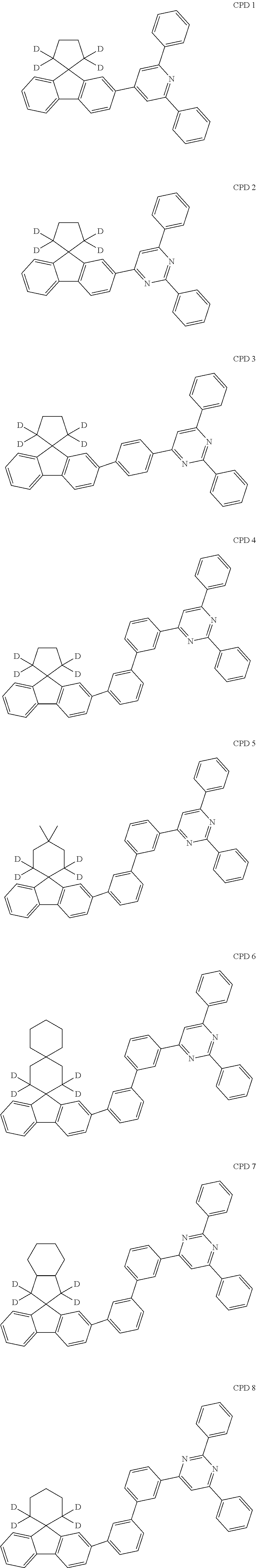

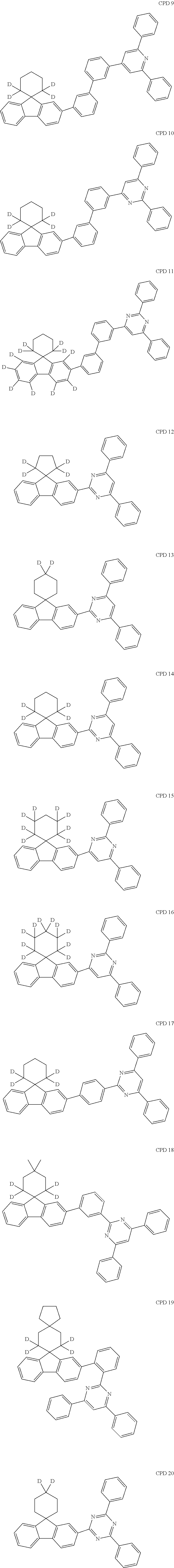

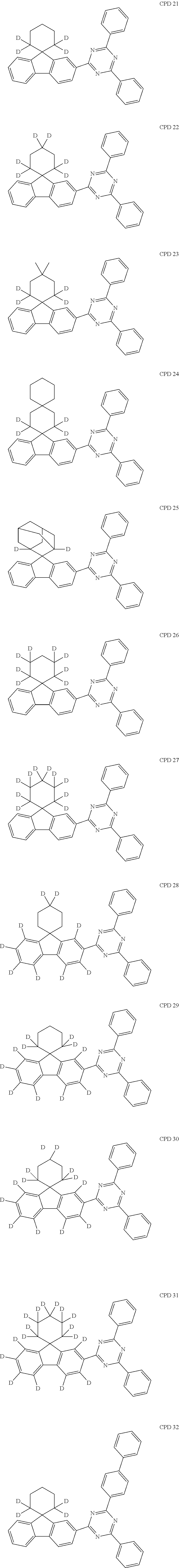

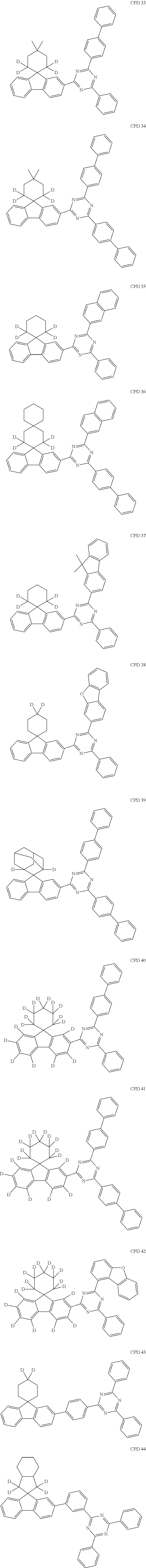

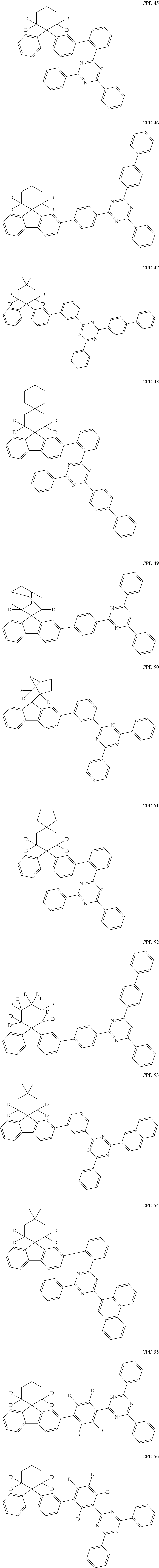

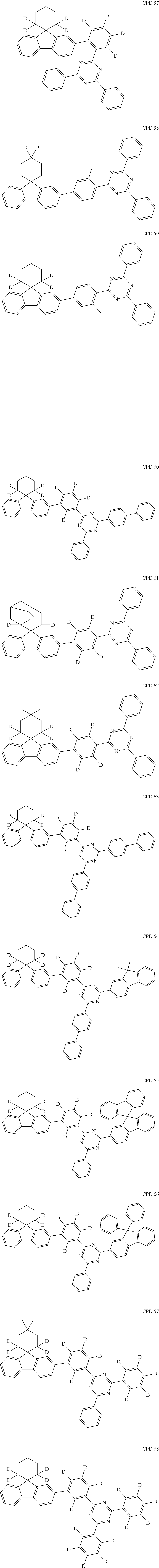

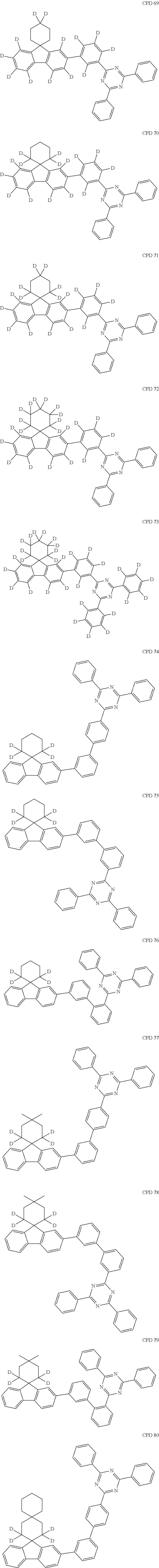

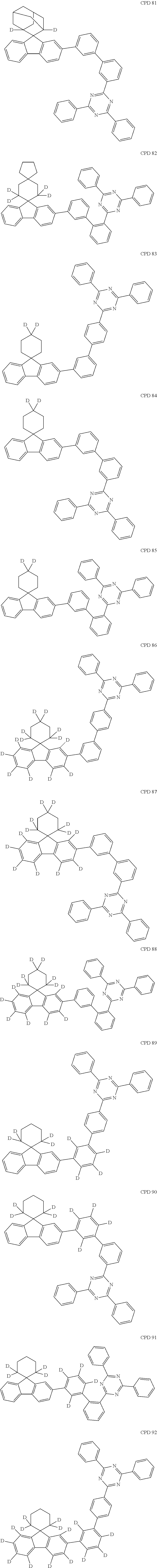

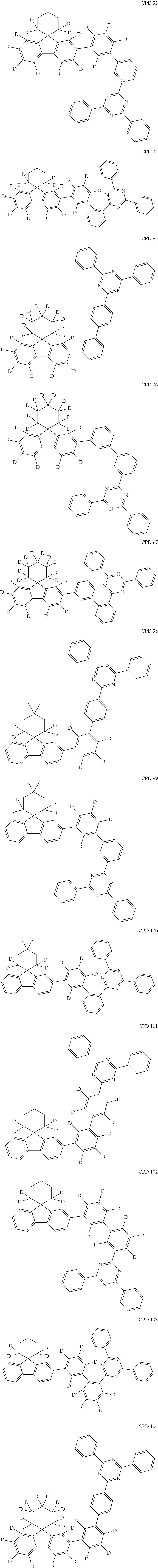

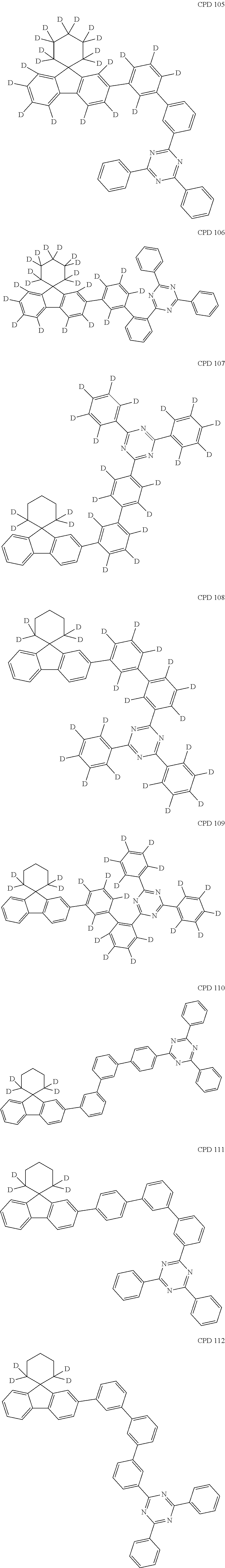

[chemical structure]

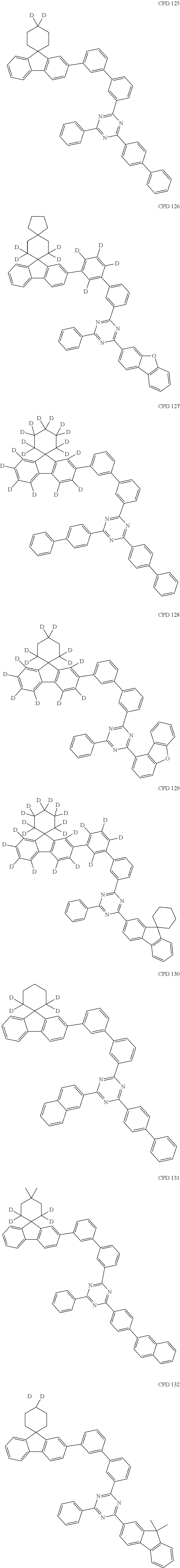

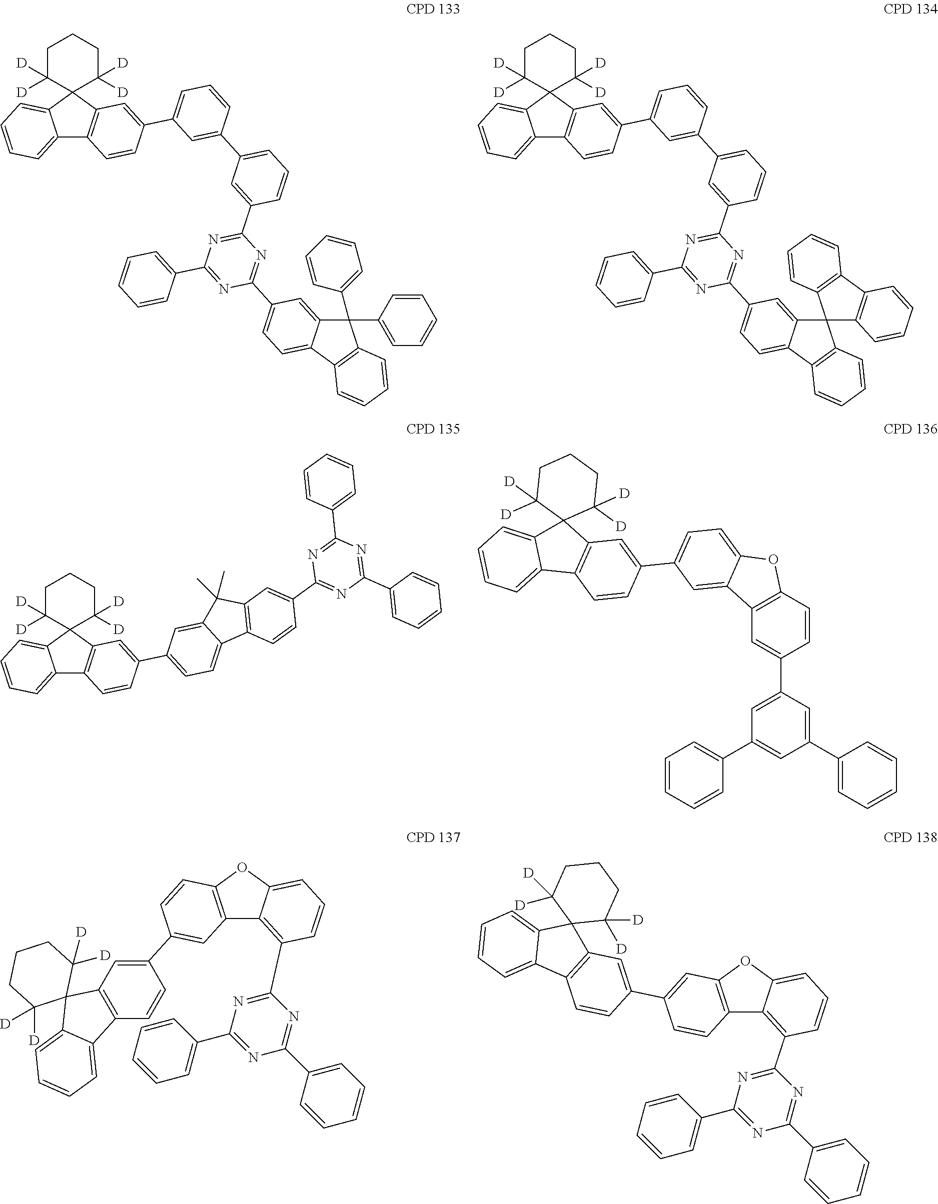

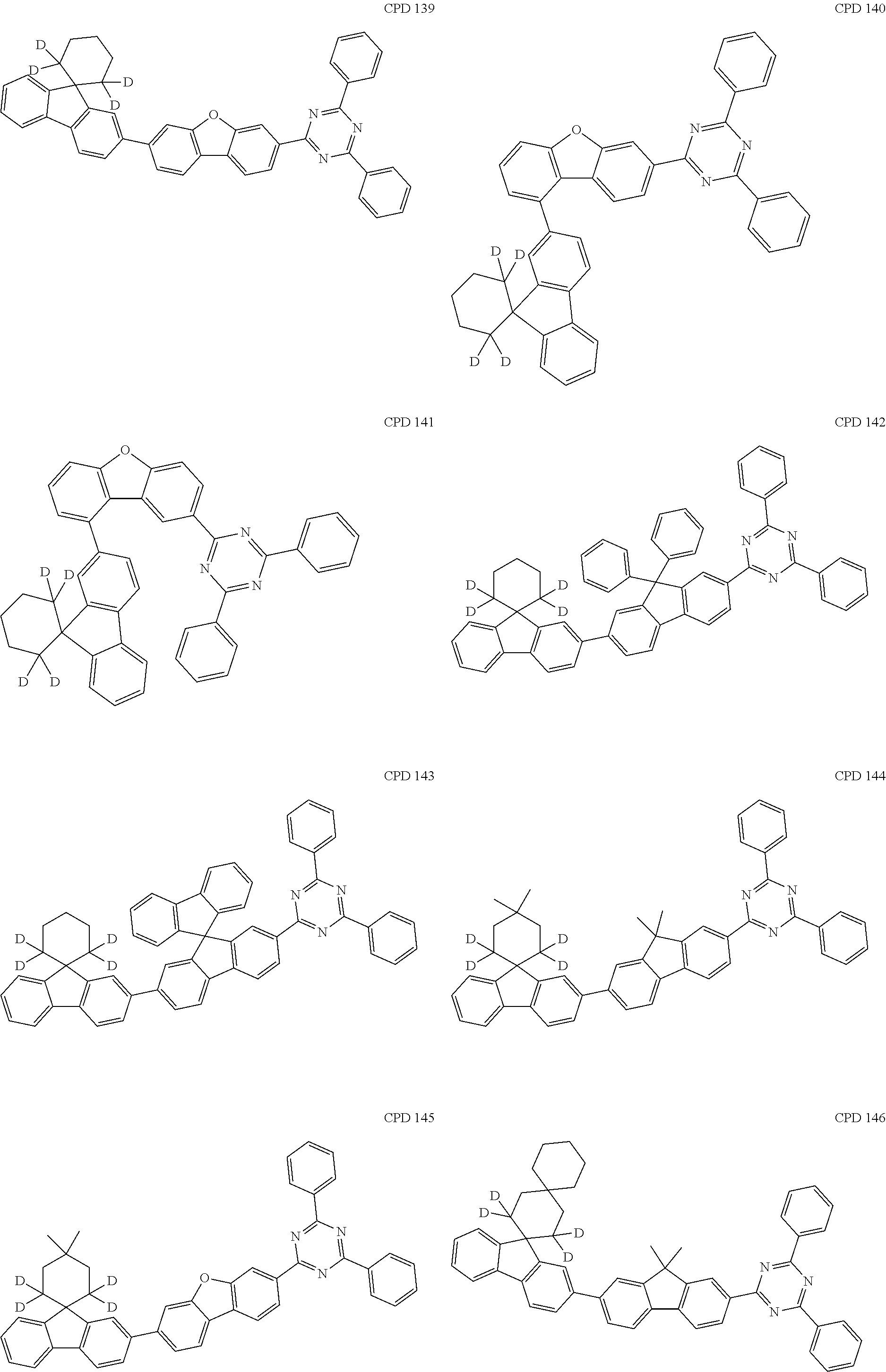

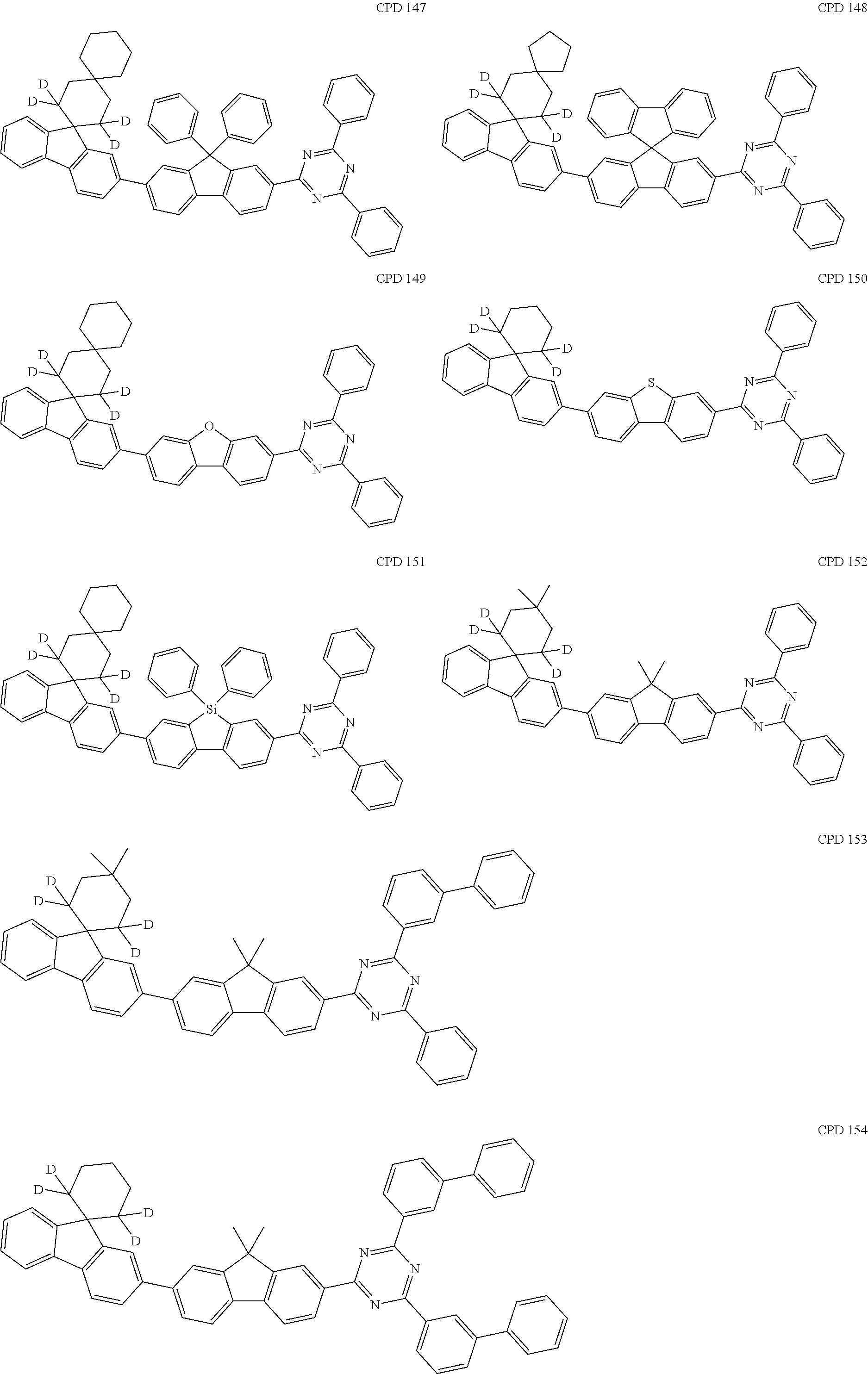

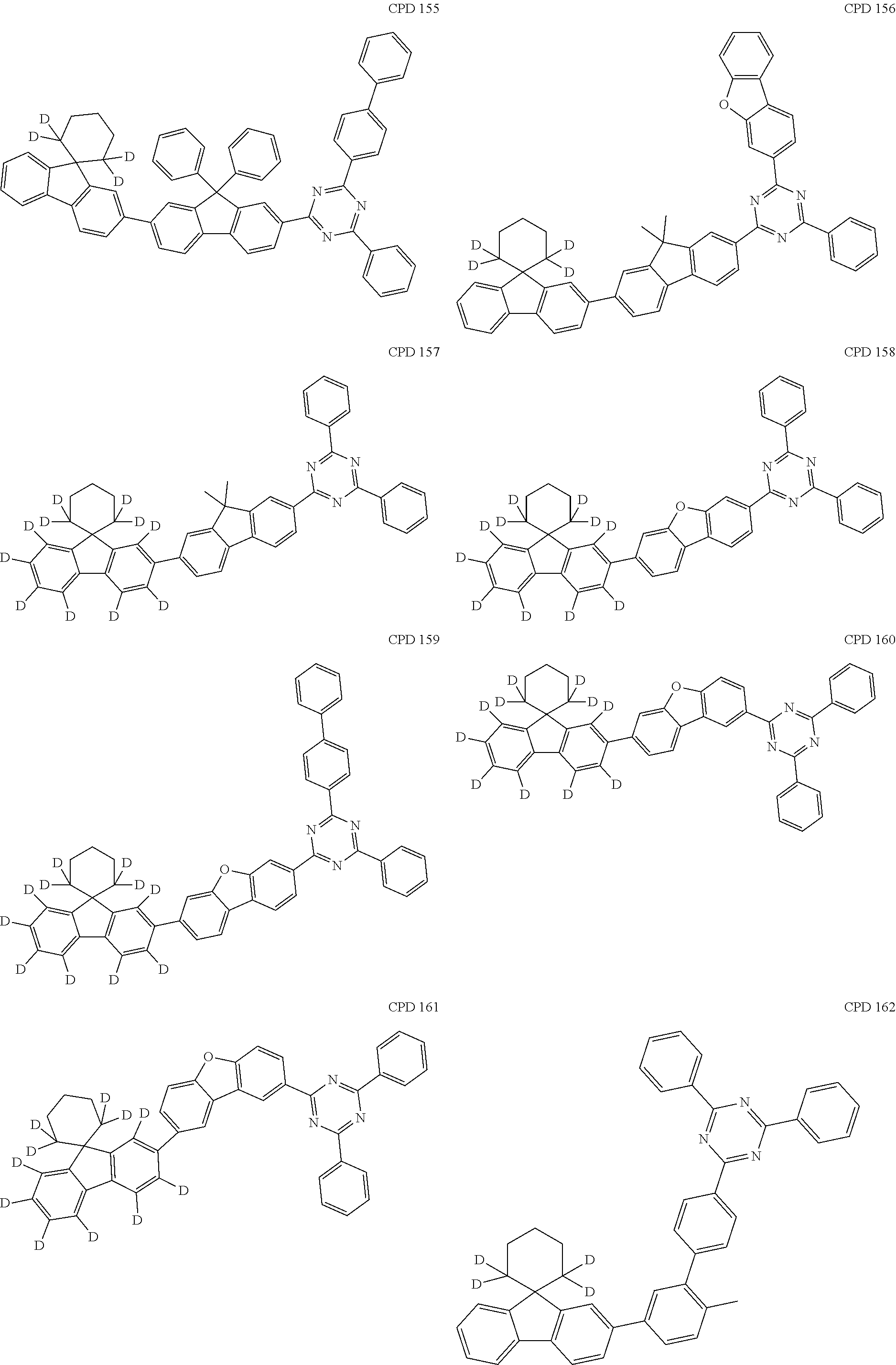

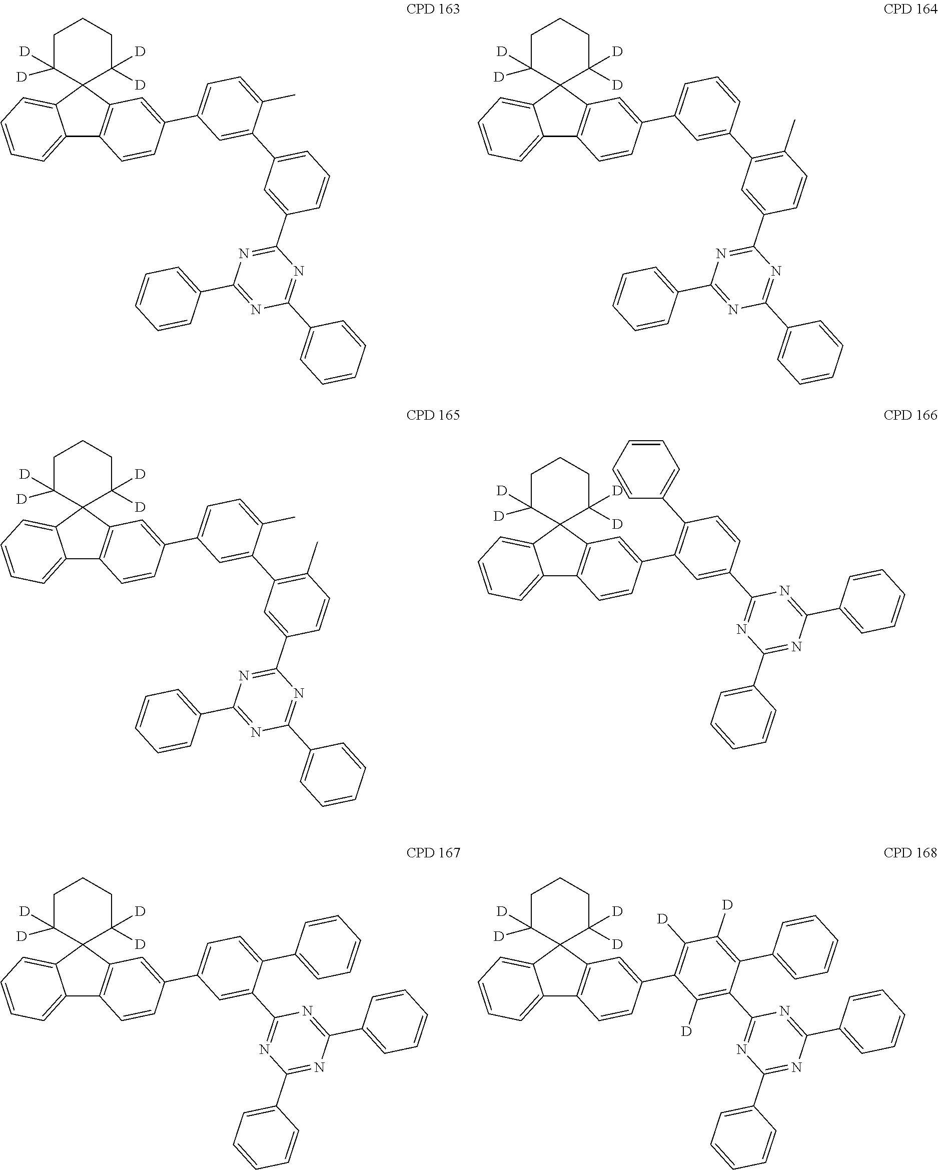

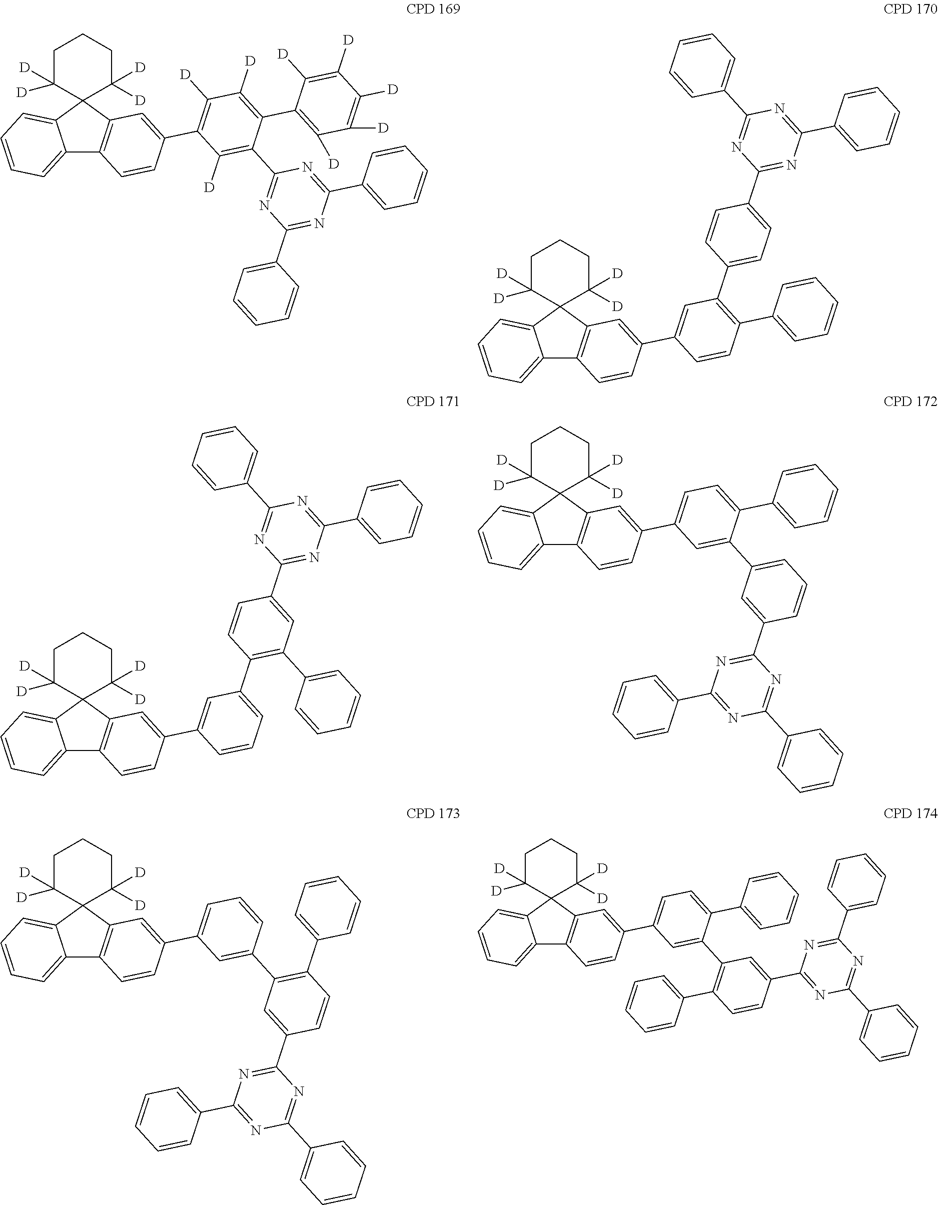

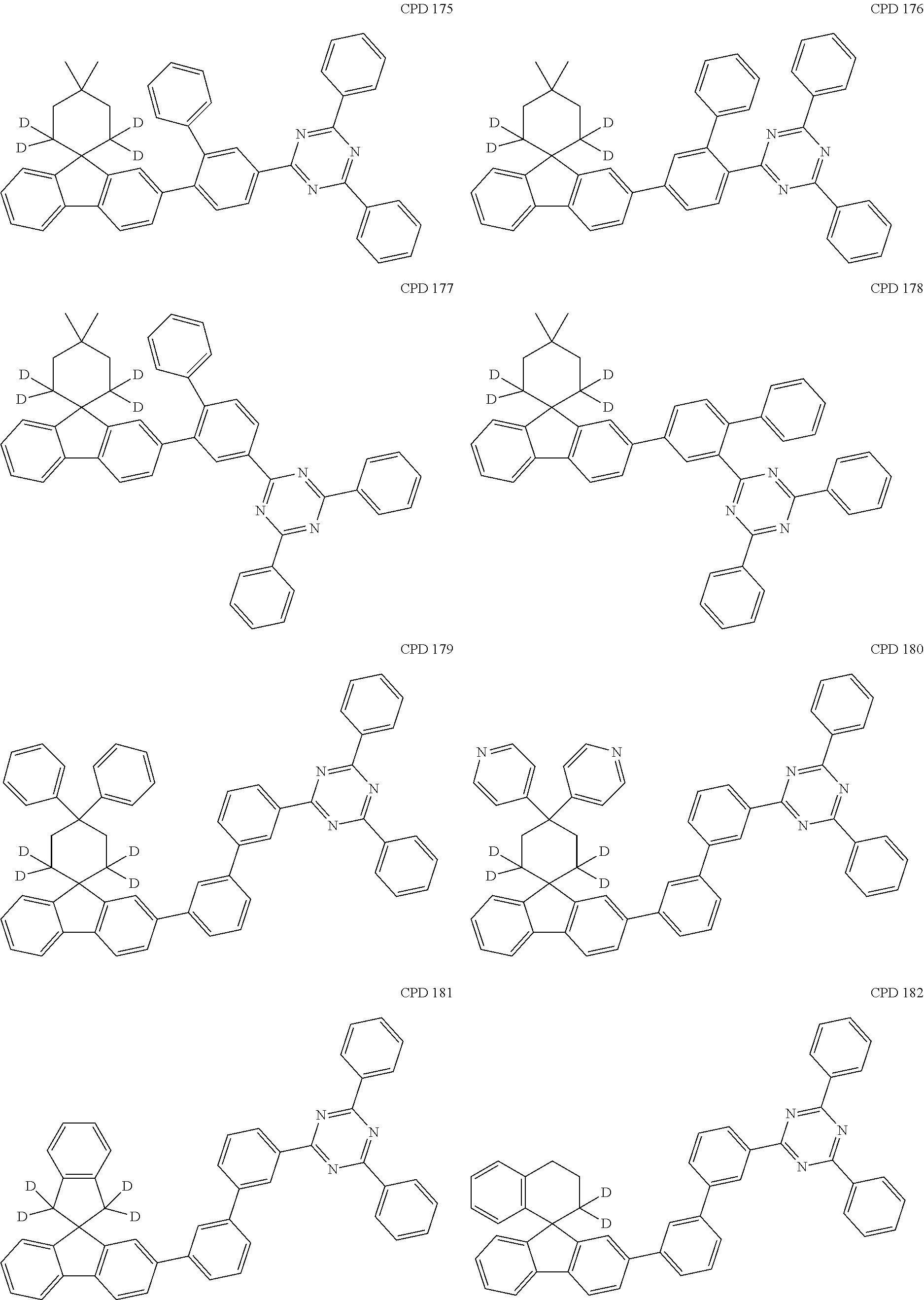

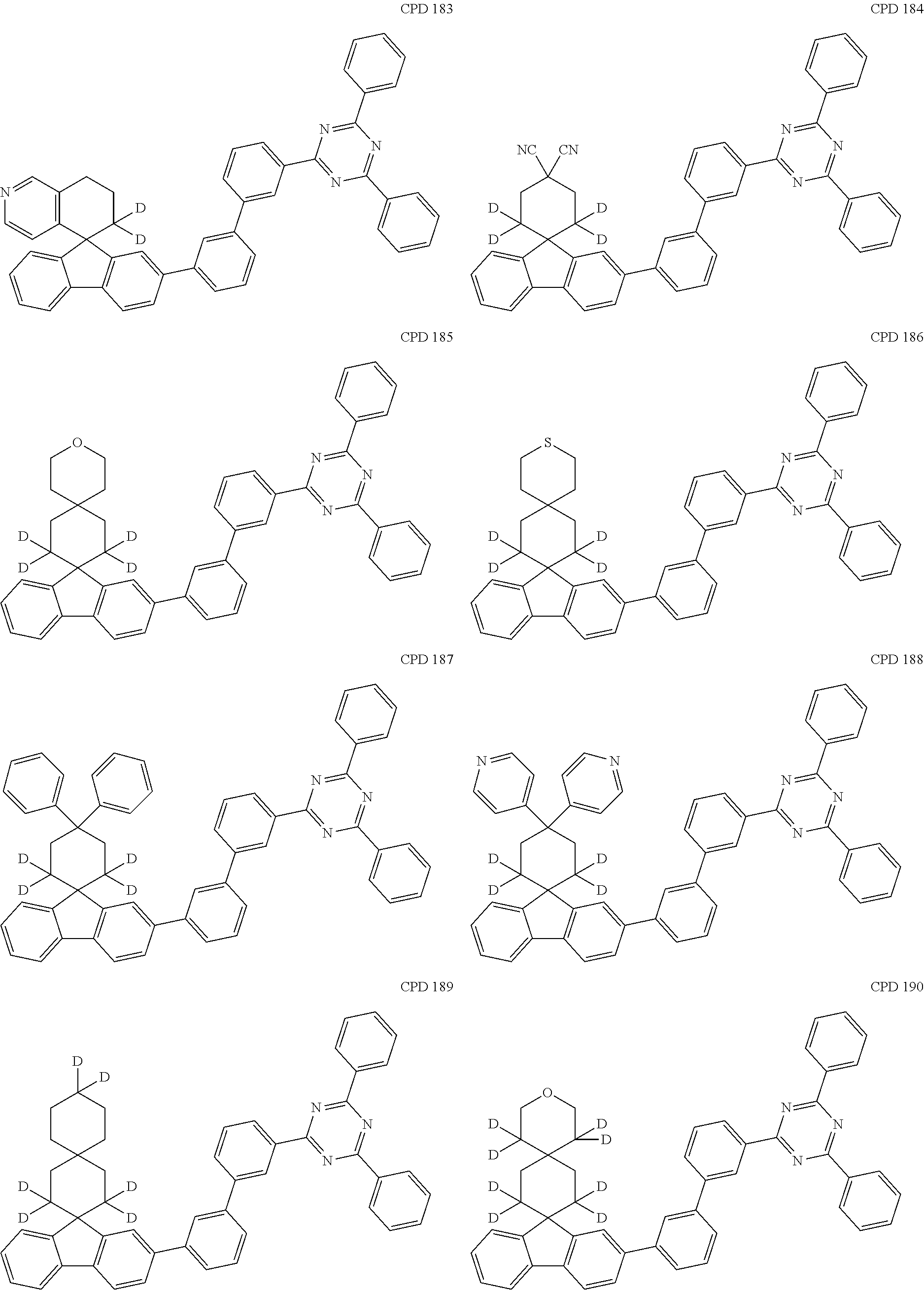

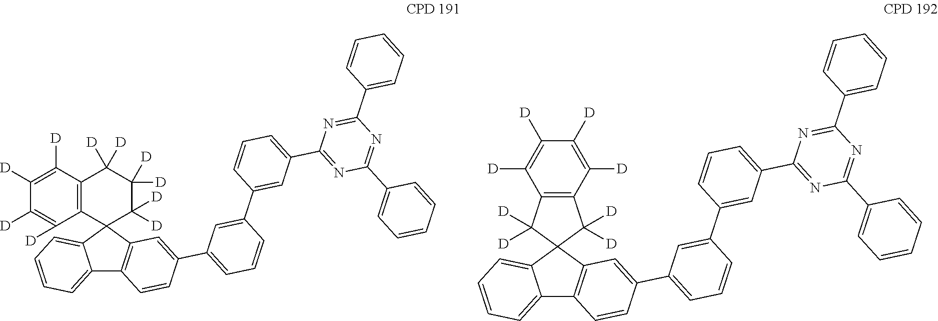 [SMILES: [2H]c1c([2H])c([2H])c2c(c1[2H])C([2H])([2H])C([2H])([2H])C([2H])([2H])C21c2ccccc2-c2ccc(-c3cccc(-c4cccc(-c5nc(-c6ccccc6)nc(-c6ccccc6)n5)c4)c3)cc21 ; [2H]c1c([2H])c([2H])c2c(c1[2H])C([2H])([2H])C1(c3ccccc3-c3ccc(-c4cccc(-c5cccc(-c6nc(-c7ccccc7)nc(-c7ccccc7)n6)c5)c4)cc31)C2([2H])[2H]]

A preparation method of the compound is further proposed herein, which includes the following steps:
S1, preparing

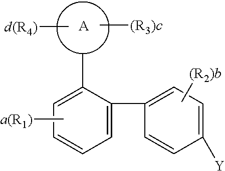

from a raw material

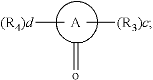

S2, preparing

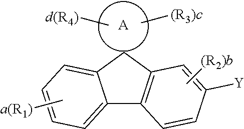

from a raw material

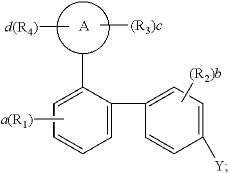

S3, preparing

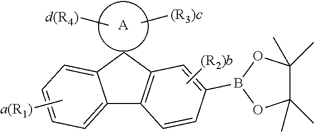

from a raw material

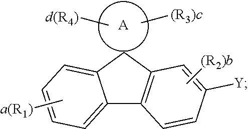

and
S4, reacting

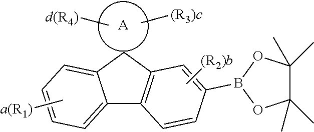

with

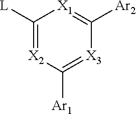

to obtain the compound, where Y represents halogen.

Use of the compound is further proposed herein. In some embodiments of the present disclosure, a functional material includes the compound, and the functional material is an organic light-emitting material, a hole blocking material or an electron transport material. The material according to the present disclosure has various potential application values in the preparation of an organic light-emitting layer, a hole electrical blocking layer or an electron transport layer. The compound according to the present disclosure may be used alone or doped to form a functional material and then be used to prepare an organic light-emitting layer, a hole blocking layer or an electron transport layer.

Alternatively, an electronic element includes a cathode and an anode. The cathode and the anode are arranged opposite to each other, an intermediate layer is provided between the cathode and the anode, and at least one layer of the intermediate layers contains the compound.

The electronic element may include an intermediate layer prepared from only one of the compounds, or may simultaneously include several intermediate layers containing the above-mentioned compounds. The intermediate layers usually include a hole injection layer, a hole transport layer, a hole injection—hole transport functional layer, a hole blocking layer, an organic light-emitting layer, an electron transport layer, an electron blocking layer, an electron injection layer, and an electron transport—electron injection functional layer, wherein the multiple material layers may be made of the above compound, or only one of the material layers may be made of the above compound. The material layer prepared from the above compound may be used as any one of the organic light-emitting layer, the hole blocking layer, and the electron transport layer.

The electronic element specifically includes at least one of an organic light-emitting diode (which may be AMOLED), an organic light-emitting battery, an organic light-emitting field-effect transistor, an organic OLED display screen, an organic light-emitting transistor (OLET), an organic light-emitting laser (OLEL), an organic light-emitting sensor (OLES), an organic light-emitting thin film transistor (OLETFT), and the like.

Further alternatively, an electronic device includes the above-mentioned electronic element.

In some embodiments of the present disclosure, the electronic device is an organic light-emitting device. The organic light-emitting device includes, but is not limited to, flat panel displays, computer monitors, medical monitors, televisions, billboards, lamps for internal or external lighting and/or signaling, head-up displays, fully or partially transparent displays, flexible displays, laser printers, telephones, mobile phones, electronic cameras notebooks, personal digital assistants (PDAs), wearable devices, laptop computers, digital cameras, camcorders, viewfinders, microdisplays, three-dimensional displays, virtual reality or augmented reality displays, vehicles, video walls with multiple displays tiled together, theater or venue screens, phototherapy devices and signs.

Further alternatively, use of the above compounds in the preparation of the organic light-emitting device is provided.

Compared with the existing technologies, the beneficial effects of the present disclosure are as follows:

The compound according to the present disclosure is formed by connecting a deuterated alkyl fluorene and a nitrogen heterocycle. This type of compounds has many advantages such as good film-forming properties, good optical, electrical and thermal stability, high luminous efficiency, low driving voltage, long lifetime and so on, and may be used in the organic light-emitting device, especially as a hole blocking material layer or an electron transport material layer, and has potential application prospects in many industries such as active matrix organic light-emitting diodes (AMOLEDs).

According to the present disclosure, “ [symbol] ” represents a connection site.

### BRIEF DESCRIPTION OF THE DRAWINGS

FIG. 1 is a schematic structural diagram of an organic light-emitting device prepared in an application example of the present disclosure; and

FIG. 2 is a nuclear magnetic resonance hydrogen spectrum of a compound CPD 8-2 prepared in Example 1 of the present disclosure.

Explanation of reference numerals: glass substrate 1; anode 2; hole injection layer 3; first hole transport layer (HTL1) 4; second hole transport layer (HTL2) 5; light-emitting layer 6; hole blocking layer (HBL) 7; electron transport layer (ETL) 8; cathode 9.

### DETAILED DESCRIPTION

In order to allow those skilled in the art to understand the technical solution of the present disclosure more clearly, the following examples are listed for description. It should be pointed out that the following examples do not limit the scope of protection claimed by the present disclosure.

Unless otherwise specified, raw materials, solvents or devices used in the following examples can be purchased from Alfa, Acros and other suppliers well known to those skilled in the art, or can be obtained by existing known methods.

A compound has the following structural formula:

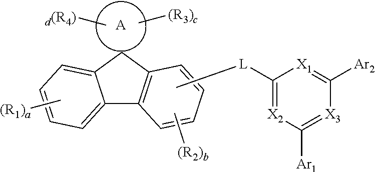

where ring A is an aliphatic ring, and the number of ring carbon atoms in the aliphatic ring is e;
X₁, X₂ and X₃ are independently selected from N or CR₀, and at least one of them is N;
R₀ is selected from hydrogen, deuterium, halogen, cyano, nitro, a substituted or unsubstituted aliphatic hydrocarbyl group with a main chain having ≤40 carbon atoms, a substituted or unsubstituted heteroalkyl group with a main chain having 1-40 carbon atoms, a substituted or unsubstituted alkoxy group with a main chain having 1-40 carbon atoms, a substituted or unsubstituted alkylsilyl group with a main chain having 1-40 carbon atoms, a substituted or unsubstituted alkylboryl group with a main chain having 1-40 carbon atoms, a substituted or unsubstituted alicyclic hydrocarbyl group having 3-40 ring-forming carbon atoms, a substituted or unsubstituted heterocycloalkyl group having 3-40 ring-forming carbon atoms, a substituted or unsubstituted aryl group having 6-60 aromatic ring carbon atoms, a substituted or unsubstituted aryloxy group having 6-60 aromatic ring carbon atoms, a substituted or unsubstituted arylsilyl group having 6-60 aromatic ring carbon atoms, a substituted or unsubstituted arylboryl group having 6-60 aromatic ring carbon atoms, a substituted or unsubstituted arylamine group having 6-60 aromatic ring carbon atoms, a substituted or unsubstituted arylphosphine group having 6-60 aromatic ring carbon atoms, or a substituted or unsubstituted heteroaryl group having 3-60 aromatic ring carbon atoms;
R₁, R₂, and R₃ are each independently selected from deuterium, halogen, cyano, nitro, a substituted or unsubstituted aliphatic hydrocarbyl group with a main chain having ≤40 carbon atoms, a substituted or unsubstituted heteroalkyl group with a main chain having 1-40 carbon atoms, a substituted or unsubstituted alkoxy group with a main chain having 1-40 carbon atoms, a substituted or unsubstituted alkylsilyl group with a main chain having 1-40 carbon atoms, a substituted or unsubstituted alkylboryl group with a main chain having 1-40 carbon atoms, a substituted or unsubstituted alicyclic hydrocarbyl group having 3-40 ring-forming carbon atoms, a substituted or unsubstituted heterocycloalkyl group having 3-40 ring-forming carbon atoms, a substituted or unsubstituted aryl group having 6-60 aromatic ring carbon atoms, a substituted or unsubstituted aryloxy group having 6-60 aromatic ring carbon atoms, a substituted or unsubstituted arylsilyl group having 6-60 aromatic ring carbon atoms, a substituted or unsubstituted arylboryl group having 6-60 aromatic ring carbon atoms, a substituted or unsubstituted arylamine group having 6-60 aromatic ring carbon atoms, a substituted or unsubstituted arylphosphine group having 6-60 aromatic ring carbon atoms, or a substituted or unsubstituted heteroaryl group having 3-60 aromatic ring carbon atoms;
R₄ is selected from deuterium or R₈ group substituted by deuterium;
R₈ is selected from a substituted or unsubstituted aliphatic hydrocarbyl group with a main chain having ≤40 carbon atoms, a substituted or unsubstituted heteroalkyl group with a main chain having 1-40 carbon atoms, a substituted or unsubstituted alkoxy group with a main chain having 1-40 carbon atoms, a substituted or unsubstituted alkylsilyl group with a main chain having 1-40 carbon atoms, a substituted or unsubstituted alkylboryl group with a main chain having 1-40 carbon atoms, a substituted or unsubstituted alicyclic hydrocarbyl group having 3-40 ring-forming carbon atoms, a substituted or unsubstituted heterocycloalkyl group having 3-40 ring-forming carbon atoms, a substituted or unsubstituted aryl group having 6-60 aromatic ring carbon atoms, a substituted or unsubstituted aryloxy group having 6-60 aromatic ring carbon atoms, a substituted or unsubstituted arylsilyl group having 6-60 aromatic ring carbon atoms, a substituted or unsubstituted arylboryl group having 6-60 aromatic ring carbon atoms, a substituted or unsubstituted arylamine group having 6-60 aromatic ring carbon atoms, a substituted or unsubstituted arylphosphine group having 6-60 aromatic ring carbon atoms, or a substituted or unsubstituted heteroaryl group having 3-60 aromatic ring carbon atoms;
a is an integer selected from 0-4; if a is an integer≥2, the adjacent R₁ are connected to each other to form a fused ring or not connected; b is an integer selected from 0 to 3; if b is an integer≥2, the adjacent R₂ are connected to each other to form a fused ring or not connected; c is selected from an integer≥0; d is selected from an integer≥1; and e is selected from an integer≥3;
L is selected from a single bond, a substituted or unsubstituted arylene group, or a substituted or unsubstituted heteroarylene group;
Ar₁ and Ar₂ are independently selected from a substituted or unsubstituted aryl group, or a substituted or unsubstituted heteroaryl group;
heteroatoms in the heteroalkyl group, the heterocycloalkyl group, the heteroaryl group or the heteroarylene group are independently selected from at least one of O, S, N, Se, Si and Ge; and
the term “substituted” refers to being substituted by at least one of deuterium, halogen, cyano, isocyano, phosphine group, an alkyl group having 1-6 carbon atoms, a cycloalkyl group having 3-16 ring carbon atoms, an alkyl-substituted amine group having 1-6 carbon atoms, an aryl group having 6-12 carbon atoms, or a deuterated aryl group having 6-12 carbon atoms, where the substituted number ranges from a monosubstitution to a maximum substitution. The adjacent R₁ refer to R₁ located on the same carbon atom or adjacent carbon atoms, and the adjacent R₂ refer to R₂ located on the same carbon atom or adjacent carbon atoms.

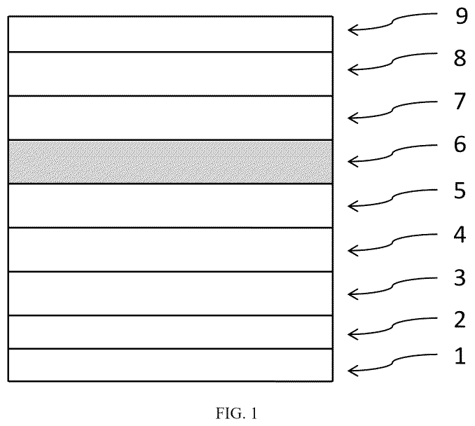

The above compounds can be applied to organic light-emitting devices. In some embodiments of the present disclosure, as shown in FIG. 1, the organic light-emitting device includes stacked glass substrate 1, anode 2, hole injection layer 3, first hole transport layer (HTL1) 4, second hole transport layer (HTL2) 5, light-emitting layer 6, hole blocking layer (HBL) 7, electron transport layer (ETL) 8 and cathode 9.

The preparation method of the above-mentioned organic light-emitting device includes:
the glass substrate with an ITO transparent electrode (anode 2) is ultrasonically cleaned in ethanol for 10 min, dried at 150° C. and treated with N₂ Plasma for 30 minutes. The washed glass substrate is mounted on a substrate holder of a vacuum evaporation device. First, a compound HATCN is evaporated on the side of the transparent electrode line to cover the transparent electrode to form a thin film (hole injection layer 3). Then a layer of compound HTM1 is evaporated to form a thin film as HTL1 (first hole transport layer 4), and then a layer of compound HTM2 is evaporated on the compound HTM1 film to form a film as HTL2 (second hole transport layer 5), and then a host material compound and a guest material compound are evaporated in a co-evaporation mode on the HTM2 film layer to form a light-emitting layer. A hole blocking layer (HBL) and an electron transport layer are sequentially formed on the light-emitting layer using an evaporation method. The material of the hole blocking layer is the compound of the embodiment of the present disclosure or the comparative compound, and the material of the electron transport layer is the electron transport layer material ETL or the compound of the present disclosure: LiQ. Then, Mg/Ag are evaporated as the cathode material in the co-evaporation mode.

The structural formulas of the above-mentioned compounds HATCN, HTM1, HTM2, host material compounds, guest material compounds, ETL, and LiQ are as follows:

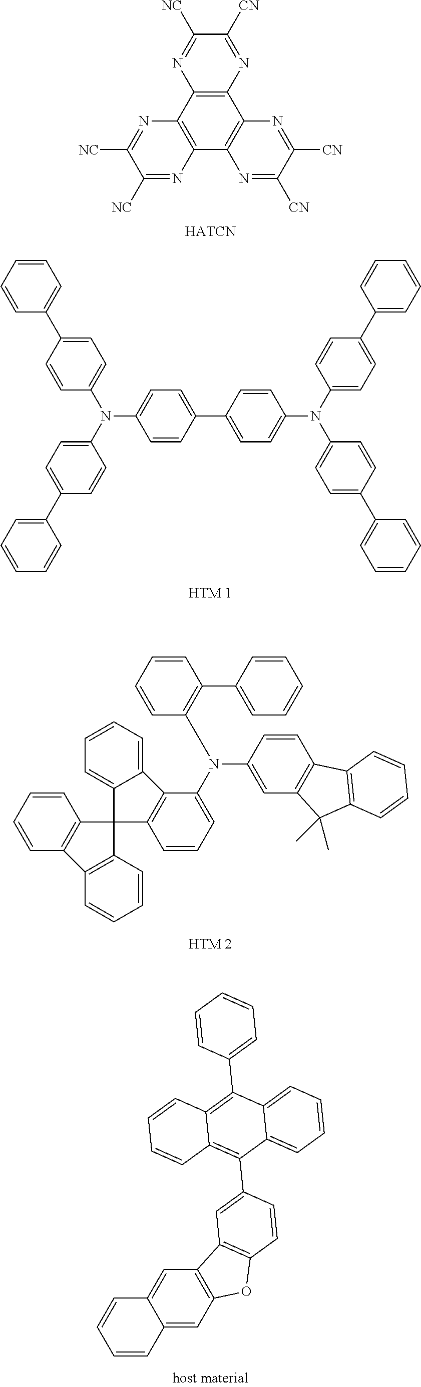

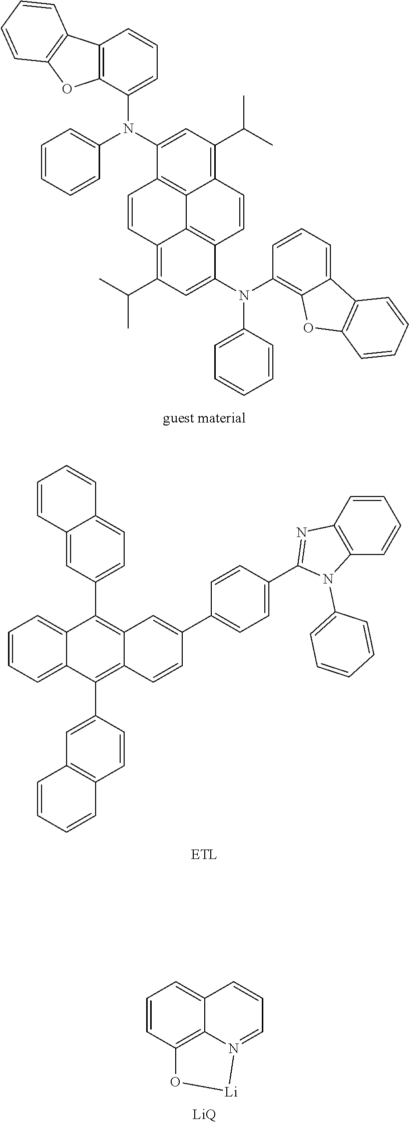

### Example 1

In this example, a compound (recorded as CPD 8) was prepared, and its preparation route was as follows:

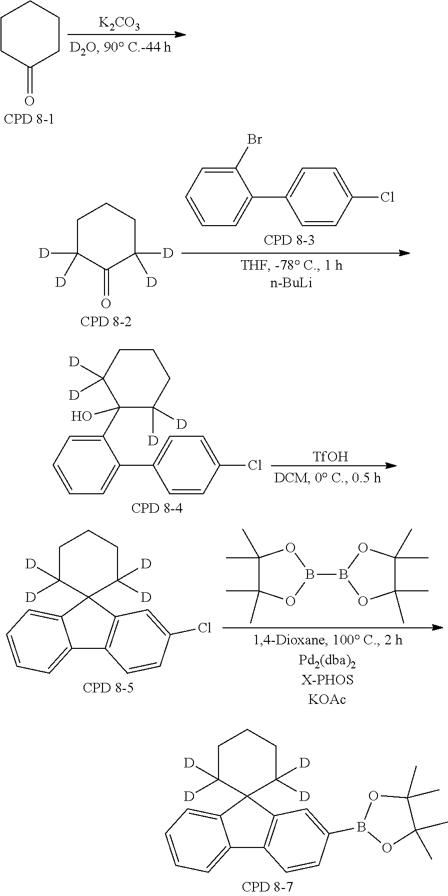

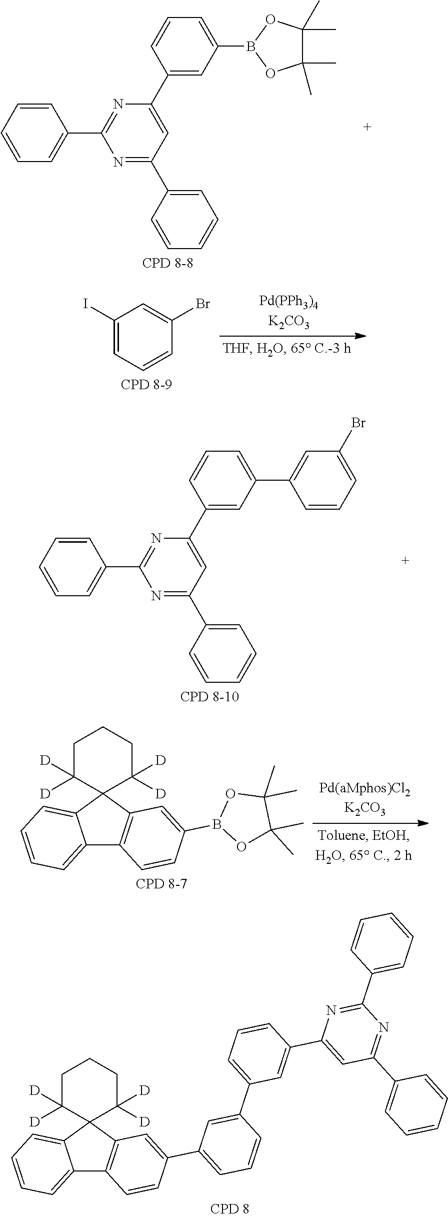

The specific preparation process was:

### 1) Synthesis of Compound CPD 8-2

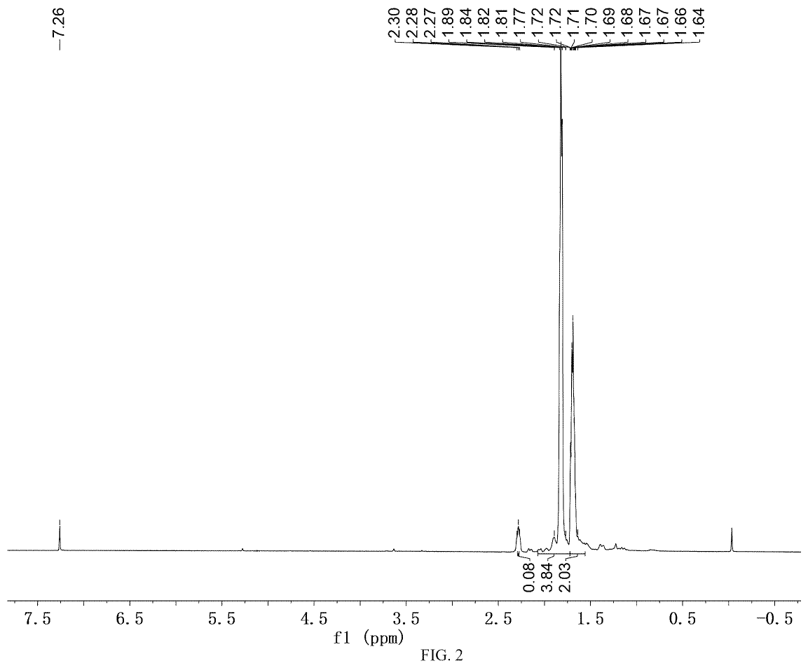

CPD 8-1 (50.00 g, 0.51 mol), anhydrous potassium carbonate (140.97 g, 1.02 mmol) and heavy water (300 g) were added into a 1,000 mL three-necked round-bottomed flask, pumping vacuum and purging with nitrogen gas for three times. A reaction system was sealed by a reverse rubber stopper and a raw material tape seal, and then the system was heated to 90° C. to react for 44 h and monitored by HR-MS and hydrogen spectroscopy. When the 4D deuteration content of the raw material CPD 8-2 was around 98%, the system was stopped heating. After the system was cooled to room temperature, liquid separation was performed directly to achieve an organic phase. Anhydrous magnesium sulfate (10 g) was added to the organic phase and stirred at room temperature for 30 min, and then diatomaceous earth (30 g) was laid for suction filtration to obtain a colorless liquid CPD 8-2 (44.50 g, purity: 99.90%). CDCl₃ was used as a deuterated reagent to measure the hydrogen spectrum, and the results were shown in FIG. 2. The integral of the remaining undeuterated hydrogen at position 2.10 of δ was 0.0812, so the deuterated content of the four D was calculated as [(4−0.0812)×102.17]/[(4−0.0812)×102.17+0.0812×98.15]=98.04%, mass spectrometry: 102.10 (GC-MS).

### 2) Synthesis of Compound CPD 8-4

Compound CPD 8-3 (50.00 g, 186.88 mmol) and dry tetrahydrofuran (750 mL) were added to a 2,000 mL three-necked round-bottomed flask, pumping vacuum and purging with nitrogen gas for three times. Then the system was cooled to −78° C., and a solution of n-butyllithium solution in n-hexane (97.18 mL, 242.95 mmol, a concentration of 2.5 mol/L) was added dropwise. The temperature of the system was controlled not to be higher than −70° C., and the solution of n-butyllithium solution in n-hexane was added dropwise over 1 h, and stirred at −78° C. for 1 h. Finally, CPD 8-2 (26.73 g, 261.63 mmol) was slowly added dropwise over 10 min, and further stirred at −78° C. for 1 h. TLC (ethyl acetate:n-hexane=1:30 as a developing solvent) monitored that the raw material CPD 8-3 was consumed completely and most of the CPD 8-4 was generated. Deionized water was added dropwise to the system to quench the reaction (500 mL). After rising to room temperature, the liquids were separated directly, where an aqueous phase was extracted twice with ethyl acetate (400 mL×2), the combined organic phase was concentrated under reduced pressure at 65° C. for 1 h to obtain a light yellow liquid. The liquid was mixed with silica gel and put on the column by dry method for purification by silica gel column chromatography (200-300 mesh silica gel, ethyl acetate:n-hexane=1:30 as the eluent). After elution, the purified solution was concentrated under reduced pressure at 75° C. for 1 h to obtain a colorless liquid as CPD 8-4 (44.63 g, purity: 99.31%, yield: 82.11%), mass spectrum: 291.14 (M+H).

### 3) Synthesis of Compound CPD 8-5

CPD 8-4 (42.00 g, 144.42 mmol) and dichloromethane (600 mL) were added to a 1,000 mL three-necked round-bottomed flask, and the system was then cooled to 0° C., and trifluoromethanesulfonic acid (65.02 g, 433.25 mmol) was added dropwise over 10 min and stirred at 0° C. for 30 min. TLC (ethyl acetate:n-hexane=1:30 as a developing solvent) monitored that the raw material CPD 8-4 was consumed completely. Deionized water was added dropwise to the system to quench the reaction (200 mL), and the liquids were separated directly and mixed with silica gel and put on the column by dry method for purification by silica gel column chromatography (200-300 mesh silica gel, n-hexane=100% as the eluent). After elution, the purified solution was concentrated under reduced pressure at 75° C. for 1 h to obtain n a white solid was CPD 8-5 (29.68 g, purity: 99.41%, yield: 75.33%), mass spectrum: 273.13 (M+H).

### 4) Synthesis of Compound CPD 8-7

CPD 8-5 (28.00 g, 102.64 mmol), CPD 8-6 (31.28 g, 123.16 mmol), tris(dibenzylideneacetone)dipalladium (1.87 g, 2.05 mmol), 2-dicyclohexylphosphine-2′,4′,6′-triisopropyl biphenyl (1.95 g, 4.10 mmol), potassium acetate (20.14 g, 205.28 mmol) and 1,4-dioxane (420 mL) were added to a 1,000 mL three-necked round-bottomed flask, pumping vacuum and purging with nitrogen gas for three times. Then, the system was heated to 100° C. to react for 2 h, and TLC (ethyl acetate:n-hexane=1:10 as the developing solvent) monitored that the raw material CPD 8-5 was consumed completely. The system was cooled to 60° C., and concentrated under reduced pressure to remove the solvent, and ethyl acetate (800 mL) was added to the concentrated system. The system was washed twice in deionized water (300 mL×2), and the obtained liquids were separated directly and mixed with silica gel and put on the column by dry method for purification by silica gel column chromatography (200-300 mesh silica gel, ethyl acetate:n-hexane=1:15 as the eluent). After elution, the purified solution was concentrated under reduced pressure at 75° C. for 1 h to obtain a white solid as CPD 8-7 (28.17 g, purity: 98.75%, yield: 75.33%), mass spectrum: 365.26 (M+H).

### 5) Synthesis of Compounds CPD 8-10

CPD 8-8 (25.00 g, 57.56 mmol), CPD 8-9 (19.54 g, 69.07 mmol), tetrakis(triphenylphosphine)palladium (1.33 g, 1.15 mmol), potassium carbonate (15.91 g, 115.12 mmol), tetrahydrofuran (375 mL) and deionized water (125 mL) were added into a 1,000 mL three-necked round-bottomed flask, pumping vacuum and purging with nitrogen gas for three times. Then, the system was heated to 65° C. to react for 3 h, and TLC (ethyl acetate:n-hexane=1:7 as the developing solvent) monitored that the raw material CPD 8-8 was consumed completely. The system was concentrated under reduced pressure to remove the solvent, and ethyl acetate (600 mL) was added to the concentrated system. The system was washed twice in deionized water (300 mL×2), and the obtained liquids were separated directly and mixed with silica gel and put on the column by dry method for purification by silica gel column chromatography (200-300 mesh silica gel, ethyl acetate:n-hexane=1:10 as the eluent). After elution, the purified solution was concentrated under reduced pressure at 70° C. for 1 h to obtain a white solid as CPD 8-10 (20.98 g, purity: 99.68%, yield: 78.65%), mass spectrum: 463.08 (M+H).

### 6) Synthesis of Compound CPD 8

CPD 8-10 (16.00 g, 34.53 mmol), CPD 8-7 (14.90 g, 37.98 mmol), bis(4-dimethylaminophenyldi-tert-butylphosphine)palladium dichloride (0.49 g, 0.69 mmol), potassium carbonate (9.55 g, 69.06 mmol), toluene (240 mL), ethanol (80 mL) and deionized water (80 mL) were added to a 1,000 mL three-necked round-bottomed flask, pumping vacuum and purging with nitrogen gas for three times. Then the system was heated to 65° C. and reacted for 2 h. TLC (ethyl acetate:n-hexane=1:10 as the developing solvent) monitored that the raw materials CPD 8-10 were consumed completely. The system was cooled to room temperature, added with methanol (300 mL) and stirred at room temperature for 1 h, and a large amount of solid precipitated. Toluene (500 mL) was added, and the system was warmed to 100° C. and dissolved, then cooled to room temperature, and filtered once with 300-400 mesh silica gel (60 g). Methanol (50 mL) was added to the filtrate at room temperature, stirred at room temperature for 0.5 h, filtered with suction to obtain a white wet solid. The obtained white wet solid was dried at 80° C. for 1 h to obtain 18.54 g white solid. The dried white solid was added to a 500 mL single-necked round-bottomed flask, recrystallized twice with toluene (93 mL) and methanol (93 mL) and filtered with suction. The obtained filter cake was dried under vacuum at 80° C. for 3 h to obtain a white solid as CPD 8 (15.46 g, purity: 99.94%, yield: 72.14%). 15.46 g of the crude CPD 8 were purified by sublimation to obtain a sublimated pure CPD 8 (12.57, purity: 99.95%, yield: 81.31%), mass spectrum: 621.22 (M+H). ¹H NMR (400 MHz, CDCl₃) δ 8.16 (d, J=7.7 Hz, 1H), 8.10-8.02 (m, 2H), 7.97-7.89 (m, 1H), 7.92-7.88 (m, 2H), 7.88-7.81 (m, 2H), 7.81-7.75 (m, 2H), 7.74-7.68 (m, 2H), 7.62-7.54 (m, 3H), 7.54-7.40 (m, 10H), 7.32-7.30 (m, 1H), 2.08-2.03 (m, 2H), 1.95-1.90 (m, 2H), 1.53-1.49 (m, 2H).

### Example 2

In this example, a compound (recorded as CPD 34) was prepared, and its preparation route was as follows:

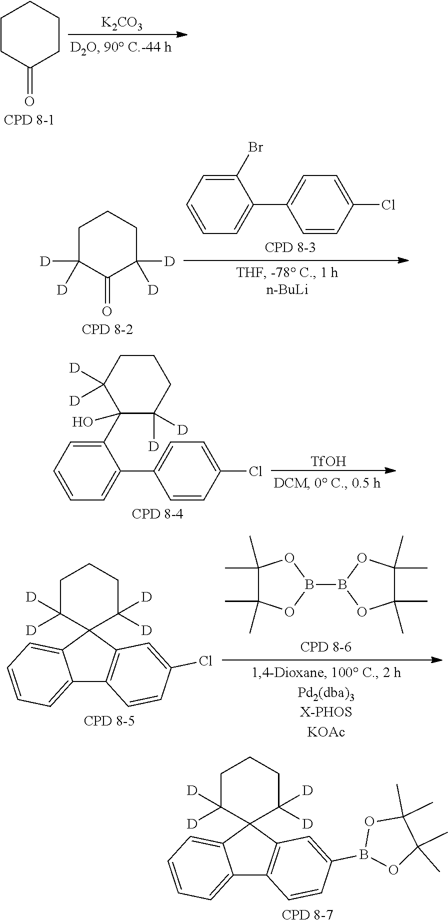

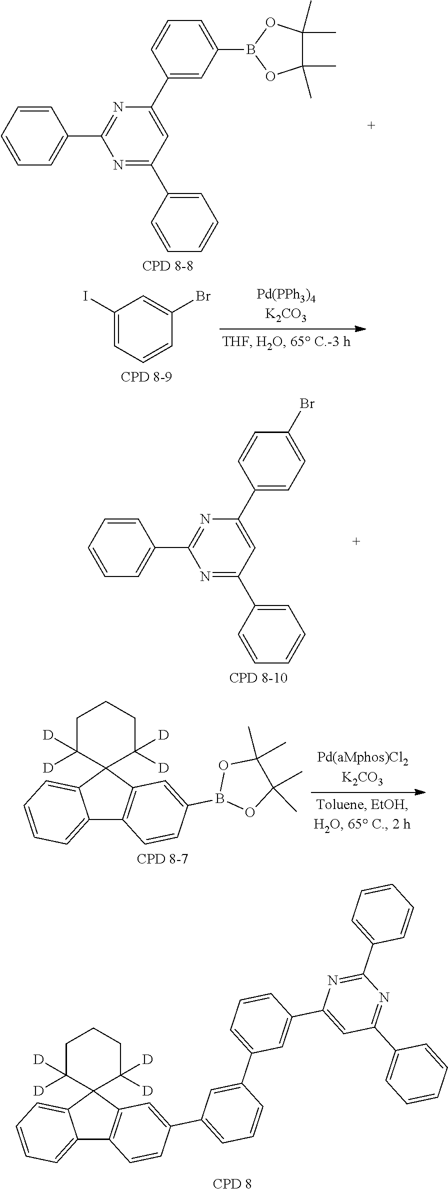

### 1) Synthesis of Compound CPD 34-2

Referring to the synthesis and purification method of compound CPD 8-2, it was only necessary to change the corresponding raw materials to obtain a target compound CPD 34-2 (30.05 g, purity: 99.68%, the deuteration content of four D was 97.88%, yield: 88.78%), mass spectrum: 130.12 (GC-MS).

### 2) Synthesis of Compound CPD 34-3

Referring to the synthesis and purification method of compound CPD 8-4, it was only necessary to change the corresponding raw materials to obtain a target compound CPD 34-3 (40.01 g, purity: 99.20%, yield: 78.65%), mass spectrum: 319.12 (M+H).

### 3) Synthesis of Compound CPD 34-4

Referring to the synthesis and purification method of compound CPD 8-5, it was only necessary to change the corresponding raw materials to obtain a target compound CPD 34-4 (29.85 g, purity: 99.52%, yield: 74.33%), mass spectrum: 301.42 (M+H).

### 4) Synthesis of Compound CPD 34-5

Referring to the synthesis and purification method of compound CPD 8-7, it was only necessary to change the corresponding raw materials to obtain a target compound CPD 34-5 (27.25 g, purity: 99.01%, yield: 76.52%), mass spectrum: 393.02 (M+H).

### 5) Synthesis of Compound CPD 34

CPD 34-6 (17.32 g, 41.25 mmol), CPD 34-5 (17.80 g, 45.37 mmol), tetrakis(triphenylphosphine)palladium (0.96 g, 0.83 mol), sodium hydroxide (3.30 g, 82.50 mmol), tetrahydrofuran (260 mL) and deionized water (90 mL) were added to a 1,000 mL three-necked round-bottomed flask, pumping vacuum and purging with nitrogen gas for three times. Then the system was heated to 75° C. to react for 3 h. TLC (ethyl acetate:n-hexane=1:10 as the developing solvent) monitored that the raw materials CPD 34-6 were consumed completely. The system was cooled to room temperature, added with methanol (300 mL) and stirred at room temperature for 1 h, and a large amount of solid precipitated. Toluene (400 mL) was added, and the system was warmed to 100° C. and dissolved, then cooled to room temperature, and filtered once with 300-400 mesh silica gel (50 g). Methanol (60 mL) was added to the filtrate at room temperature, stirred at room temperature for 0.5 h, filtered with suction to obtain a white wet solid. The obtained white wet solid was dried at 80° C. for 1 h to obtain 22.14 g white solid. The dried white solid was added to a 500 mL single-necked round-bottomed flask, recrystallized twice with toluene (220 mL) and methanol (220 mL) and filtered with suction. The obtained filter cake was dried under vacuum at 80° C. for 3 h to obtain a white solid as CPD 34 (20.00 g, purity: 99.94%, yield: 74.26%). 20.00 g of the crude CPD 34 were purified by sublimation to obtain a sublimated pure CPD 34 (16.48 g, purity: 99.96%, yield: 82.40%), mass spectrum: 650.32 (M+H). ¹HNMR (400 MHz, CDCl₃) δ 8.23 (d, J=2.1 Hz, 1H), 8.06-8.05 (m, 1H), 8.02-7.96 (m, 2H), 7.96-7.90 (m, 4H), 7.70-7.68 (m, 4H), 7.60-7.54 (m, 4H), 7.51-7.47 (m, 2H), 7.42-7.40 (m, 6H), 7.33-7.30 (m, 1H), 2.56 (s, 4H), 0.84 (s, 6H).

### Example 3

In this example, a compound (recorded as CPD 60) was prepared, and its preparation route was as follows:

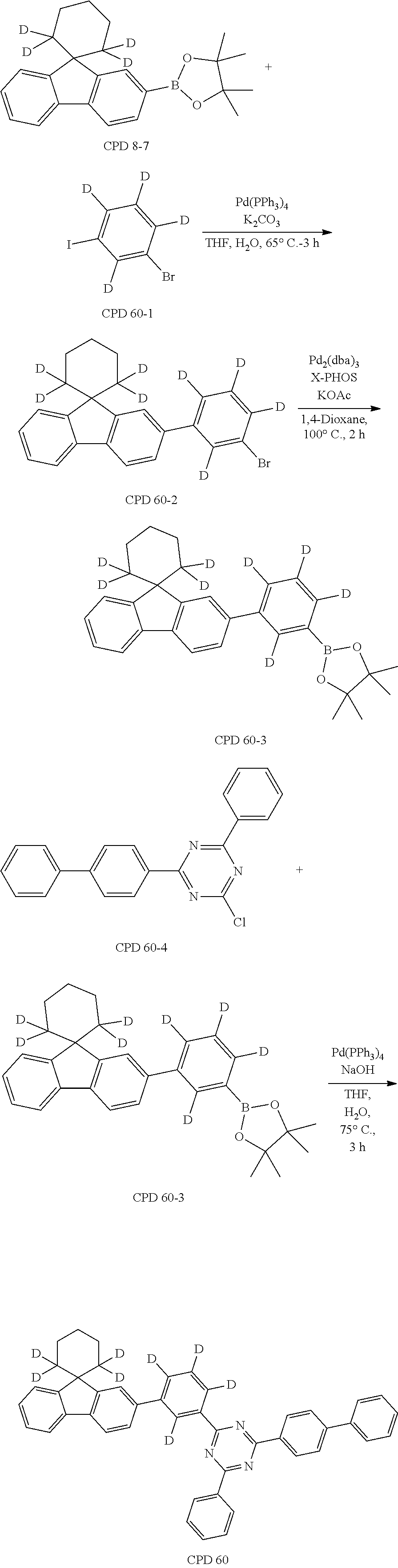

### 1) Synthesis of Compound CPD 60-2

Referring to the synthesis and purification method of compound CPD 8-10, it was only necessary to change the corresponding raw materials to obtain a target compound CPD 60-2 (33.54 g, purity: 99.56%, yield: 77.89%), mass spectrum: 397.12 (M+H)

### 2) Synthesis of Compound CPD 60-3

Referring to the synthesis and purification method of compound CPD 8-7, it was only necessary to change the corresponding raw materials to obtain a target compound CPD 60-3 (35.16 g, purity: 99.21%, yield: 76.04%), mass spectrum: 445.31 (M+H)

### 3) Synthesis of Compound CPD 60

Referring to the synthesis and purification method of compound CPD 34, it was only necessary to change the corresponding raw materials to obtain a target compound CPD 60 (21.96 g, purity: 99.95%, yield: 76.37%). 21.96 g of the crude CPD 60 were purified by sublimation to obtain a sublimated pure CPD 60 (17.81 g, purity: 99.95%, yield: 81.11%), mass spectrum: 626.22 (M+H). ¹H NMR (400 MHz, CDCl₃) δ 8.14 (d, J=7.9 Hz, 1H), 8.12-8.04 (m, 2H), 8.00-7.95 (m, 1H), 7.95-7.91 (m, 2H), 7.72-7.66 (m, 2H), 7.60-7.37 (m, 12H), 7.35-7.29 (m, 1H), 2.08-2.03 (m, 2H), 1.95-1.90 (m, 2H), 1.53-1.49 (m, 2H).

### Example 4

In this example, a compound (recorded as CPD 65) was prepared, and its preparation route was as follows:

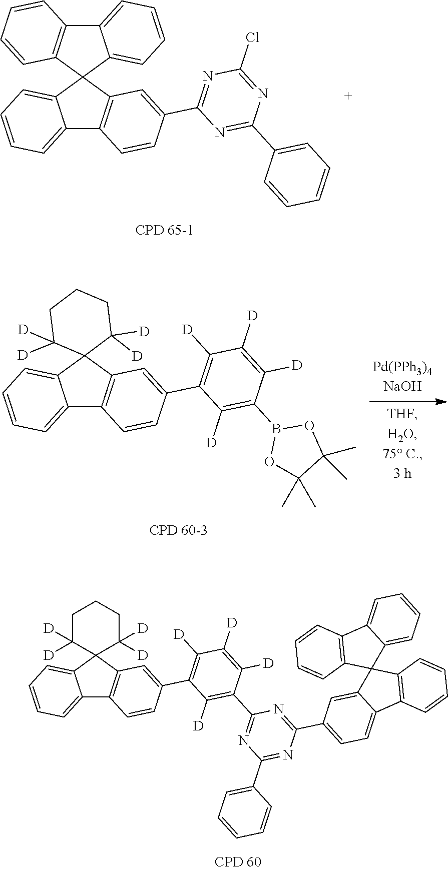 [SMILES: Clc1nc(-c2ccccc2)nc(-c2ccc3c(c2)C2(c4ccccc4-c4ccccc42)c2ccccc2-3)n1 ; [2H]c1c([2H])c(B2OC(C)(C)C(C)(C)O2)c([2H])c(-c2ccc3c(c2)C2(c4ccccc4-3)C([2H])([2H])CCCC2([2H])[2H])c1[2H] ; [2H]c1c([2H])c(-c2ccc3c(c2)C2(c4ccccc4-3)C([2H])([2H])CCCC2([2H])[2H])c([2H])c(-c2nc(-c3ccccc3)nc(-c3ccc4c(c3)C3(c5ccccc5-c5ccccc53)c3ccccc3-4)n2)c1[2H]]

### 1) Synthesis of Compound CPD 65

Referring to the synthesis and purification method of compound CPD 34, it was only necessary to change the corresponding raw materials to obtain the target compound CPD 65 (25.14 g, purity: 99.93%, yield: 78.08%). 25.14 g of the crude CPD 65 were purified by sublimation to obtain a sublimated pure CPD 65 (19.63 g, purity: 99.93%, yield: 78.09%), mass spectrum: 788.31 (M+H). ¹H NMR (400 MHz, CDCl₃) δ 8.35 (d, J=2.0 Hz, 1H), 8.24 (d, J=9.5 Hz, 1H), 8.14 (d, J=7.9 Hz, 1H), 8.12-8.06 (m, 2H), 8.06-7.95 (m, 8H), 7.56-7.48 (m, 8H), 7.48-7.37 (m, 5H), 7.33-7.31 (m, 1H), 2.08-2.03 (m, 2H), 1.95-1.90 (m, 2H), 1.53-1.49 (m, 2H).

### 2) Synthesis of Compound CPD 75

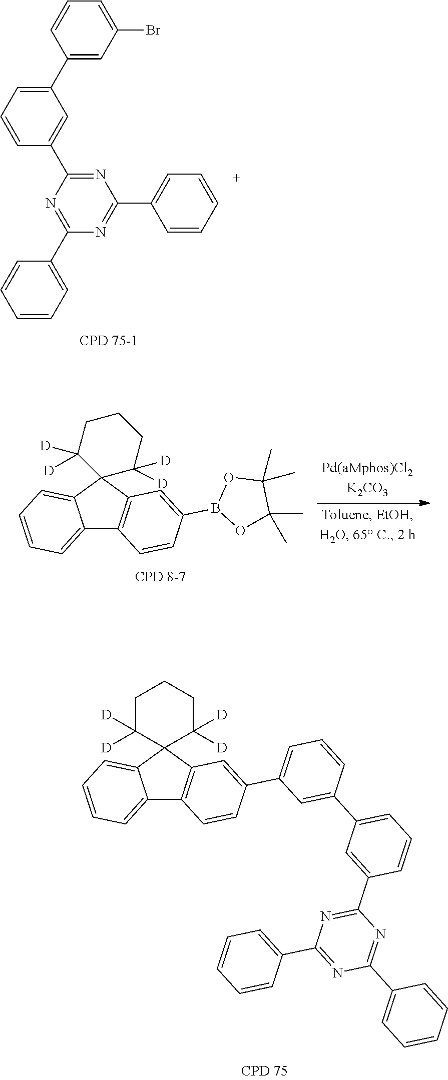 [SMILES: Brc1cccc(-c2cccc(-c3nc(-c4ccccc4)nc(-c4ccccc4)n3)c2)c1 ; [2H]C1([2H])CCCC([2H])([2H])C12c1ccccc1-c1ccc(B3OC(C)(C)C(C)(C)O3)cc12 ; [2H]C1([2H])CCCC([2H])([2H])C12c1ccccc1-c1ccc(-c3cccc(-c4cccc(-c5nc(-c6ccccc6)nc(-c6ccccc6)n5)c4)c3)cc12]

### Example 5

In this example, a compound (recorded as CPD 75) was prepared, and its preparation route was as follows:

Referring to the synthesis and purification method of compound CPD 8, it was only necessary to change the corresponding raw materials to obtain a target compound CPD 75 (28.05 g, purity: 99.96%, yield: 78.88%). 28.05 g of the crude CPD 75 were purified by sublimation to obtain a sublimated pure CPD 75 (21.79 g, purity: 99.96%, yield: 77.69%), mass spectrum: 622.32 (M+H). CDCl₃ was used as a deuterated reagent to measure the hydrogen spectrum, the results were as follows: ¹HNMR (400 MHz, CDCl₃) 9.07-8.06 (m, 1H), 8.82-8.80 (m, 5H), 8.01-8.00 (m, 1H), 7.96-7.91 (m, 2H), 7.86 (d, J=7.8 Hz, 1H), 7.81-7.79 (m, 1H), 7.76-7.68 (m, 5H), 7.66-7.57 (m, 7H), 7.40-7.38 (m, 1H), 7.36-7.34 (m, 1H), 1.96 (t, J=5.9 Hz, 4H), 1.83-1.79 (m, 2H).

### Example 6

In this example, a compound (recorded as CPD 81) was prepared, and its preparation route was as follows:

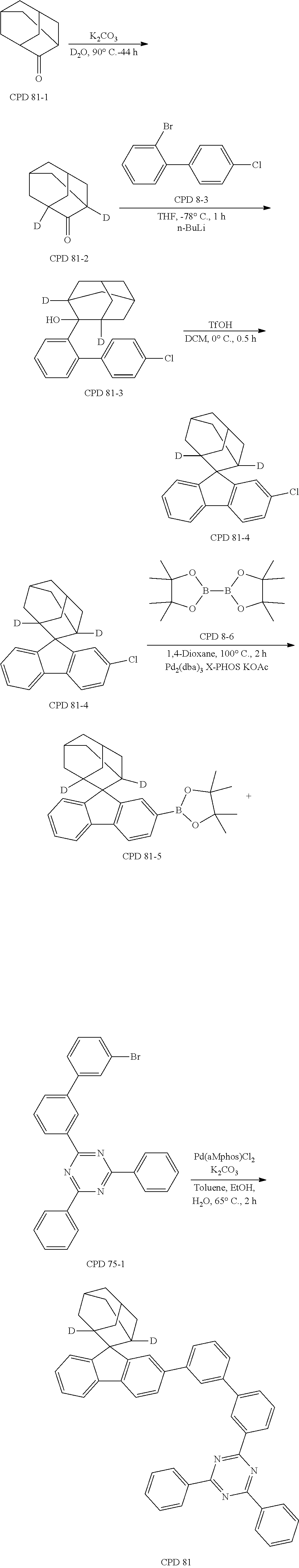

### 1) Synthesis of Compound CPD 81-2

Referring to the synthesis and purification method of compound CPD 8-2, it was only necessary to change the corresponding raw materials to obtain a target compound CPD 81-2 (32.54 g, purity: 99.87%, the deuteration content of four D was 97.55%, yield: 85.63%), mass spectrum: 152.12 (GC-MS).

### 2) Synthesis of Compound CPD 81-3

Referring to the synthesis and purification method of compound CPD 8-4, it was only necessary to change the corresponding raw materials to obtain a target compound CPD 81-3 (35.05 g, purity: 99.20%, yield: 78.65%), mass spectrum: 341.12 (M+H).

### 3) Synthesis of Compound CPD 81-4

Referring to the synthesis and purification method of compound CPD 8-5, it was only necessary to change the corresponding raw materials to obtain a target compound CPD 81-4 (25.09 g, purity: 99.56%, yield: 75.62%), mass spectrum: 323.15 (M+H).

### 4) Synthesis of Compound CPD 81-5

Referring to the synthesis and purification method of compound CPD 8-7, it was only necessary to change the corresponding raw materials to obtain a target compound CPD 81-5 (22.58 g, purity: 99.32%, yield: 77.62%), mass spectrum: 415.06 (M+H).

### 5) Synthesis of Compound CPD 81

Referring to the synthesis and purification method of compound CPD 8, it was only necessary to change the corresponding raw materials to obtain a target compound CPD 81 (18.65 g, purity: 99.95%, yield: 73.23%). 18.65 g of the crude CPD 81 were purified by sublimation to obtain a sublimated pure CPD 81 (14.92 g, purity: 99.95%, yield: 80.00%), mass spectrum: 672.32 (M+H). ¹H NMR (400 MHz, CDCl₃) δ 8.29 (d, J=7.7 Hz, 1H), 8.21 (t, J=2.1 Hz, 1H), 8.13-8.04 (m, 4H), 7.95-7.94 (m, 1H), 7.89-7.83 (m, 2H), 7.78 (dd, J=7.9, 2.2 Hz, 1H), 7.71 (t, J=2.2 Hz, 1H), 7.66-7.63 (m, 1H), 7.60-7.39 (m, 12H), 7.33-7.26 (m, 1H), 2.21-2.18 (m, 4H), 1.96-1.87 (m, 6H), 1.67-1.65 (m, 2H).

### Example 7

In this example, a compound (recorded as CPD 96) was prepared, and its preparation route was as follows:

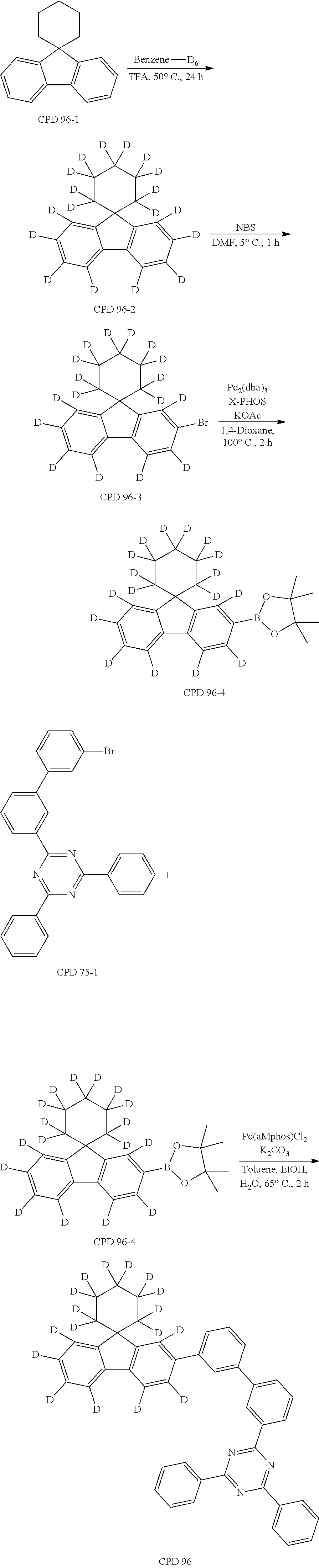

### 1) Synthesis of Compound CPD 96-2

The compound CPD 96-1 (30.00 g, 128.02 mmol), deuterated benzene-D6 (269.32 g, 3.2 mol), and trifluoroacetic acid (14.60 g, 128.02 mmol) were added into a 1,000 mL single-necked round-bottomed flask, pumping vacuum and purging with nitrogen gas for three times. Then the system was heated to 50° C. and stirred for 24 h. The system was cooled to room temperature and added with heavy water (30 mL) to quench the reaction, stirred for 0.5 h, added with ethyl acetate (300 mL) and washed in deionized water (200 mL×3) three times. The combined organic phase was concentrated under reduced pressure at 65° C. for 1 h to obtain a white solid. The white solid was mixed with silica gel and put on the column by dry method for purification by silica gel column chromatography (200-300 mesh silica gel, n-hexane=100% as the eluent). After elution, the purified solution was concentrated under reduced pressure at 75° C. for 1 h to obtain a white solid as CPD 98-2 (29.10 g, purity: 99.51%. For the calculation method of HR-MS deuteration content, please refer to the patent document CN115266981B. The deuteration content of 18 D was 97.33%, yield: 90.04%), mass spectrum: 253.22 (M+H).

### 2) Synthesis of Compound CPD 96-3

Compound CPD 96-2 (27.00 g, 106.95 mmol) and N,N-dimethylformamide (405 mL) were added to a 1,000 mL single-necked round-bottomed flask, pumping vacuum and purging with nitrogen gas for three times. Then the system was cooled to 5° C. and N-bromosuccinimide (NBS) (19.04 g, 106.95 mmol was added in batches over 10 min and stirred for 1 h at 5° C. TLC (n-hexane=100% as the developing agent) monitored that the raw material CPD 96-2 was consumed completely. Deionized water was added to quench the reaction (1,000 mL), and the system was stirred at room temperature for 1 h, and a large amount of solid precipitated. The solid was filtered by suction to obtain a filter cake, and the filter cake was washed once with in deionized water (300 mL) to obtain a white wet solid. The obtained white wet solid was mixed with silica gel and put on the column by dry method for purification by silica gel column chromatography (200-300 mesh silica gel, n-hexane=100% as the eluent). After elution, the purified solution was concentrated under reduced pressure at 75° C. for 1 h to obtain a white solid as CPD 96-3 (30.22 g, purity: 99.82%, yield: 85.54%), mass spectrum: 330.12 (M+H).

### 3) Synthesis of Compound CPD 96-4

Referring to the synthesis and purification method of compound CPD 8-7, it was only necessary to change the corresponding raw materials to obtain a target compound CPD 96-4 (25.82 g, purity: 99.06%, yield: 78.06%), mass spectrum: 378.32 (M+H).

### 4) Synthesis of Compound CPD 96

Referring to the synthesis and purification method of compound CPD 8, it was only necessary to change the corresponding raw materials to obtain a target compound CPD 96 (25.88 g, purity: 99.94%, yield: 76.05%). 25.88 g of the crude CPD 96 were purified by sublimation to obtain a sublimated pure CPD 96 (20.66 g, purity: 99.94%, yield: 79.83%), mass spectrum: 635.40 (M+H). ¹H NMR (400 MHz, CDCl₃) δ 8.21 (t, J=2.1 Hz, 1H), 8.12-8.03 (m, 4H), 7.87-7.85 (m, 1H), 7.74-7.72 (m, 1H), 7.67-7.58 (m, 2H), 7.58-7.47 (m, 8H), 7.47-7.39 (m, 1H).

### Example 8

In this example, a compound (recorded as CPD 126) was prepared, and its preparation route was follows:

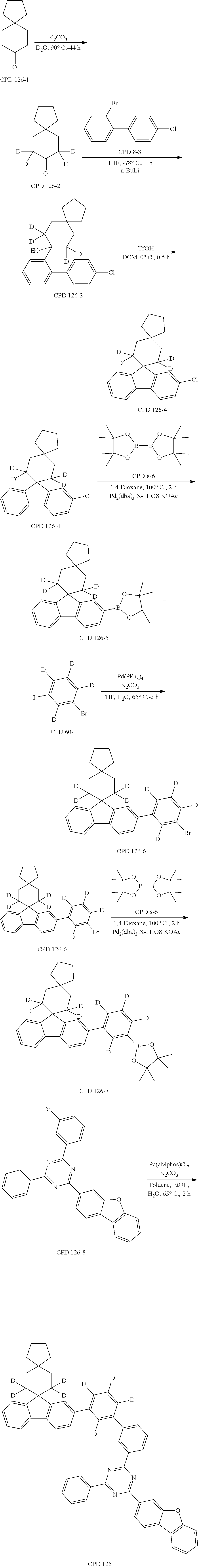

### 1) Synthesis of Compound CPD 126-2

Referring to the synthesis and purification method of compound CPD 8-2, it was only necessary to change the corresponding raw materials to obtain a target compound CPD 126-2 (35.06 g, purity: 99.85%, the deuteration content of four D was 97.05%, yield: 84.85%), mass spectrum: 156.14 (GC-MS).

### 2) Synthesis of Compound CPD 126-3

Referring to the synthesis and purification method of compound CPD 8-4, it was only necessary to change the corresponding raw materials to obtain a target compound CPD 126-3 (30.05 g, purity: 99.16%, yield: 80.06%), mass spectrum: 345.18 (M+H).

### 3) Synthesis of Compound CPD 126-4

Referring to the synthesis and purification method of compound CPD 8-5, it was only necessary to change the corresponding raw materials to obtain a target compound CPD 126-4 (26.85 g, purity: 99.52%, yield: 76.51%), mass spectrum: 327.18 (M+H).

### 4) Synthesis of Compound CPD 126-5

Referring to the synthesis and purification method of compound CPD 8-7, it was only necessary to change the corresponding raw materials to obtain a target compound CPD 126-5 (22.09 g, purity: 99.00%, yield: 74.66%), mass spectrum: 419.30 (M+H).

### 5) Synthesis of Compound CPD 126-6

Referring to the synthesis and purification method of compound CPD 8-10, it was only necessary to change the corresponding raw materials to obtain a target compound CPD 126-6 (18.56 g, purity: 99.74%, yield: 76.59%), mass spectrum: 451.18 (M+H).

### 6) Synthesis of Compound CPD 126-7

Referring to the synthesis and purification method of compound CPD 8-7, it is necessary to change the corresponding raw materials to obtain a target compound CPD 126-7 (19.00 g, purity: 99.03%, yield: 77.38%), mass spectrum: 499.32 (M+H).

### 7) Synthesis of Compound CPD 126

Referring to the synthesis and purification method of compound CPD 8, it was only necessary to change the corresponding raw materials to obtain a target compound CPD 126 (20.88 g, purity: 99.95%, yield: 77.74%). 20.88 g of the crude CPD 126 were purified by sublimation to obtain a sublimated pure CPD 126 (16.30 g, purity: 99.95%, yield: 78.07%), mass spectrum: 770.40 (M+H). ¹H NMR (400 MHz, CDCl₃) δ 8.16-8.04 (m, 5H), 8.00-7.89 (m, 3H), 7.87-7.85 (m, 1H), 7.72 (d, J=2.2 Hz, 1H), 7.67-7.60 (m, 1H), 7.60-7.37 (m, 11H), 7.35-7.29 (m, 1H), 2.65 (s, 4H), 1.49 (s, 8H).

### Example 9

In this example, a compound (recorded as CPD 142) was prepared, and its preparation route was as follows:

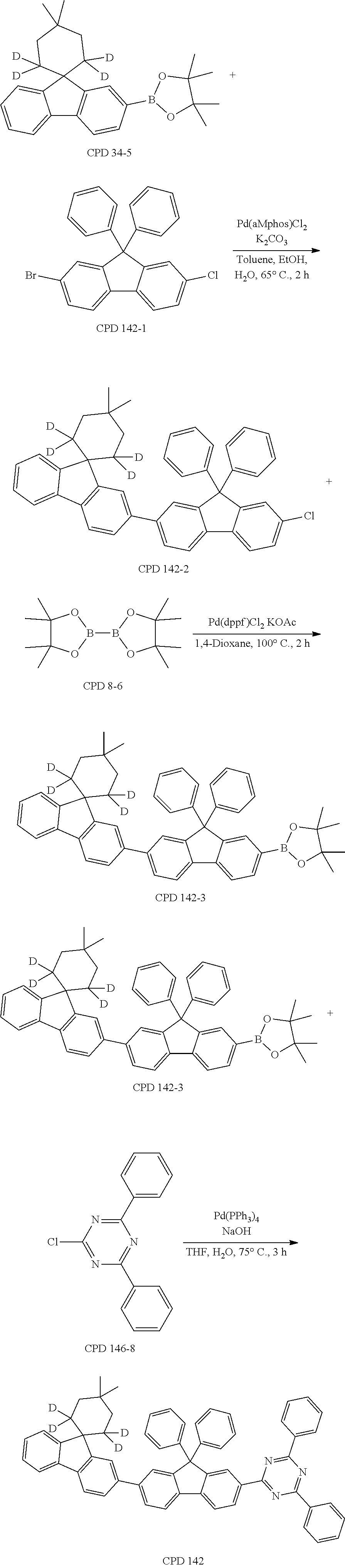

### 1) Synthesis of Compound CPD 142-2

Referring to the synthesis and purification method of compound CPD 8, it was only necessary to change the corresponding raw materials to obtain a target compound CPD 142-2 (22.11 g, purity: 99.34%, yield: 75.96%), mass spectrum: 617.22 (M+H).

### 2) Synthesis of Compound CPD 142-3

Referring to the synthesis and purification method of compound CPD 146-7, it was only necessary to change the corresponding raw materials to obtain a target compound CPD 142-3 (18.88 g, purity: 98.06%, yield: 75.74%), mass spectrum: 709.42 (M+H).

### 3) Synthesis of Compound CPD 142

Referring to the synthesis and purification method of compound CPD 34, it was only necessary to change the corresponding raw materials to obtain a target compound CPD 142 (15.63 g, purity: 99.94%, yield: 76.85%). 15.63 g of the crude CPD 142 were purified by sublimation to obtain a sublimated pure CPD 142 (12.55 g, purity: 99.94%, yield: 80.30%), mass spectrum: 814.40 (M+H).

1H NMR (400 MHz, CDCl₃) δ 8.45 (d, J=2.2 Hz, 1H), 8.35-8.30 (m, 1H), 8.16 (d, J=7.7 Hz, 1H), 8.10-7.96 (m, 8H), 7.82-7.75 (m, 3H), 7.54-7.41 (m, 12H), 7.34-7.28 (m, 7H), 2.56 (s, 4H). 0.84 (s, 6H).

### Example 10

In this example, a compound (recorded as CPD 146) was prepared, and its preparation route was as follows:

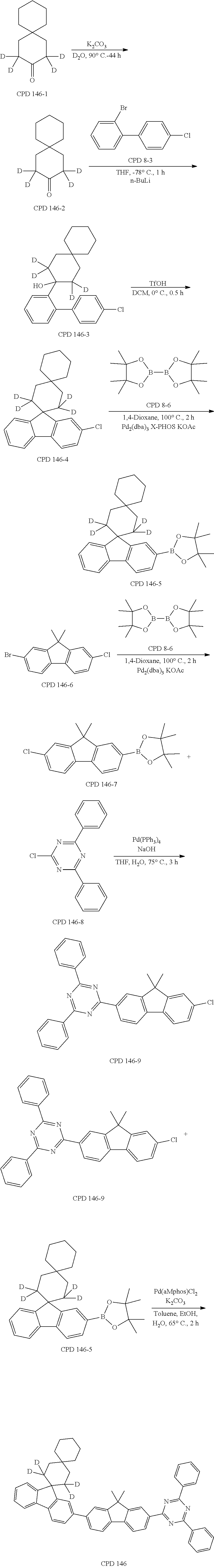

### 1) Synthesis of Compound CPD 146-2

Referring to the synthesis and purification method of compound CPD 8-2, it was only necessary to change the corresponding raw materials to obtain a target compound CPD 146-2 (34.52 g, purity: 99.85%, the deuteration content of four D was 97.66%, yield: 84.62%), mass spectrum: 170.16 (GC-MS).

### 2) Synthesis of Compound CPD 146-3

Referring to the synthesis and purification method of compound CPD 8-4, it was only necessary to change the corresponding raw materials to obtain a target compound CPD 146-3 (28.87 g, purity: 99.62%, yield: 80.63%), mass spectrum: 359.20 (M+H).

### 3) Synthesis of Compound CPD 146-4

Referring to the synthesis and purification method of compound CPD 8-5, it was only necessary to change the corresponding raw materials to obtain a target compound CPD 146-4 (22.54 g, purity: 99.45%, yield: 78.52%), mass spectrum: 341.22 (M+H).

### 4) Synthesis of Compound CPD 146-5

Referring to the synthesis and purification method of compound CPD 8-7, it was only necessary to change the corresponding raw materials to obtain a target compound CPD 146-5 (20.65 g, purity: 99.01%, yield: 74.96%), mass spectrum: 433.32 (M+H).

### 5) Synthesis of Compound CPD 146-7

CPD 146-6 (25.00 g, 81.27 mmol), CPD 8-6 (24.77 g, 97.52 mmol), 1,1-bis(diphenylphosphine)diphenyliron palladium dichloride (1.18 g, 1.62 mmol), potassium acetate (15.95 g, 162.54 mmol), and 1,4-dioxane (375 mL) were added into a 1,000 mL three-necked round-bottomed flask, pumping vacuum and purging with nitrogen gas for three times, and then the system was heated to 100° C. to react for 2 h. TLC (ethyl acetate:n-hexane=1:10 as the developing agent) monitored that the raw material CPD 146-6 was consumed completely. The system was cooled to 60° C., concentrated under reduced pressure to remove the solvent, added with ethyl acetate (600 mL), and washed twice with deionized water (200 mL×2). The obtain liquids were separated and mixed with silica gel and put on the column by dry method for purification by silica gel column chromatography (200-300 mesh silica gel, ethyl acetate:n-hexane=1:15 as the eluent). After elution, the purified solution was concentrated under reduced pressure at 70° C. for 1 h to obtain a white solid as CPD 146-7 (22.65 g, purity: 98.89%, yield: 78.57%), mass spectrum: 355.16 (M+H).

### 6) Synthesis of Compound CPD 146-9

Referring to the synthesis and purification method of compound CPD 34, it was only necessary to change the corresponding raw materials to obtain a target compound CPD 146-9 (23.33 g, purity: 99.72%, yield: 77.68%), mass spectrum: 460.12 (M+H).

### 7) Synthesis of Compound CPD 146

Referring to the synthesis and purification method of compound CPD 8, it was only necessary to change the corresponding raw materials to obtain a target compound CPD 146 (19.81 g, purity: 99.96%, yield: 78.03%). 19.81 g of the crude CPD 146 was purified by sublimation to obtain a sublimated pure CPD 146 (15.45 g, purity: 99.96%, yield: 77.99%), mass spectrum: 730.40 (M+H). ¹H NMR (400 MHz, CDCl₃) δ 8.22 (d, J=1.9 Hz, 1H), 8.16 (d, J=7.7 Hz, 1H), 8.12 (d, J=2.1 Hz, 1H), 8.1-8.05 (m, 4H), 8.02-7.93 (m, 4H), 7.83-7.76 (m, 3H), 7.53-7.44 (m, 8H), 7.33-7.31 (m, 1H), 2.65 (s, 4H), 1.80 (s, 6H), 1.57-1.51 (m, 4H), 1.51-1.43 (m, 4H), 1.43-1.35 (m, 2H).

### Example 11

In this example, a compound (recorded as CPD 161) was prepared, and its preparation route was as follows:

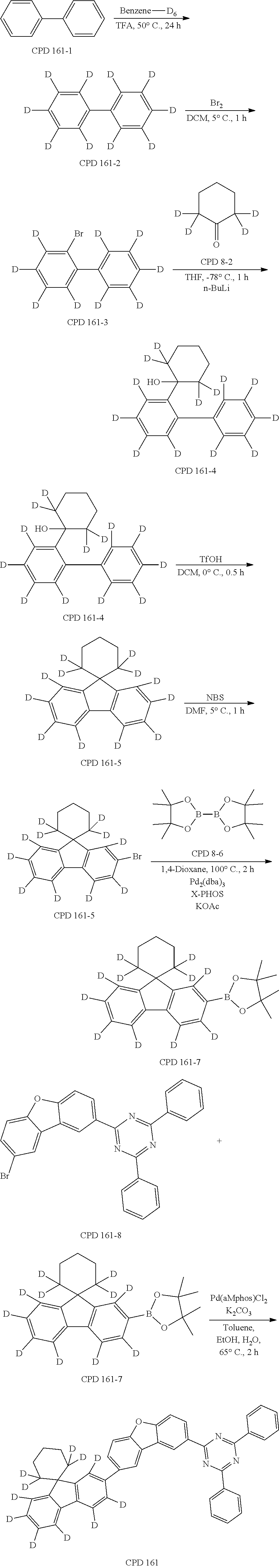

### 1) Synthesis of Compound CPD 161-2

Referring to the synthesis and purification method of compound CPD 96-2, it was only necessary to change the corresponding raw materials to obtain a target compound CPD 161-2 (30.52 g, purity: 99.63%. The calculation method of HR-MS deuteration content can be found in the patent document CN115266981B. The deuteration content of 10 D was 97.42%, yield: 90.26%), mass spectrum: 165.14 (M+H).

### 2) Synthesis of Compound CPD 161-3

The compound CPD 161-2 (28.00 g, 170.45 mmol) and methylene chloride (420 mL) were added to a 1,000 mL three-necked round-bottomed flask, pumping vacuum and purging with nitrogen gas for three times, and then the system was cooled to 5° C., then added with bromine (28.60 g, 178.97 mmol) dropwise over10 min and stirred for 1 h at 5° C. TLC (n-hexane=100% as the developing agent) monitored that the raw material CPD 161-2 was consumed completely. A 10% (mass fraction) sodium bisulfite aqueous solution was added dropwise to the system to quench the reaction (100 mL). After rising to room temperature, the liquids were separated directly. The organic phase was washed twice with deionized water (200 mL×2), and the combined organic phase was concentrated under reduced pressure at 65° C. for 1 h to obtain a white solid. The white solid was mixed with silica gel and put onto the column by dry method for purification by silica gel column chromatography (200-300 mesh silica gel, n-hexane=100% as the eluent). After elution, the purified solution was concentrated under reduced pressure at 75° C. for 1 h to obtain a white solid as CPD 161-3 (35.91 g, purity: 99.87%, yield: 87.00%), mass spectrum: 242.05 (M+H).

### 3) Synthesis of Compound CPD 161-4

Referring to the synthesis and purification method of compound CPD 8-4, it was only necessary to change the corresponding raw materials to obtain a target compound CPD 161-4 (28.45 g, purity: 99.33%, yield: 81.63%), mass spectrum: 266.23 (M+H).

### 4) Synthesis of Compound CPD 161-5

Referring to the synthesis and purification method of compound CPD 8-5, it was only necessary to change the corresponding raw materials to obtain a target compound CPD 161-5 (20.21 g, purity: 99.56%, yield: 78.86%), mass spectrum: 247.23 (M+H).

### 5) Synthesis of Compound CPD 161-6

Referring to the synthesis and purification method of compound CPD 96-3, it was only necessary to change the corresponding raw materials to obtain a target compound CPD 161-6 (19.74 g, purity: 99.59%, yield: 75.00%), mass spectrum: 324.14 (M+H).

### 6) Synthesis of Compound CPD 161-7

Referring to the synthesis and purification method of compound CPD 8-7, it was only necessary to change the corresponding raw materials to obtain a target compound CPD 161-7 (16.65 g, purity: 99.02%, yield: 76.53%), mass spectrum: 372.30 (M+H).

### 7) Synthesis of Compound CPD 161

Referring to the synthesis and purification method of compound CPD 8, it was only necessary to change the corresponding raw materials to obtain a target compound CPD 161 (19.06 g, purity: 99.95%, yield: 74.66%). 19.06 g of crude CPD 161 were purified by sublimation to obtain a sublimated pure CPD 161 (15.22 g, purity: 99.96%, yield: 79.85%), mass spectrum: 643.22 (M+H). ¹H NMR (400 MHz, CDCl₃) δ 8.19 (d, J=2.4 Hz, 1H), 8.12-8.04 (m, 4H), 7.87-7.79 (m, 2H), 7.60-7.45 (m, 9H), 2.19-2.14 (m, 2H), 2.06-2.01 (m, 2H), 1.54-1.48 (m, 2H).

### Example 12

In this example, a compound (recorded as CPD 167) was prepared, and its preparation route was as follows:

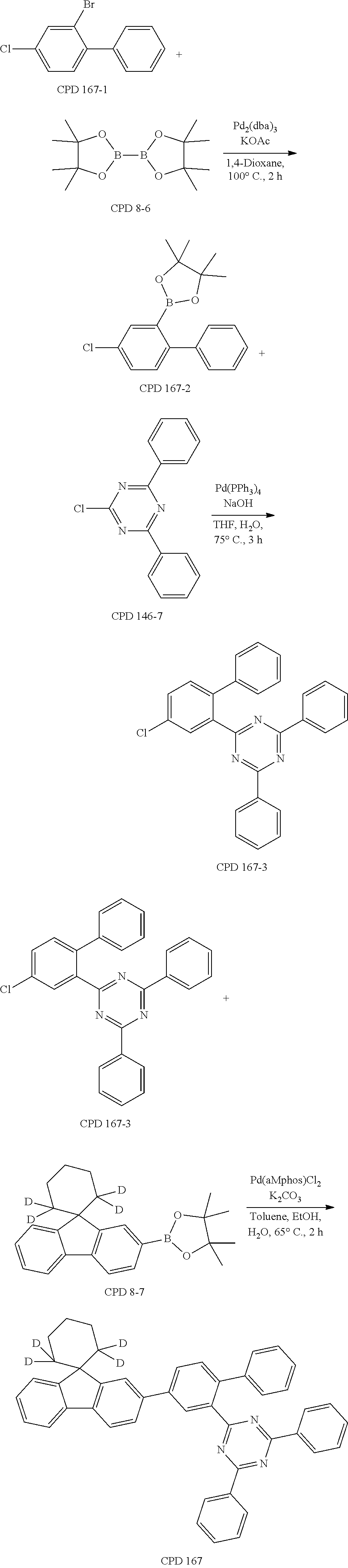

### 1) Synthesis of Compound CPD 167-2

Referring to the synthesis and purification method of compound CPD 146-6, it was only necessary to change the corresponding raw materials to obtain a target compound CPD 167-2 (35.55 g, purity: 99.31%, yield: 78.53%), mass spectrum: 315.14 (M+H).

### 2) Synthesis of Compound CPD 167-3

Referring to the synthesis and purification method of compound CPD 34, it was only necessary to change the corresponding raw materials to obtain a target compound CPD 167-3 (32.46 g, purity: 99.66%, yield: 76.01%), mass spectrum: 420.12 (M+H).

### 3) Synthesis of Compound CPD 167

Referring to the synthesis and purification method of compound CPD 8, it is necessary to change the corresponding raw materials to obtain a target compound CPD 167 (25.33 g, purity: 99.96%, yield: 79.84%). 25.33 g of the crude CPD 167 were purified by sublimation to obtain a sublimated pure CPD 167 (20.80 g, purity: 99.96%, yield: 82.11%), mass spectrum: 622.32 (M+H). ¹H NMR (400 MHz, CDCl₃) δ 8.16 (d, J=7.7 Hz, 1H), 8.12-8.04 (m, 4H), 7.98-7.97 (m, 1H), 7.83-7.75 (m, 3H), 7.65-7.58 (m, 2H), 7.54-7.51 (m, 3H), 7.50-7.46 (m, 7H), 7.44-7.36 (m, 3H), 7.33-7.31 (m, 1H), 2.08-2.03 (m, 2H), 1.95-1.90 (m, 2H), 1.53-1.48 (m, 2H).

### Example 13

In this example, a compound (recorded as CPD 177) was prepared, and its preparation route was as follows:

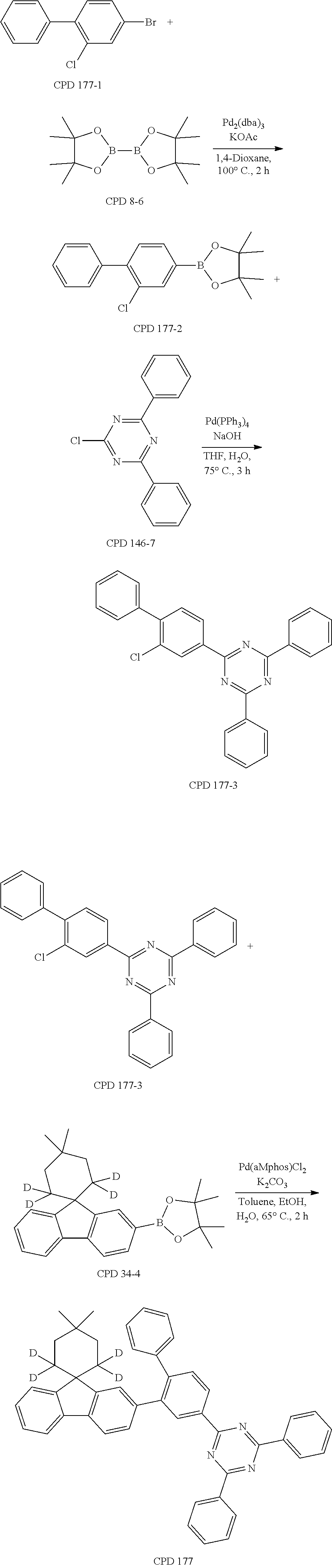

### 1) Synthesis of Compound CPD 177-2

Referring to the synthesis and purification method of compound CPD 146-6, it was only necessary to change the corresponding raw materials to obtain a target compound CPD 177-2 (34.98 g, purity: 99.20%, yield: 77.53%), mass spectrum: 315.14 (M+H).

### 2) Synthesis of Compound CPD 177-3

Referring to the synthesis and purification method of compound CPD 34, it was only necessary to change the corresponding raw materials to obtain a target compound CPD 177-3 (30.52 g, purity: 99.68%, yield: 75.96%), mass spectrum: 420.12 (M+H).

### 3) Synthesis of Compound CPD 177

Referring to the synthesis and purification method of compound CPD 8, it was only necessary to change the corresponding raw materials to obtain a target compound CPD 177 (22.11 g, purity: 99.94%, yield: 78.44%). 22.11 g of the crude CPD 177 were purified by sublimation to obtain a sublimated pure CPD 177 (17.70 g, purity: 99.94%, yield: 80.05%), mass spectrum: 650.34 (M+H). ¹H NMR (400 MHz, CDCl₃) δ 8.20 (d, J=7.6 Hz, 1H), 8.12-8.04 (m, 4H), 8.00-7.96 (m, 1H), 7.94 (d, J=2.3 Hz, 1H), 7.90 (d, J=2.2 Hz, 1H), 7.85-7.82 (m, 1H), 7.77-7.70 (m, 2H), 7.55-7.44 (m, 10H), 7.44-7.38 (m, 3H), 7.35-7.29 (m, 1H), 2.56 (s, 4H), 0.86 (s, 6H).

### Example 14

In this example, a compound (recorded as CPD 182) was prepared, and its preparation route was as follows:

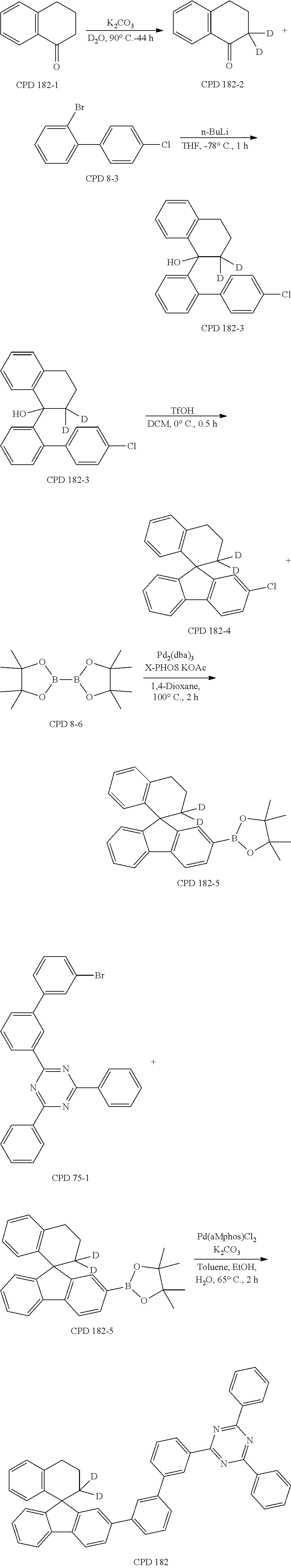

### 1) Synthesis of Compound CPD 182-2

Referring to the synthesis and purification method of compound CPD 8-2, it was only necessary to change the corresponding raw materials to obtain a target compound CPD 182-2 (40.33 g, purity: 99.79%, the deuteration content of two D was 98.02%, yield: 87.78%), mass spectrum: 148.08 (GC-MS).

### 2) Synthesis of Compound CPD 182-3

Referring to the synthesis and purification method of compound CPD 8-4, it was only necessary to change the corresponding raw materials to obtain a target compound CPD 182-3 (32.01 g, purity: 99.33%, yield: 56.86%), mass spectrum: 337.12 (M+H).

### 3) Synthesis of Compound CPD 182-4

Referring to the synthesis and purification method of compound CPD 8-5, it was only necessary to change the corresponding raw materials to obtain a target compound CPD 182-4 (28.09 g, purity: 99.63%, yield: 77.63%), mass spectrum: 319.22 (M+H).

### 4) Synthesis of Compound CPD 182-5

Referring to the synthesis and purification method of compound CPD 8-7, it was only necessary to change the corresponding raw materials to obtain a target compound CPD 182-5 (22.63 g, purity: 98.88%, yield: 72.11%), mass spectrum: 411.02 (M+H).

### 5) Synthesis of Compound CPD 182

Referring to the synthesis and purification method of compound CPD 8, it was only necessary to change the corresponding raw materials to obtain a target compound CPD 182 (16.85 g, purity: 99.95%, yield: 77.05%). 16.85 g of the crude CPD 182 were purified by sublimation to obtain a sublimated pure CPD 182 (13.01 g, purity: 99.95%, yield: 77.21%), mass spectrum: 668.30 (M+H).

1H NMR (400 MHz, CDCl₃) δ 8.38 (d, J=7.9 Hz, 1H), 8.24-8.16 (m, 2H), 8.12-8.03 (m, 4H), 8.03-7.97 (m, 1H), 7.87-7.85 (m, 1H), 7.78 (dd, J=7.8, 2.1 Hz, 1H), 7.71 (t, J=2.2 Hz, 1H), 7.67-7.61 (m, 1H), 7.60-7.51 (m, 5H), 7.51-7.47 (m, 8H), 7.36-7.34 (m, 1H), 7.31-7.28 (m, 1H), 7.24-7.21 (m, 1H), 7.18-7.16 (m, 1H), 2.91-2.77 (m, 2H), 2.38-2.33 (m, 1H), 2.25-2.20 (m, 1H).

### Example 15

In this example, a compound (recorded as CPD 184) was prepared, and its preparation route was as follows:

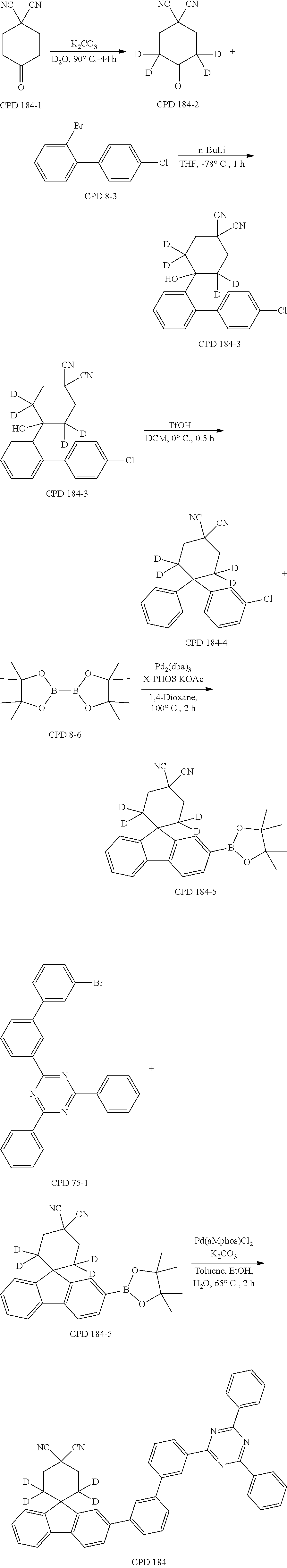

### 1) Synthesis of Compound CPD 184-2

Referring to the synthesis and purification method of compound CPD 8-2, it was only necessary to change the corresponding raw materials to obtain a target compound CPD 184-2 (28.96 g, purity: 99.85%, the deuteration content of four D was 97.66%, yield: 88.06%), mass spectrum: 152.09 (GC-MS).

### 2) Synthesis of Compound CPD 184-3

Referring to the synthesis and purification method of compound CPD 8-4, it was only necessary to change the corresponding raw materials to obtain a target compound CPD 184-3 (24.33 g, purity: 99.56%, yield: 57.68%), mass spectrum: 341.12 (M+H).

### 3) Synthesis of Compound CPD 184-4

Referring to the synthesis and purification method of compound CPD 8-5, it was only necessary to change the corresponding raw materials to obtain a target compound CPD 184-4 (20.69 g, purity: 99.75%, yield: 78.64%), mass spectrum: 323.22 (M+H).

### 4) Synthesis of Compound CPD 184-5

Referring to the synthesis and purification method of compound CPD 8-7, it was only necessary to change the corresponding raw materials to obtain a target compound CPD 184-5 (20.41 g, purity: 98.06%, yield: 75.61%), mass spectrum: 415.22 (M+H).

### 5) Synthesis of Compound CPD 184

Referring to the synthesis and purification method of compound CPD 8, it was only necessary to change the corresponding raw materials to obtain a target compound CPD 184 (17.11 g, purity: 99.95%, yield: 76.85%). 17.11 g of crude CPD 184 were purified by sublimation to obtain a sublimated pure CPD 184 (13.56 g, purity: 99.95%, yield: 79.26%), mass spectrum: 672.32 (M+H). 1H NMR (400 MHz, CDCl₃) δ 8.21 (t, J=2.1 Hz, 1H), 8.16 (d, J=7.7 Hz, 1H), 8.12-8.04 (m, 4H), 7.98 (dd, J=6.5, 1.8 Hz, 1H), 7.87-7.85 (m, 1H), 7.83-7.75 (m, 2H), 7.71 (t, J=2.2 Hz, 1H), 7.65-7.63 (m, 1H), 7.60-7.44 (m, 12H), 7.33-7.31 (m, 1H), 2.53 (s, 4H).

### Example 16

In this example, a compound (recorded as CPD 185) was prepared, and its preparation route was as follows:

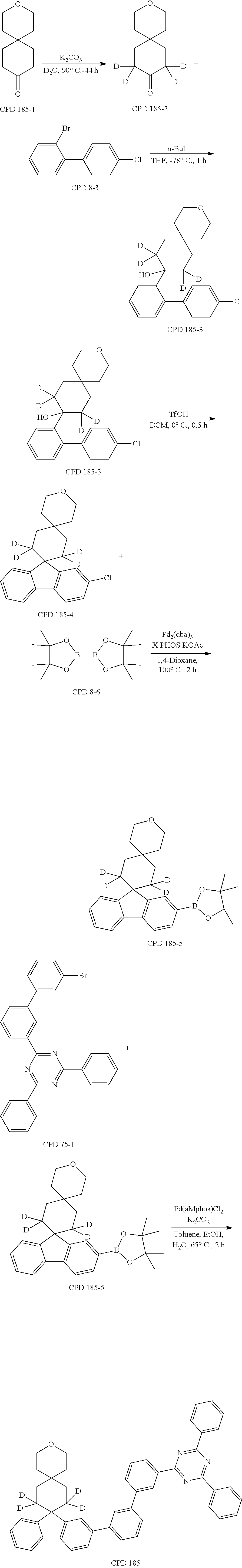

### 1) Synthesis of Compound CPD 185-2

Referring to the synthesis and purification method of compound CPD 8-2, it was only necessary to change the corresponding raw materials to obtain a target compound CPD 185-2 (25.66 g, purity: 99.80%, the deuteration content of four D was 97.12%, yield: 87.52%), mass spectrum: 172.04 (GC-MS).

### 2) Synthesis of Compound CPD 185-3

Referring to the synthesis and purification method of compound CPD 8-4, it was only necessary to change the corresponding raw materials to obtain a target compound CPD 185-3 (22.71 g, purity: 99.56%, yield: 60.55%), mass spectrum: 361.22 (M+H).

### 3) Synthesis of Compound CPD 185-4

Referring to the synthesis and purification method of compound CPD 8-5, it was only necessary to change the corresponding raw materials to obtain a target compound CPD 185-4 (18.08 g, purity: 99.68%, yield: 78.64%), mass spectrum: 343.02 (M+H).

### 4) Synthesis of Compound CPD 185-5

Referring to the synthesis and purification method of compound CPD 8-7, it was only necessary to change the corresponding raw materials to obtain a target compound CPD 185-5 (17.65 g, purity: 98.65%, yield: 76.06%), mass spectrum: 435.24 (M+H).

### 5) Synthesis of Compound CPD 185

Referring to the synthesis and purification method of compound CPD 8, it was only necessary to change the corresponding raw materials to obtain a target compound CPD 185 (14.52 g, purity: 99.93%, yield: 77.85%). 14.52 g of the crude CPD 185 were purified by sublimation to obtain a sublimated pure CPD 185 (11.83 g, purity: 99.95%, yield: 81.48%), mass spectrum: 692.34 (M+H).

1H NMR (400 MHz, CDCl₃) δ 8.21 (t, J=2.1 Hz, 1H), 8.16 (d, J=7.7 Hz, 1H), 8.12-8.04 (m, 4H), 8.00-7.95 (m, 1H), 7.87-7.85 (m, 1H), 7.83-7.75 (m, 2H), 7.71 (t, J=2.2 Hz, 1H), 7.65-7.63 (m, 1H), 7.60-7.55 (m, 3H), 7.55-7.44 (m, 9H), 7.33-7.31 (m, 1H), 3.69-3.63 (m, 4H), 2.51 (s, 4H), 1.63-1.57 (m, 4H).

### Example 17

In this example, a compound (recorded as CPD 189) was prepared, and its preparation route was as follows:

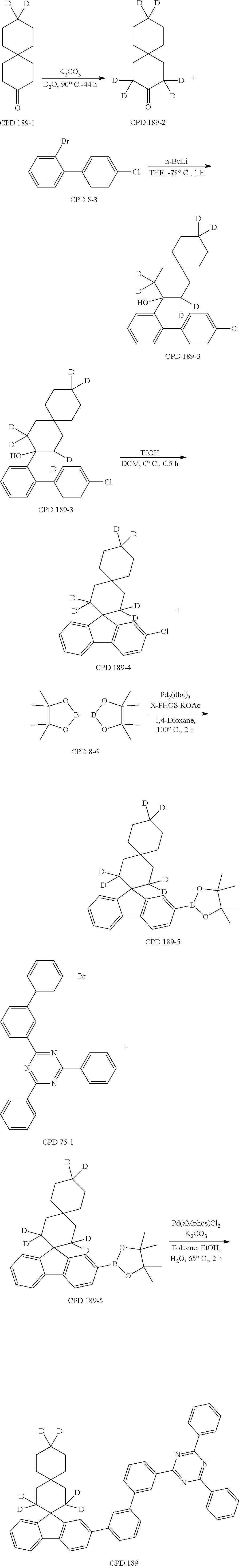

### 1) Synthesis of Compound CPD 189-2

Referring to the synthesis and purification method of compound CPD 8-2, it was only necessary to change the corresponding raw materials to obtain a target compound CPD 189-2 (20.02 g, purity: 99.80%, the deuteration content of six D was 97.12%, yield: 88.02%), mass spectrum: 172.16 (GC-MS).

### 2) Synthesis of Compound CPD 189-3

Referring to the synthesis and purification method of compound CPD 8-4, it was only necessary to change the corresponding raw materials to obtain a target compound CPD 189-3 (17.33 g, purity: 99.51%, yield: 61.55%), mass spectrum: 361.21 (M+H).

### 3) Synthesis of Compound CPD 189-4

Referring to the synthesis and purification method of compound CPD 8-5, it is necessary to change the corresponding raw materials to obtain a target compound CPD 189-4 (14.33 g, purity: 99.52%, yield: 77.02%), mass spectrum: 343.22 (M+H).

### 4) Synthesis of Compound CPD 189-5

Referring to the synthesis and purification method of compound CPD 8-7, it is necessary to change the corresponding raw materials to obtain a target compound CPD 189-5 (13.33 g, purity: 98.98%, yield: 77.056%), mass spectrum: 435.20 (M+H).

### 5) Synthesis of Compound CPD 189

Referring to the synthesis and purification method of compound CPD 8, it was only necessary to change the corresponding raw materials to obtain a target compound CPD 189 (11.56 g, purity: 99.93%, yield: 75.63%). 11.56 g of the crude CPD 189 were purified by sublimation to obtain a sublimated pure CPD 189 (8.88 g, purity: 99.93%, yield: 76.82%), mass spectrum: 692.32 (M+H).

1H NMR (400 MHz, CDCl₃) δ 8.21 (t, J=2.1 Hz, 1H), 8.16 (d, J=7.7 Hz, 1H), 8.12-8.04 (m, 4H), 8.00-7.95 (m, 1H), 7.86-7.84 (m, 1H), 7.83-7.75 (m, 2H), 7.71 (t, J=2.2 Hz, 1H), 7.64-7.63 (m, 1H), 7.60-7.44 (m, 12H), 7.33-7.31 (m, 1H), 2.65 (s, 4H), 1.78-1.72 (m, 4H), 1.48-1.42 (m, 4H).

### Comparative Example 1

This example provided a compound that was commercially available and had the following structural formula:

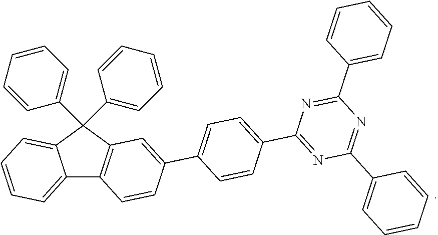 [SMILES: c1ccc(-c2nc(-c3ccccc3)nc(-c3ccc(-c4ccc5c(c4)C(c4ccccc4)(c4ccccc4)c4ccccc4-5)cc3)n2)cc1]

### Comparative Example 2

This example provided a compound that was commercially available and had the following structural formula:

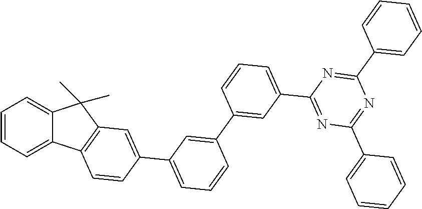 [SMILES: CC1(C)c2ccccc2-c2ccc(-c3cccc(-c4cccc(-c5nc(-c6ccccc6)nc(-c6ccccc6)n5)c4)c3)cc21]

### Comparative Example 3

This example provided a compound that was commercially available and had the following structural formula:

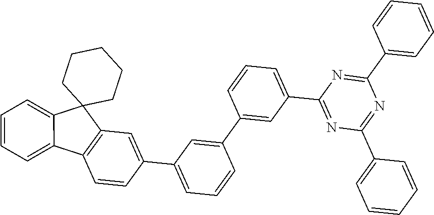 [SMILES: c1ccc(-c2nc(-c3ccccc3)nc(-c3cccc(-c4cccc(-c5ccc6c(c5)C5(CCCCC5)c5ccccc5-6)c4)c3)n2)cc1]

### Comparative Example 4

This example provided a compound that was commercially available and had the following structural formula:

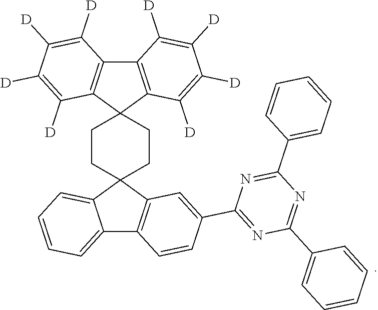 [SMILES: [2H]c1c([2H])c([2H])c2c(c1[2H])-c1c([2H])c([2H])c([2H])c([2H])c1C21CCC2(CC1)c1ccccc1-c1ccc(-c3nc(-c4ccccc4)nc(-c4ccccc4)n3)cc12]

### Application Example

The compounds prepared in the above examples and the compounds in the comparative examples were applied to the production of an organic electroluminescent device. Specifically, as shown in FIG. 1, the organic electroluminescent device included stacked glass substrate 1, anode 2, hole injection layer 3, first hole transport layer (HTL1) 4, second hole transport layer (HTL2) 5, light emitting layer 6, hole blocking layer (HBL) 7, electron transport layer 8 (ETL) and cathode 9.

The preparation method of the above-mentioned organic electroluminescent device included:

A 50 mm×50 mm×1.0 mm glass substrate with an ITO (100 nm) transparent electrode (anode 2) was ultrasonically cleaned in ethanol for 10 min, dried at 150° C. and treated with N₂ Plasma for 30 min. The washed glass substrate was mounted on a substrate holder of a vacuum evaporation device. First, the compound HATCN was evaporated on the side of the transparent electrode line to cover the transparent electrode, forming a thin film with a thickness of 5 nm (hole injection layer 3), followed by evaporation of a layer of compound HTM1 to form a thin film with a thickness of 60 nm as HTL1 (first hole transport layer 4), then a layer of compound HTM2 was evaporated on the compound HTM1 film to form a film with a thickness of 10 nm as HTL2 (second hole transport layer 5), and then the host material compound and the guest material compound (a weight ratio of the host material compound and the guest (doped) material compound was 98%: 2%) were evaporated on the HTM2 film layer in a co-evaporation mode, forming a light-emitting layer with a film thickness of 25 nm. A hole blocking layer (HBL) with a thickness of 5 nm and an electron transport layer with a thickness of 350 nm were sequentially formed on the light-emitting layer by evaporation method. The hole blocking layer was formed from the compounds or the comparative compounds. The electron transport layer was formed form the electron transport layer material ETL or the compound of the embodiments of the present disclosure or the comparative examples: LiQ (weight ratio 1:1). Then, Mg/Ag (100 nm, 1:9) were evaporated as the cathode material in the co-evaporation mode.

The structural formulas of the above-mentioned compounds HATCN, HTM1, HTM2, host material compound, guest material compound, ETL, and LiQ were as follows:

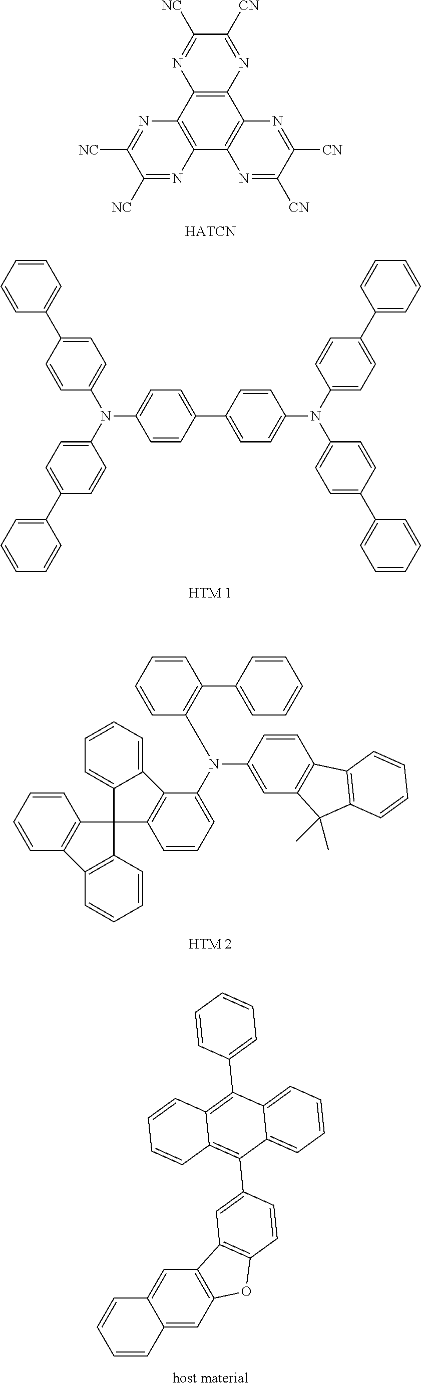

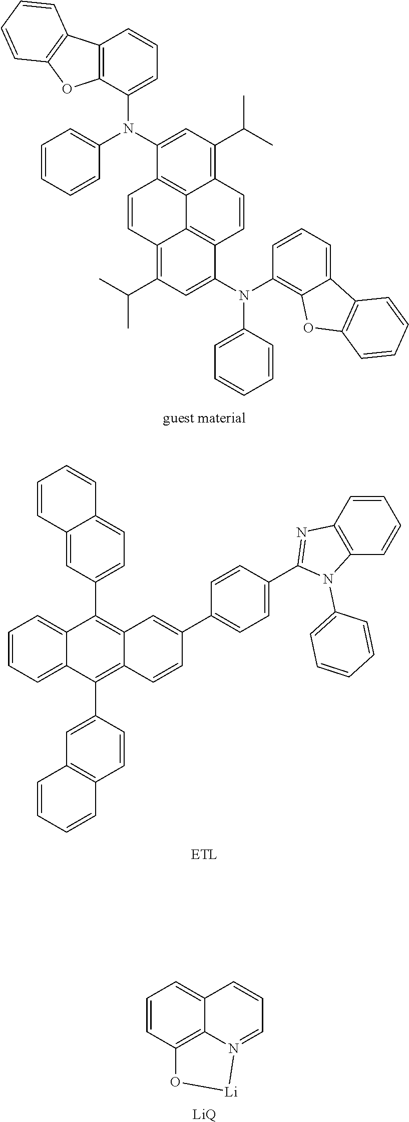

### Application Effect Test:

The above devices were subjected to performance testing. In each of the examples and comparative examples, a fixed current density was allowed to flow through the light-emitting element through a constant current power supply (Keithley 2400), and the luminescence spectrum was tested by a spectroradiometer (CS 2000). In addition, when the current density was 10 mA/cm², the device voltage value, efficiency and the time for the test brightness to reach 95% of the initial brightness (LT95) were measured. The results were shown in Table 1 below:

TABLE 1

| | Compound | Compound | | Current | |
|---|---|---|---|---|---|
| | (hole | (electron | Starting | Effi- | LT95(h) |
| | blocking | transport | Voltage | ciency | @1,000 |
| | layer) | layer) | (V) | (Cd/A) | nits |
| Application | CPD 8 | ETL:LiQ | 3.71 | 8.08 | 173 |
| Example 1 | | | | | |
| Application | CPD 34 | ETL:LiQ | 3.68 | 8.11 | 180 |
| Example 2 | | | | | |
| Application | CPD 60 | ETL:LiQ | 3.74 | 8.24 | 216 |
| Example 3 | | | | | |
| Application | CPD 65 | ETL:LiQ | 3.81 | 8.09 | 198 |
| Example 4 | | | | | |
| Application | CPD 75 | ETL:LiQ | 3.73 | 8.16 | 202 |
| Example 5 | | | | | |
| Application | CPD 81 | ETL:LiQ | 3.84 | 8.19 | 221 |
| Example 6 | | | | | |
| Application | CPD 96 | ETL:LiQ | 3.80 | 8.34 | 237 |
| Example 7 | | | | | |
| Application | CPD 126 | ETL:LiQ | 3.77 | 8.17 | 194 |
| Example 8 | | | | | |
| Application | CPD 142 | ETL:LiQ | 3.81 | 8.23 | 187 |
| Example 9 | | | | | |
| Application | CPD 146 | ETL:LiQ | 3.82 | 8.29 | 186 |
| Example 10 | | | | | |
| Application | CPD 161 | ETL:LiQ | 3.83 | 8.31 | 205 |
| Example 11 | | | | | |
| Application | CPD 167 | ETL:LiQ | 3.71 | 8.42 | 203 |
| Example 12 | | | | | |
| Application | CPD 177 | ETL:LiQ | 3.70 | 8.38 | 211 |
| Example 13 | | | | | |
| Application | CPD 182 | ETL:LiQ | 3.79 | 8.32 | 204 |
| Example 14 | | | | | |
| Application | CPD 184 | ETL:LiQ | 3.68 | 8.09 | 241 |
| Example 15 | | | | | |
| Application | CPD 185 | ETL:LiQ | 3.73 | 8.12 | 194 |
| Example 16 | | | | | |
| Application | CPD 189 | ETL:LiQ | 3.75 | 8.27 | 224 |
| Example 17 | | | | | |
| Application | Comparative | CPD 75:LiQ | 3.78 | 8.12 | 191 |
| Example 18 | Example 1 | | | | |
| | compound | | | | |
| Application | Comparative | CPD 65:LiQ | 3.74 | 8.32 | 194 |
| Example 19 | Example 2 | | | | |
| | compound | | | | |
| Application | CPD 81 | CPD 34:LiQ | 3.76 | 8.36 | 204 |
| Example 20 | | | | | |
| Application | CPD 96 | CPD 75:LiQ | 3.77 | 8.40 | 192 |
| Example 21 | | | | | |
| Application | Comparative | ETL:LiQ | 3.82 | 7.45 | 93 |
| comparative | Example 1 | | | | |
| example 1 | compound | | | | |
| Application | Comparative | ETL:LiQ | 3.94 | 8.03 | 130 |
| comparative | Example 2 | | | | |
| example 2 | compound | | | | |
| Application | Comparative | ETL:LiQ | 3.82 | 8.06 | 136 |
| comparative | Example 3 | | | | |
| example 3 | compound | | | | |
| Application | Comparative | ETL:LiQ | 3.95 | 7.53 | 89 |
| comparative | Example 4 | | | | |
| example 4 | Compound | | | | |
| Application | Comparative | Comparative | 3.91 | 7.42 | 95 |
| comparative | Example 1 | example 1 | | | |
| example 5 | compound | compound:LiQ | | | |
| Application | Comparative | Comparative | 3.94 | 7.95 | 118 |
| comparative | Example 2 | example 2 | | | |
| example 6 | compound | compound:LiQ | | | |

As can be seen from Table 1, the use of the compounds of the present disclosure as a hole blocking layer material and/or an electron transport layer material in an organic electroluminescent device exhibited superior performance in the driving voltage, luminous efficiency and device lifetime compared to the compounds of the comparative examples.

The above results showed that the compounds of the present disclosure had the advantages of good optical, electrical and thermal stability, high luminous efficiency, low voltage, long lifetime, etc., and could be used in organic light-emitting devices. The carbon-deuterium bond in the deuterated A-ring compound of the present disclosure had smaller vibration and higher bond energy than the carbon-hydrogen bond, which could effectively improve the stability of the material and was beneficial to increasing the lifetime of the device. In addition, due to its suitable energy level, it has a significant effect on improving the voltage and efficiency of the device. Therefore, the compounds of the present disclosure, especially when used as a hole blocking layer material and an electron transport layer material, had better application prospects in the AMOLED industry.

1. A compound having the following structural formula: 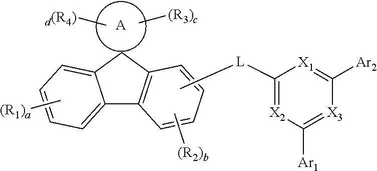
wherein ring A is an aliphatic ring, and the number of ring carbon atoms in the aliphatic ring is e;
X₁, X₂ and X₃ are independently selected from N or CR₀, and at least one of the X₁, X₂ and X₃ is N;
R₀ is selected from hydrogen, deuterium, halogen, cyano, nitro, a substituted or unsubstituted aliphatic hydrocarbyl group with a main chain having ≤40 carbon atoms, a substituted or unsubstituted heteroalkyl group with a main chain having 1-40 carbon atoms, a substituted or unsubstituted alkoxy group with a main chain having 1-40 carbon atoms, a substituted or unsubstituted alkylsilyl group with a main chain having 1-40 carbon atoms, a substituted or unsubstituted alkylboryl group with a main chain having 1-40 carbon atoms, a substituted or unsubstituted alicyclic hydrocarbyl group having 3-40 ring-forming carbon atoms, a substituted or unsubstituted heterocycloalkyl group having 3-40 ring-forming carbon atoms, a substituted or unsubstituted aryl group having 6-60 aromatic ring carbon atoms, a substituted or unsubstituted aryloxy group having 6-60 aromatic ring carbon atoms, a substituted or unsubstituted arylsilyl group having 6-60 aromatic ring carbon atoms, a substituted or unsubstituted arylboryl group having 6-60 aromatic ring carbon atoms, a substituted or unsubstituted arylamine group having 6-60 aromatic ring carbon atoms, a substituted or unsubstituted arylphosphine group having 6-60 aromatic ring carbon atoms, or a substituted or unsubstituted heteroaryl group having 3-60 aromatic ring carbon atoms;
R₁, R₂, and R₃ are each independently selected from deuterium, halogen, cyano, nitro, a substituted or unsubstituted aliphatic hydrocarbyl group with a main chain having ≤40 carbon atoms, a substituted or unsubstituted heteroalkyl group with a main chain having 1-40 carbon atoms, a substituted or unsubstituted alkoxy group with a main chain having 1-40 carbon atoms, a substituted or unsubstituted alkylsilyl group with a main chain having 1-40 carbon atoms, a substituted or unsubstituted alkylboryl group with a main chain having 1-40 carbon atoms, a substituted or unsubstituted alicyclic hydrocarbyl group having 3-40 ring-forming carbon atoms, a substituted or unsubstituted heterocycloalkyl group having 3-40 ring-forming carbon atoms, a substituted or unsubstituted aryl group having 6-60 aromatic ring carbon atoms, a substituted or unsubstituted aryloxy group having 6-60 aromatic ring carbon atoms, a substituted or unsubstituted arylsilyl group having 6-60 aromatic ring carbon atoms, a substituted or unsubstituted arylboryl group having 6-60 aromatic ring carbon atoms, a substituted or unsubstituted arylamine group having 6-60 aromatic ring carbon atoms, a substituted or unsubstituted arylphosphine group having 6-60 aromatic ring carbon atoms, or a substituted or unsubstituted heteroaryl group having 3-60 aromatic ring carbon atoms;
R₄ is selected from deuterium or R₈ group substituted by deuterium;
R₈ is selected from a substituted or unsubstituted aliphatic hydrocarbyl group with a main chain having ≤40 carbon atoms, a substituted or unsubstituted heteroalkyl group with a main chain having 1-40 carbon atoms, a substituted or unsubstituted alkoxy group with a main chain having 1-40 carbon atoms, a substituted or unsubstituted alkylsilyl group with a main chain having 1-40 carbon atoms, a substituted or unsubstituted alkylboryl group with a main chain having 1-40 carbon atoms, a substituted or unsubstituted alicyclic hydrocarbyl group having 3-40 ring-forming carbon atoms, a substituted or unsubstituted heterocycloalkyl group having 3-40 ring-forming carbon atoms, a substituted or unsubstituted aryl group having 6-60 aromatic ring carbon atoms, a substituted or unsubstituted aryloxy group having 6-60 aromatic ring carbon atoms, a substituted or unsubstituted arylsilyl group having 6-60 aromatic ring carbon atoms, a substituted or unsubstituted arylboryl group having 6-60 aromatic ring carbon atoms, a substituted or unsubstituted arylamine group having 6-60 aromatic ring carbon atoms, a substituted or unsubstituted arylphosphine group having 6-60 aromatic ring carbon atoms, or a substituted or unsubstituted heteroaryl group having 3-60 aromatic ring carbon atoms;
a is an integer selected from 0-4; under the condition that a is an integer≥2, adjacent R₁ are connected to each other to form a fused ring or not connected;
b is an integer selected from 0 to 3; under the condition that b is an integer≥2, adjacent R₂ are connected to each other to form a fused ring or not connected;
c is selected from an integer≥0;
d is selected from an integer≥1;
e is selected from an integer≥3;
L is selected from a single bond, a substituted or unsubstituted arylene group, or a substituted or unsubstituted heteroarylene group;
Ar₁ and Ar₂ are independently selected from a substituted or unsubstituted aryl group, or a substituted or unsubstituted heteroaryl group;
heteroatoms in the heteroalkyl group, the heterocycloalkyl group, the heteroaryl group or the heteroarylene group are independently selected from at least one of O, S, N, Se, Si and Ge;
the substituted refers to being substituted by at least one of deuterium, halogen, cyano, isocyano, phosphine group, an alkyl group having 1-6 carbon atoms, a cycloalkyl group having 3-16 ring carbon atoms, an alkyl-substituted amine group having 1-6 carbon atoms, an aryl group having 6-12 carbon atoms, or a deuterated aryl group having 6-12 carbon atoms, wherein the substituted number ranges from a monosubstitution to a maximum substitution.

2. The compound according to claim 1, wherein R₀, R₁, R₂, and R₃ are independently selected from a substituted or unsubstituted aliphatic hydrocarbyl group with a main chain having ≤20 carbon atoms, a substituted or unsubstituted heteroalkyl group with a main chain having 1-20 carbon atoms, a substituted or unsubstituted alkoxy group with a main chain having 1-20 carbon atoms, a substituted or unsubstituted alkylsilyl group with a main chain having 1-20 carbon atoms, a substituted or unsubstituted alkylboryl group with a main chain having 1-20 carbon atoms, a substituted or unsubstituted alicyclic hydrocarbyl group having 3-20 ring-forming carbon atoms, a substituted or unsubstituted heterocycloalkyl group having 3-20 ring-forming carbon atoms, a substituted or unsubstituted aryl group having 6-30 aromatic ring carbon atoms, a substituted or unsubstituted aryloxy group having 6-30 aromatic ring carbon atoms, a substituted or unsubstituted arylsilyl group having 6-30 aromatic ring carbon atoms, a substituted or unsubstituted arylboryl group having 6-30 aromatic ring carbon atoms, a substituted or unsubstituted arylamine group having 6-30 aromatic ring carbon atoms, a substituted or unsubstituted arylphosphine group having 6-30 aromatic ring carbon atoms, or a substituted or unsubstituted heteroaryl group having 3-30 aromatic ring carbon atoms.

3. The compound according to claim 2, wherein R₀, R₁, R₂, and R₃ are independently selected from a substituted or unsubstituted aliphatic hydrocarbyl group with a main chain having ≤10 carbon atoms, a substituted or unsubstituted heteroalkyl group with a main chain having 1-10 carbon atoms, a substituted or unsubstituted alkoxy group with a main chain having 1-10 carbon atoms, a substituted or unsubstituted alkylsilyl group with a main chain having 1-10 carbon atoms, a substituted or unsubstituted alkylboryl group with a main chain having 1-10 carbon atoms, a substituted or unsubstituted alicyclic hydrocarbyl group having 3-10 ring-forming carbon atoms, a substituted or unsubstituted heterocycloalkyl group having 3-10 ring-forming carbon atoms, a substituted or unsubstituted aryl group having 6-12 aromatic ring carbon atoms, a substituted or unsubstituted aryloxy group having 6-12 aromatic ring carbon atoms, a substituted or unsubstituted arylsilyl group having 6-12 aromatic ring carbon atoms, a substituted or unsubstituted arylboryl group having 6-12 aromatic ring carbon atoms, a substituted or unsubstituted arylamine group having 6-12 aromatic ring carbon atoms, a substituted or unsubstituted arylphosphine group having 6-12 aromatic ring carbon atoms, or a substituted or unsubstituted heteroaryl group having 3-12 aromatic ring carbon atoms.

4. The compound according to claim 1, wherein two of X₁, X₂ and X₃ are N, or each of t he X₁, X₂ and X₃ are N; R₁ and R₂ are independently selected from deuterium, halogen, cyano, nitro, an alkyl group with a main chain having 1-10 carbon atoms, an alkoxy group with a main chain having 1-10 carbon atoms, a heteroalkyl group with a main chain having 1-10 carbon atoms, a cycloalkyl group having 3-10 ring-forming carbon atoms, a heterocycloalkyl group having 3-10 ring-forming carbon atoms, an aryl group having 6-12 carbon atoms, an aryloxy group having 6-12 carbon atoms, an arylamine group having 6-12 carbon atoms, an arylphosphine group having 6-12 carbon atoms, or a heteroaryl group having 5-20 carbon atoms; and R₃ is selected from halogen, cyano, nitro, an alkyl group with a main chain having 1-10 carbon atoms, an alkoxy group with a main chain having 1-10 carbon atoms, a heteroalkyl group with a main chain having 1-10 carbon atoms, a cycloalkyl group having 3-10 ring-forming carbon atoms, a heterocycloalkyl group having 3-10 ring-forming carbon atoms, an aryl group having 6-12 carbon atoms, an aryloxy group having 6-12 carbon atoms, an arylamine group having 6-12 carbon atoms, an arylphosphine group having 6-12 carbon atoms, or a heteroaryl group having 3-20 carbon atoms.

5. The compound according to claim 1, wherein 3≤e≤40.

6. The compound according to claim 1, wherein R₁ is deuterium and a is an integer≥1.

7. The compound according to claim 1, wherein R₂ is deuterium and b is an integer≥1.

8. The compound according to claim 1, wherein c is an integer≤8, and d is an integer≤8.

9. The compound according to claim 1, wherein 3≤e≤20.

10. The compound according to claim 1, wherein c and d are independently selected from an integer≤20.

11. The compound according to claim 1, wherein the ring A is selected from at least one of the following structural formulas:

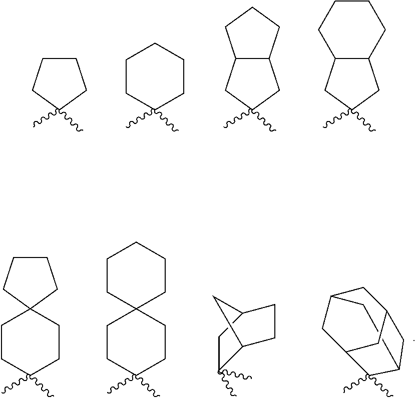

12. The compound according to claim 1, wherein L is selected from a substituted or unsubstituted arylene group having 6-60 carbon atoms, or a substituted or unsubstituted heteroarylene group having 6-60 carbon atoms.

13. The compound according to claim 1, wherein L contains at least one deuterium.

14. The compound according to claim 1, wherein L is selected from at least one of the following structural formulas: 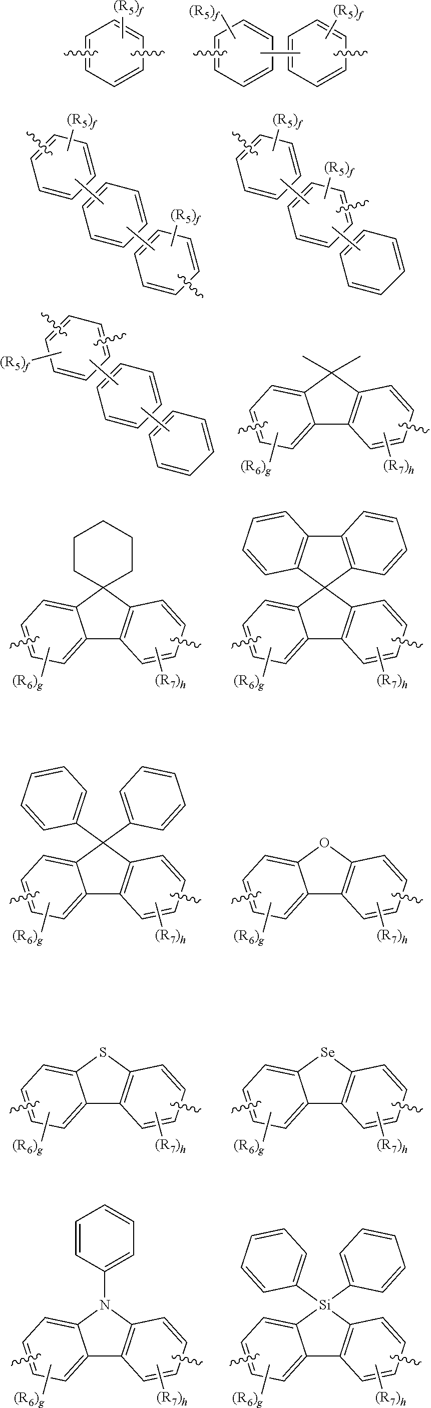
wherein R₅, R₆ and R₇ are independently selected from deuterium, a substituted or unsubstituted alkyl group having 1-20 carbon atoms, a substituted or unsubstituted cycloalkyl group having 3-20 carbon atoms, a substituted or unsubstituted heteroalkyl group having 1-20 carbon atoms, a substituted or unsubstituted heterocycloalkyl group having 3-20 carbon atoms, a substituted or unsubstituted aryl group having 6-20 carbon atoms, or a substituted or unsubstituted heteroaryl group having 3-20 carbon atoms; wherein f, g and h are independently selected from an integer from 0 to 5.

15. The compound according to claim 1, wherein Ar₁ and Ar₂ are independently selected from a substituted or unsubstituted aryl group having 6-30 carbon atoms, or a substituted or unsubstituted heteroaryl group having 2-30 carbon atoms.

16. The compound according to claim 1, wherein Ar₁ and Ar₂ are each independently selected from biphenyl group, naphthyl, anthracenyl, dibenzofuranyl, dibenzothienyl, 9,9-dimethylfluorenyl, 9,9-diphenylfluorenyl, aspirobifluorenyl, phenanthrenyl, pyrenyl, chrysenyl group, carbazolyl, pyridyl, pyrimidinyl, benzophenanthyl, or a substituted or unsubstituted phenyl group.

17. The compound according to claim 1, wherein the arylphosphine group comprises at least one of monoarylphosphine group, diarylphosphine group, and triarylphosphine group; and/or the aliphatic hydrocarbyl group includes at least one of alkyl, alkenyl and alkynyl.

18. A compound selected from one of compounds having the following structural formula:

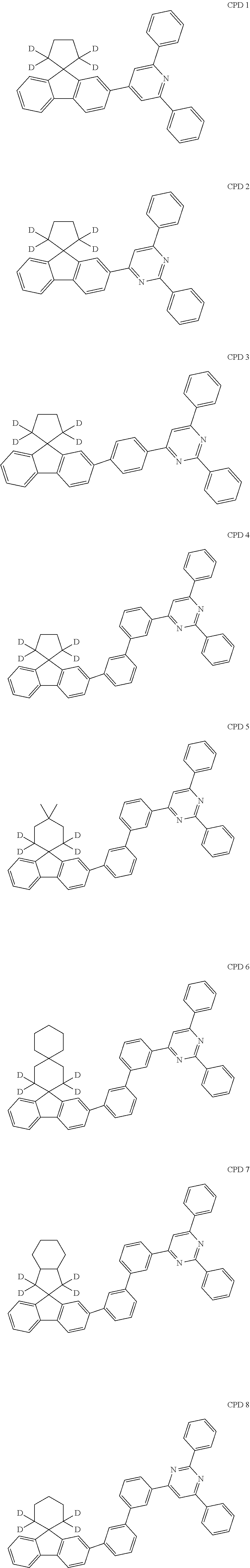

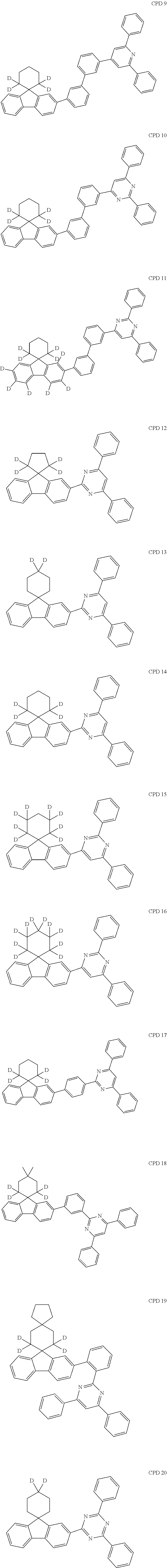

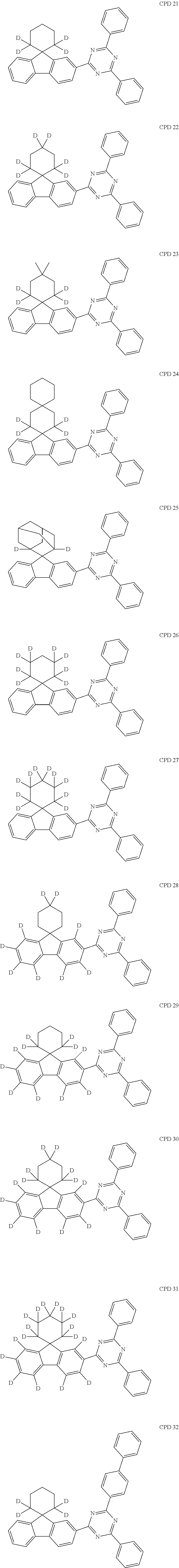

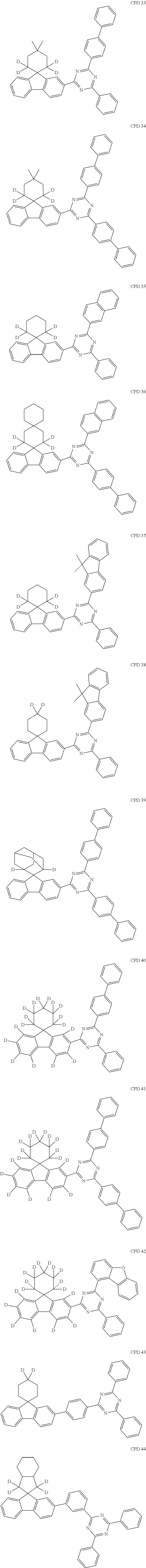

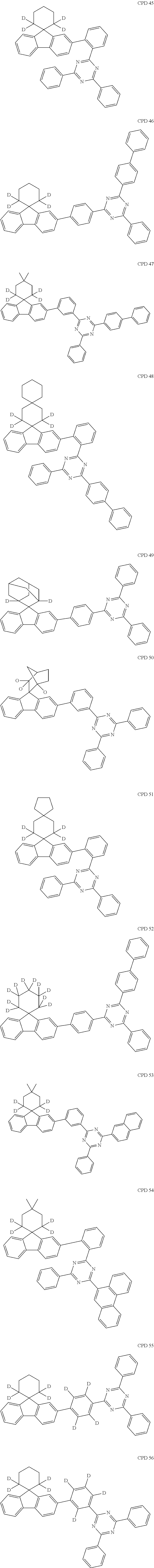

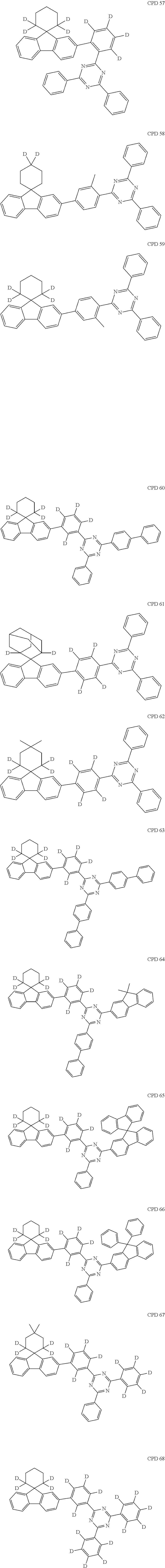

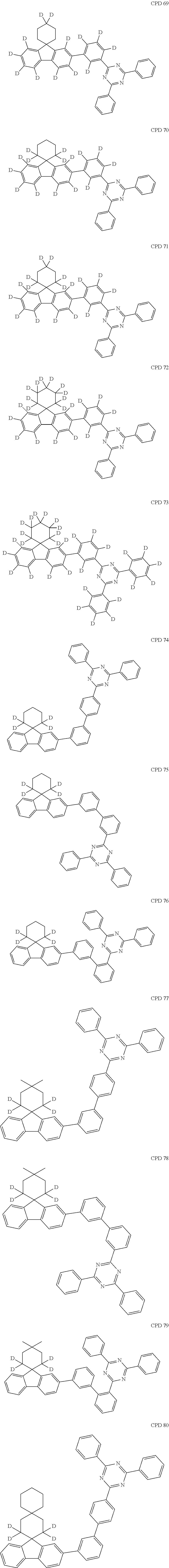

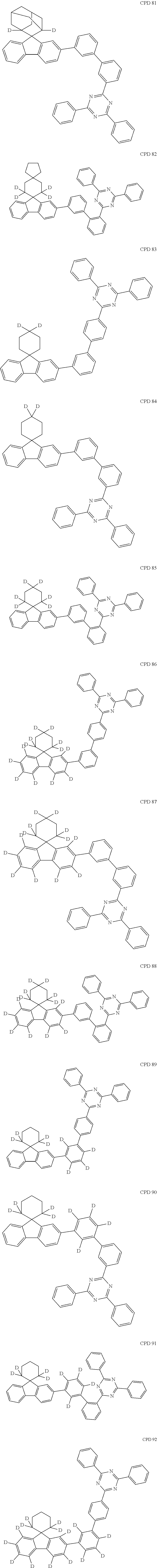

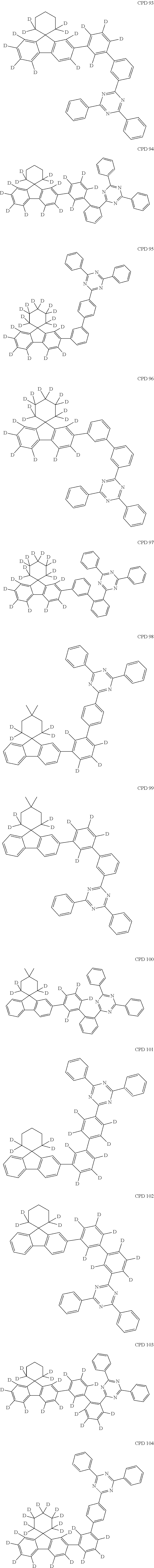

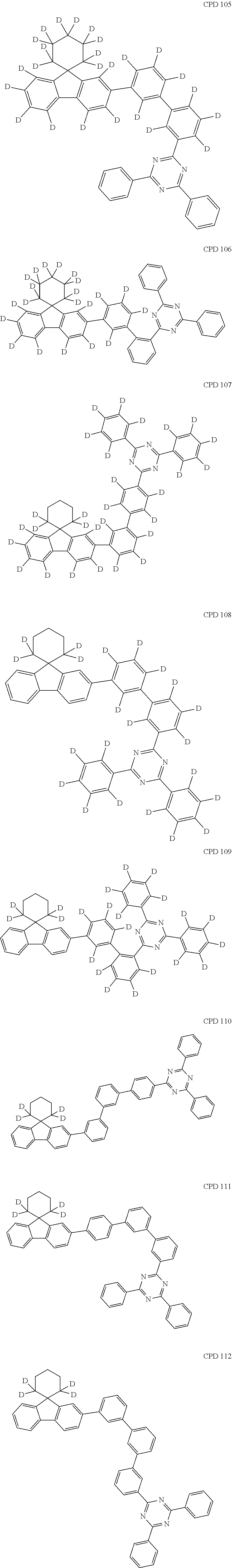

[chemical structure]

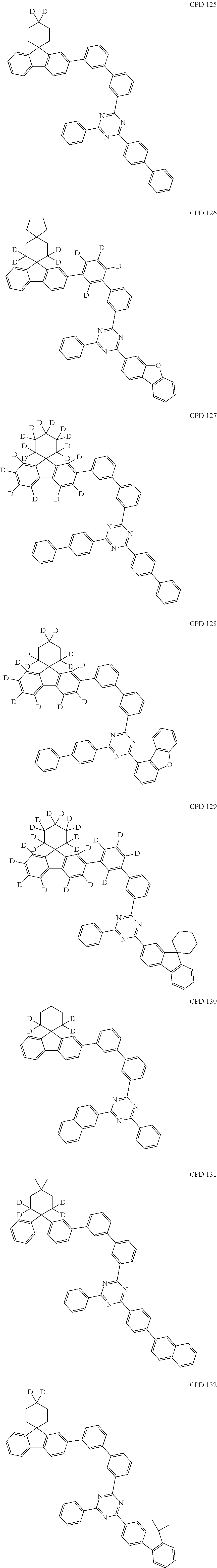

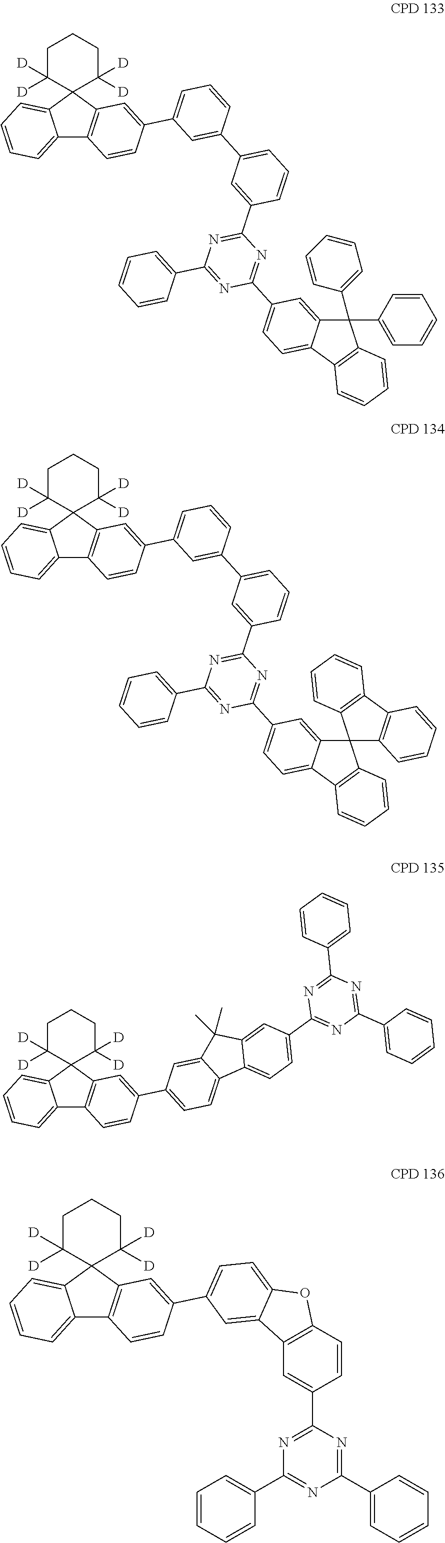

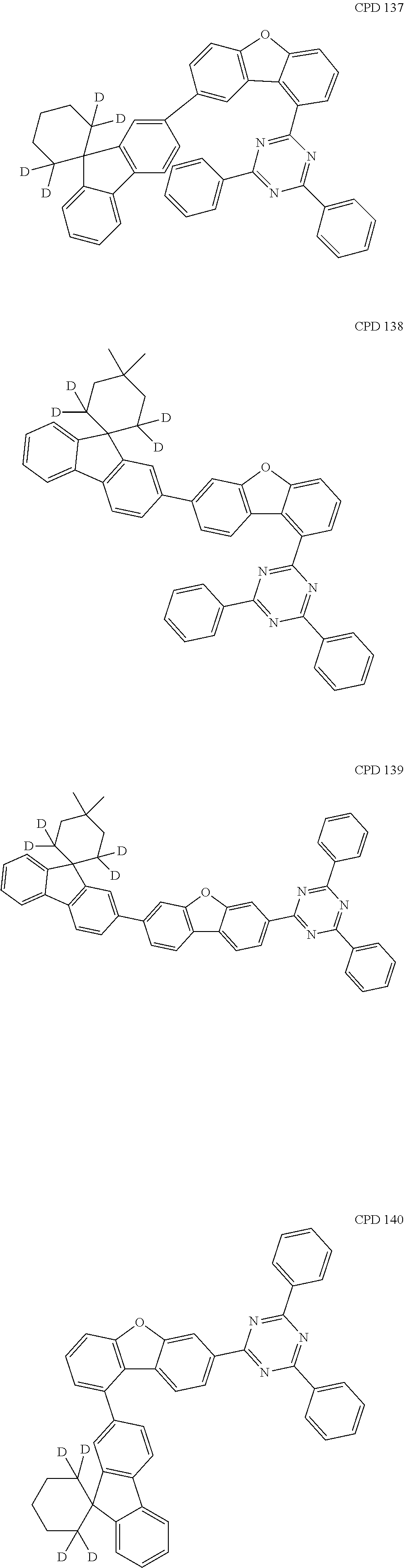 [SMILES: [2H]C1([2H])CCCC([2H])([2H])C12c1ccccc1-c1ccc(-c3ccc4oc5cccc(-c6nc(-c7ccccc7)nc(-c7ccccc7)n6)c5c4c3)cc12 ; [2H]C1([2H])CC(C)(C)CC([2H])([2H])C12c1ccccc1-c1ccc(-c3ccc4c(c3)oc3cccc(-c5nc(-c6ccccc6)nc(-c6ccccc6)n5)c34)cc12 ; [2H]C1([2H])CC(C)(C)CC([2H])([2H])C12c1ccccc1-c1ccc(-c3ccc4c(c3)oc3cc(-c5nc(-c6ccccc6)nc(-c6ccccc6)n5)ccc34)cc12 ; [2H]C1([2H])CCCC([2H])([2H])C12c1ccccc1-c1ccc(-c3cccc4oc5cc(-c6nc(-c7ccccc7)nc(-c7ccccc7)n6)ccc5c34)cc12]

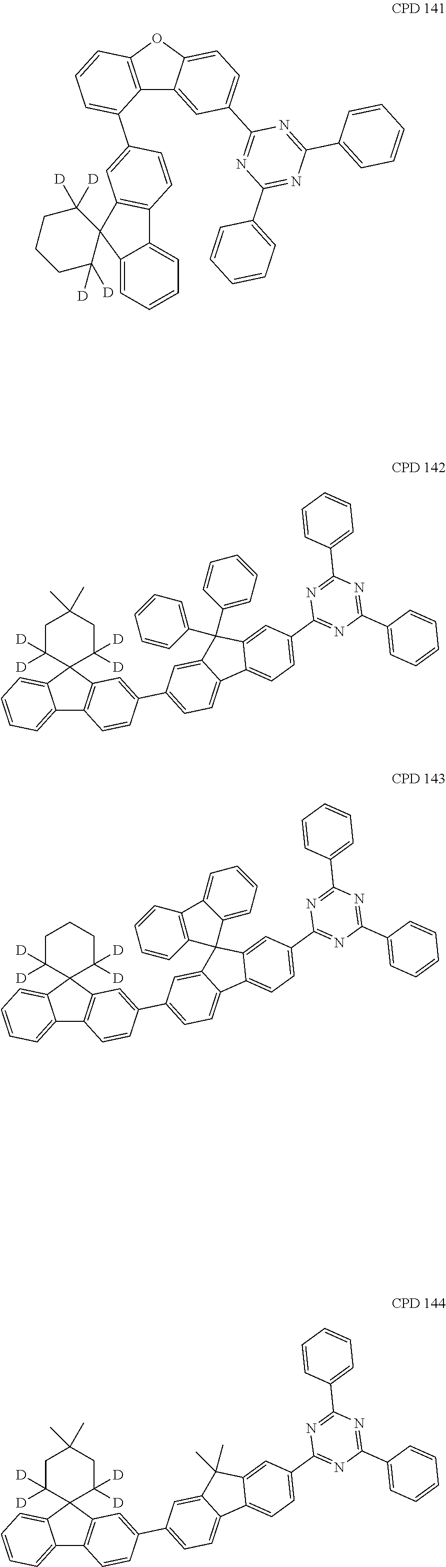 [SMILES: [2H]C1([2H])CCCC([2H])([2H])C12c1ccccc1-c1ccc(-c3cccc4oc5ccc(-c6nc(-c7ccccc7)nc(-c7ccccc7)n6)cc5c34)cc12 ; [2H]C1([2H])CC(C)(C)CC([2H])([2H])C12c1ccccc1-c1ccc(-c3ccc4c(c3)C(c3ccccc3)(c3ccccc3)c3cc(-c5nc(-c6ccccc6)nc(-c6ccccc6)n5)ccc3-4)cc12 ; [2H]C1([2H])CCCC([2H])([2H])C12c1ccccc1-c1ccc(-c3ccc4c(c3)C3(c5ccccc5-c5ccccc53)c3cc(-c5nc(-c6ccccc6)nc(-c6ccccc6)n5)ccc3-4)cc12 ; [2H]C1([2H])CC(C)(C)CC([2H])([2H])C12c1ccccc1-c1ccc(-c3ccc4c(c3)C(C)(C)c3cc(-c5nc(-c6ccccc6)nc(-c6ccccc6)n5)ccc3-4)cc12]

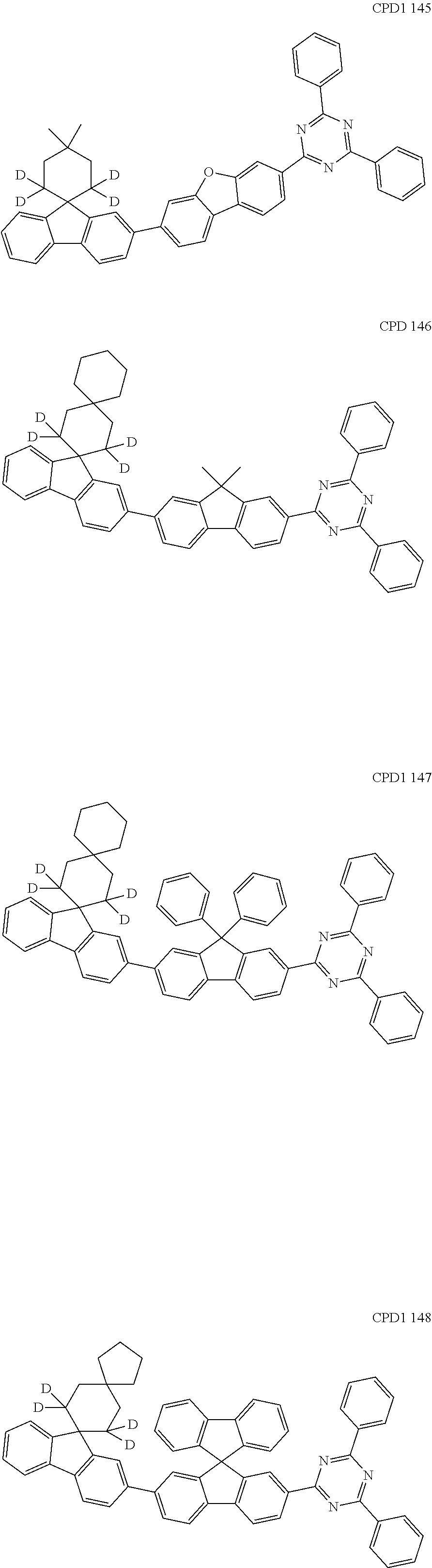

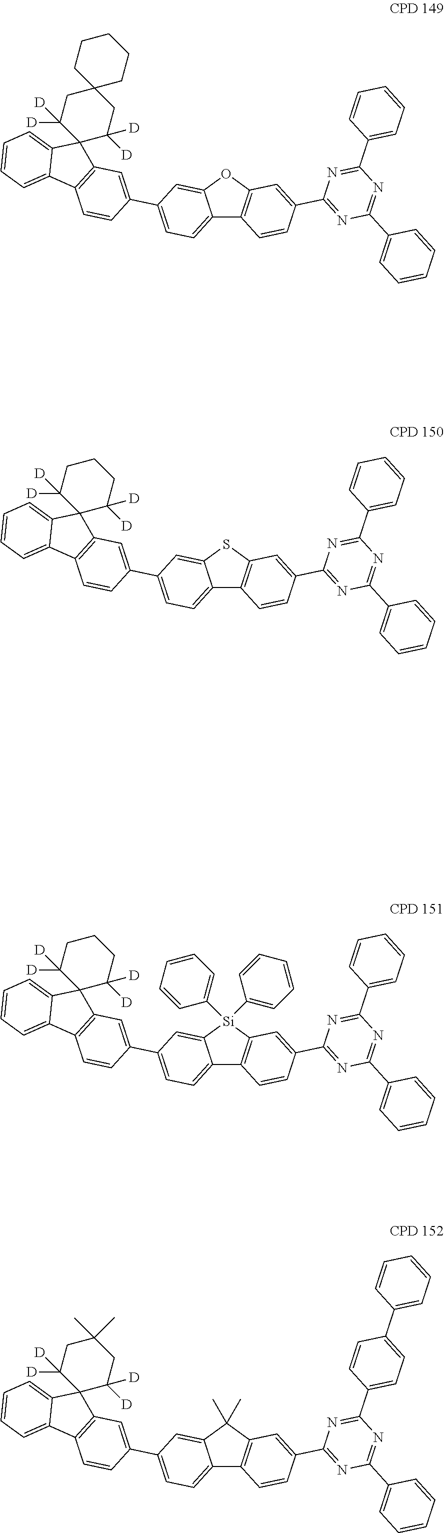 [SMILES: [2H]C1([2H])CC2(CCCCC2)CC([2H])([2H])C12c1ccccc1-c1ccc(-c3ccc4c(c3)oc3cc(-c5nc(-c6ccccc6)nc(-c6ccccc6)n5)ccc34)cc12 ; [2H]C1([2H])CCCC([2H])([2H])C12c1ccccc1-c1ccc(-c3ccc4c(c3)sc3cc(-c5nc(-c6ccccc6)nc(-c6ccccc6)n5)ccc34)cc12 ; [2H]C1([2H])CCCC([2H])([2H])C12c1ccccc1-c1ccc(-c3ccc4c(c3)[Si](c3ccccc3)(c3ccccc3)c3cc(-c5nc(-c6ccccc6)nc(-c6ccccc6)n5)ccc3-4)cc12 ; [2H]C1([2H])CC(C)(C)CC([2H])([2H])C12c1ccccc1-c1ccc(-c3ccc4c(c3)C(C)(C)c3cc(-c5nc(-c6ccccc6)nc(-c6ccc(-c7ccccc7)cc6)n5)ccc3-4)cc12]

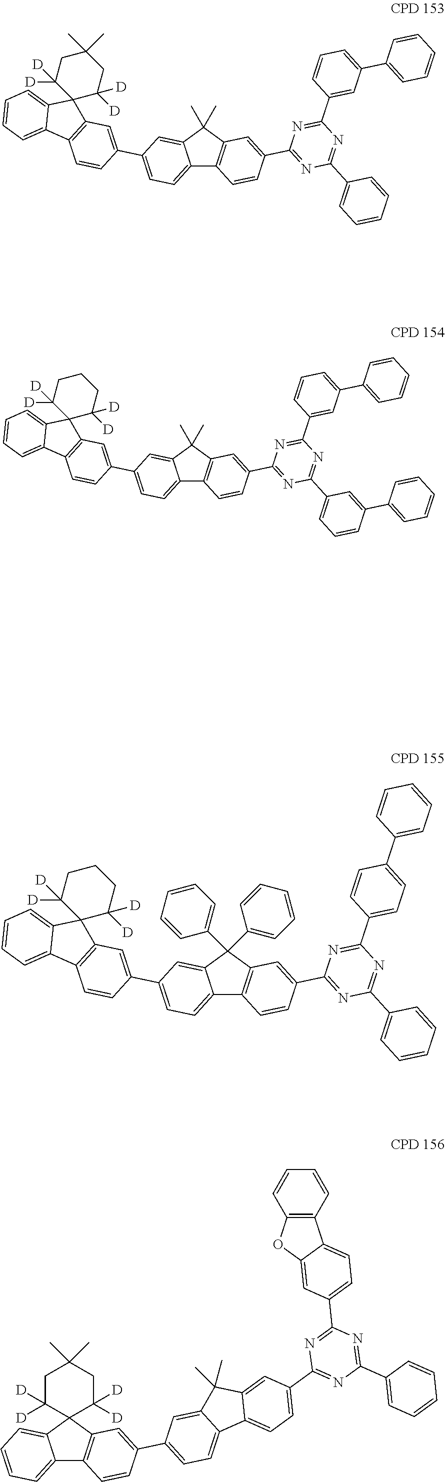

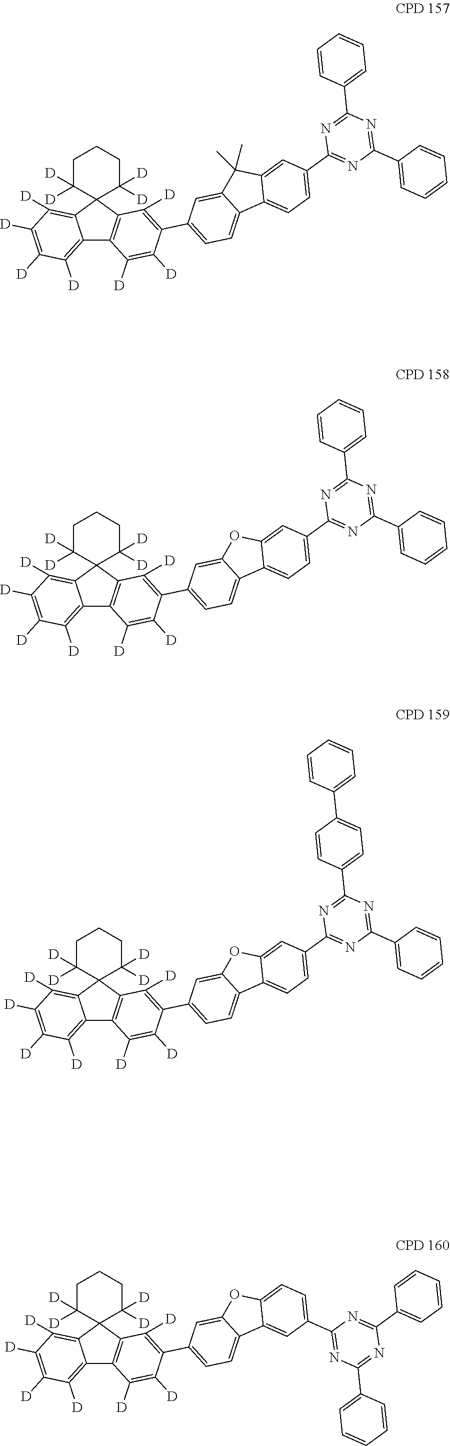

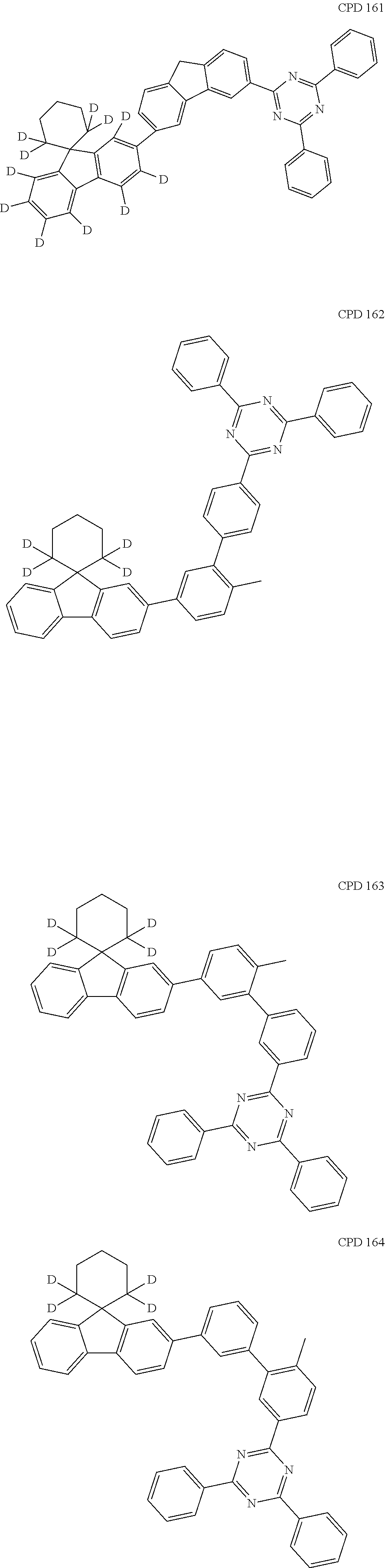 [SMILES: [2H]c1c([2H])c([2H])c2c(c1[2H])-c1c([2H])c([2H])c(-c3ccc4c(c3)-c3cc(-c5nc(-c6ccccc6)nc(-c6ccccc6)n5)ccc3C4)c([2H])c1C21C([2H])([2H])CCCC1([2H])[2H] ; [2H]C1([2H])CCCC([2H])([2H])C12c1ccccc1-c1ccc(-c3ccc(C)c(-c4ccc(-c5nc(-c6ccccc6)nc(-c6ccccc6)n5)cc4)c3)cc12 ; [2H]C1([2H])CCCC([2H])([2H])C12c1ccccc1-c1ccc(-c3ccc(C)c(-c4cccc(-c5nc(-c6ccccc6)nc(-c6ccccc6)n5)c4)c3)cc12 ; [2H]C1([2H])CCCC([2H])([2H])C12c1ccccc1-c1ccc(-c3cccc(-c4cc(-c5nc(-c6ccccc6)nc(-c6ccccc6)n5)ccc4C)c3)cc12]

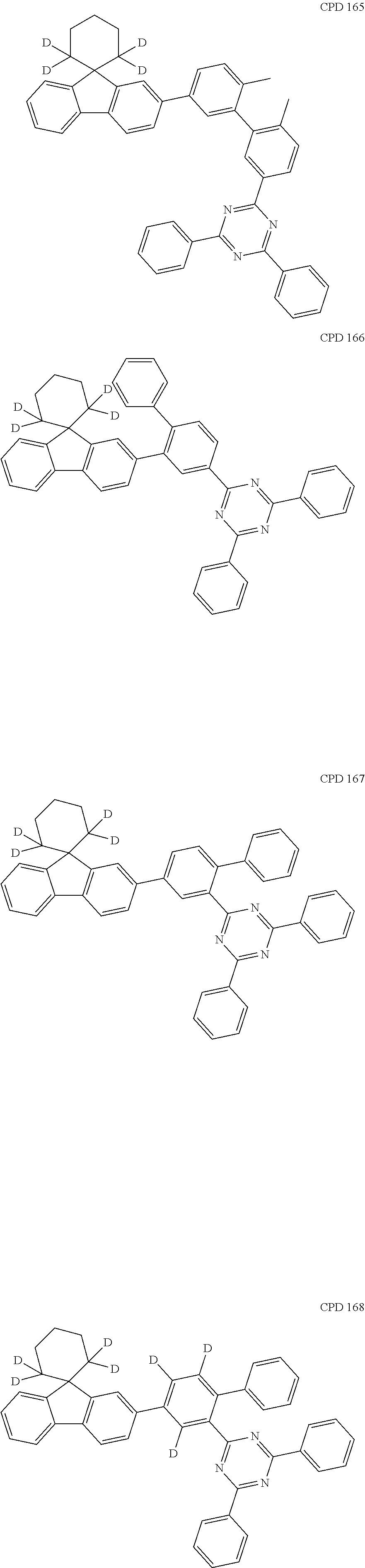 [SMILES: [2H]C1([2H])CCCC([2H])([2H])C12c1ccccc1-c1ccc(-c3ccc(C)c(-c4cc(-c5nc(-c6ccccc6)nc(-c6ccccc6)n5)ccc4C)c3)cc12 ; [2H]C1([2H])CCCC([2H])([2H])C12c1ccccc1-c1ccc(-c3cc(-c4nc(-c5ccccc5)nc(-c5ccccc5)n4)ccc3-c3ccccc3)cc12 ; [2H]C1([2H])CCCC([2H])([2H])C12c1ccccc1-c1ccc(-c3ccc(-c4ccccc4)c(-c4nc(-c5ccccc5)nc(-c5ccccc5)n4)c3)cc12 ; [2H]c1c([2H])c(-c2ccccc2)c(-c2nc(-c3ccccc3)nc(-c3ccccc3)n2)c([2H])c1-c1ccc2c(c1)C1(c3ccccc3-2)C([2H])([2H])CCCC1([2H])[2H]]

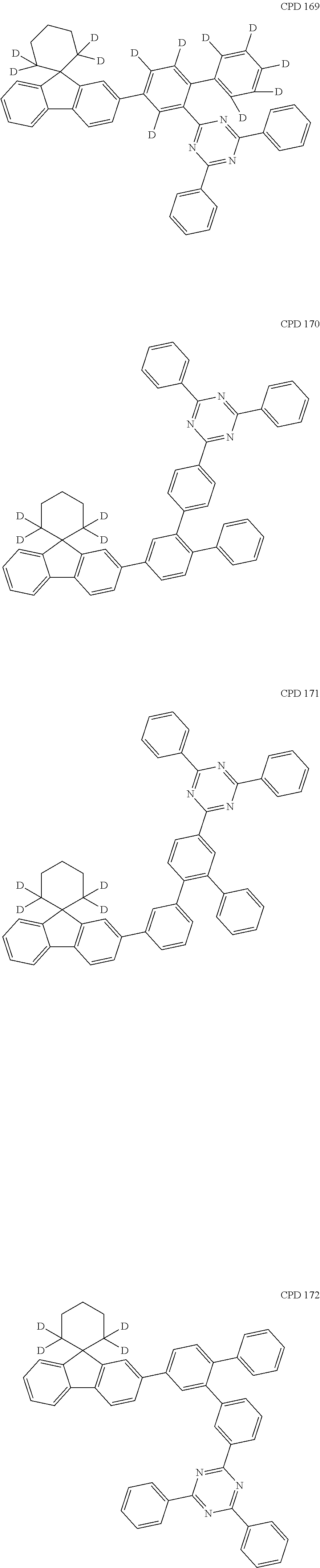

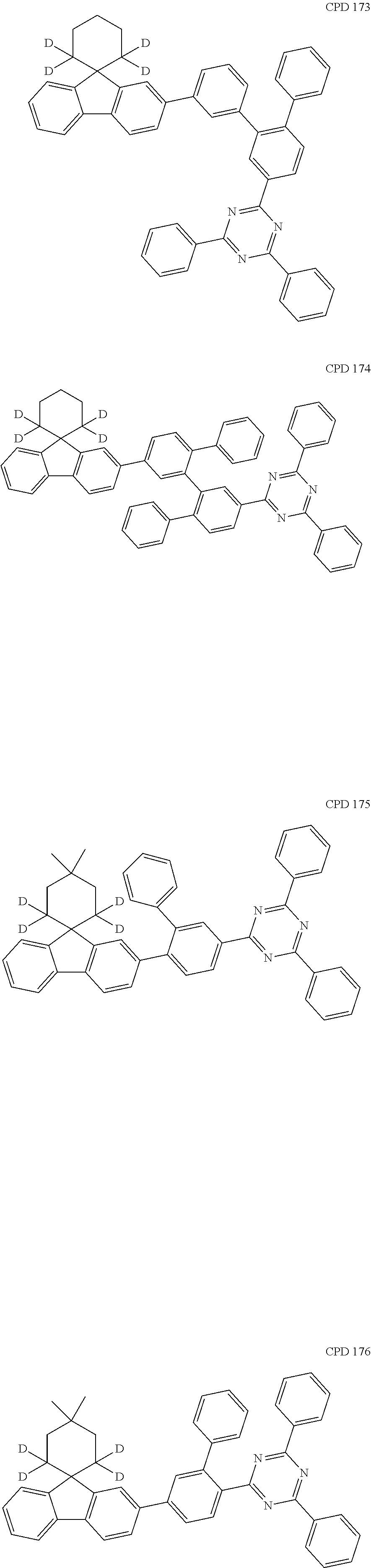 [SMILES: [2H]C1([2H])CCCC([2H])([2H])C12c1ccccc1-c1ccc(-c3cccc(-c4cc(-c5nc(-c6ccccc6)nc(-c6ccccc6)n5)ccc4-c4ccccc4)c3)cc12 ; [2H]C1([2H])CCCC([2H])([2H])C12c1ccccc1-c1ccc(-c3ccc(-c4ccccc4)c(-c4cc(-c5nc(-c6ccccc6)nc(-c6ccccc6)n5)ccc4-c4ccccc4)c3)cc12 ; [2H]C1([2H])CC(C)(C)CC([2H])([2H])C12c1ccccc1-c1ccc(-c3ccc(-c4nc(-c5ccccc5)nc(-c5ccccc5)n4)cc3-c3ccccc3)cc12 ; [2H]C1([2H])CC(C)(C)CC([2H])([2H])C12c1ccccc1-c1ccc(-c3ccc(-c4nc(-c5ccccc5)nc(-c5ccccc5)n4)c(-c4ccccc4)c3)cc12]

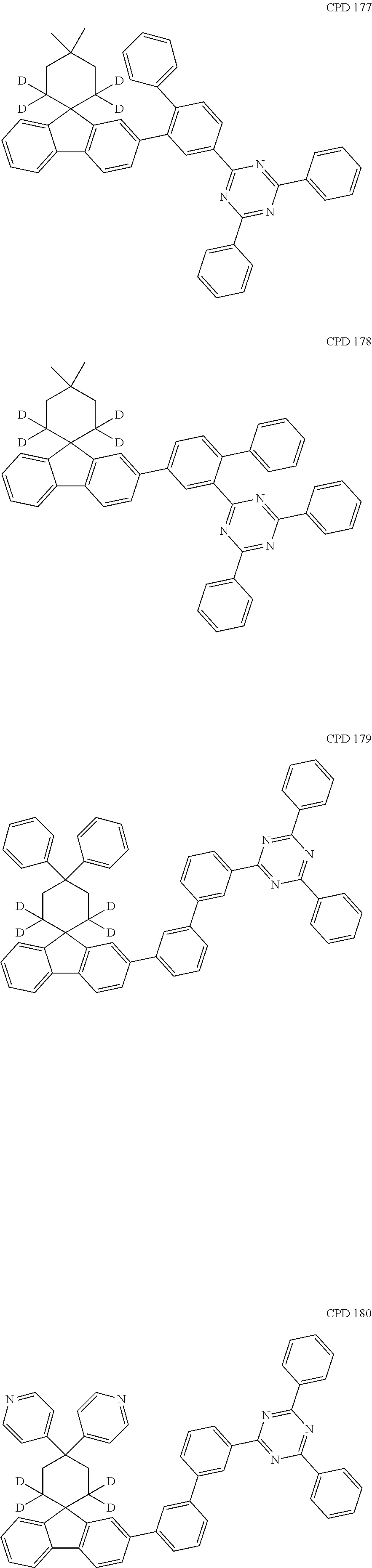 [SMILES: [2H]C1([2H])CC(C)(C)CC([2H])([2H])C12c1ccccc1-c1ccc(-c3cc(-c4nc(-c5ccccc5)nc(-c5ccccc5)n4)ccc3-c3ccccc3)cc12 ; [2H]C1([2H])CC(C)(C)CC([2H])([2H])C12c1ccccc1-c1ccc(-c3ccc(-c4ccccc4)c(-c4nc(-c5ccccc5)nc(-c5ccccc5)n4)c3)cc12 ; [2H]C1([2H])CC(c2ccccc2)(c2ccccc2)CC([2H])([2H])C12c1ccccc1-c1ccc(-c3cccc(-c4cccc(-c5nc(-c6ccccc6)nc(-c6ccccc6)n5)c4)c3)cc12 ; [2H]C1([2H])CC(c2ccncc2)(c2ccncc2)CC([2H])([2H])C12c1ccccc1-c1ccc(-c3cccc(-c4cccc(-c5nc(-c6ccccc6)nc(-c6ccccc6)n5)c4)c3)cc12]

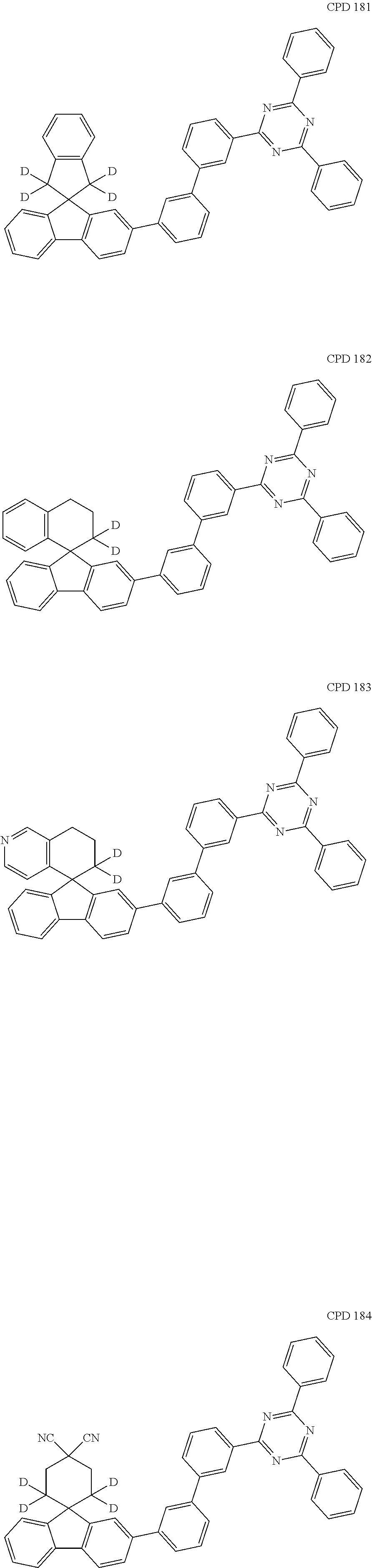 [SMILES: [2H]C1([2H])c2ccccc2C([2H])([2H])C12c1ccccc1-c1ccc(-c3cccc(-c4cccc(-c5nc(-c6ccccc6)nc(-c6ccccc6)n5)c4)c3)cc12 ; [2H]C1([2H])CCc2ccccc2C12c1ccccc1-c1ccc(-c3cccc(-c4cccc(-c5nc(-c6ccccc6)nc(-c6ccccc6)n5)c4)c3)cc12 ; [2H]C1([2H])CCc2cnccc2C12c1ccccc1-c1ccc(-c3cccc(-c4cccc(-c5nc(-c6ccccc6)nc(-c6ccccc6)n5)c4)c3)cc12 ; [2H]C1([2H])CC(C#N)(C#N)CC([2H])([2H])C12c1ccccc1-c1ccc(-c3cccc(-c4cccc(-c5nc(-c6ccccc6)nc(-c6ccccc6)n5)c4)c3)cc12]

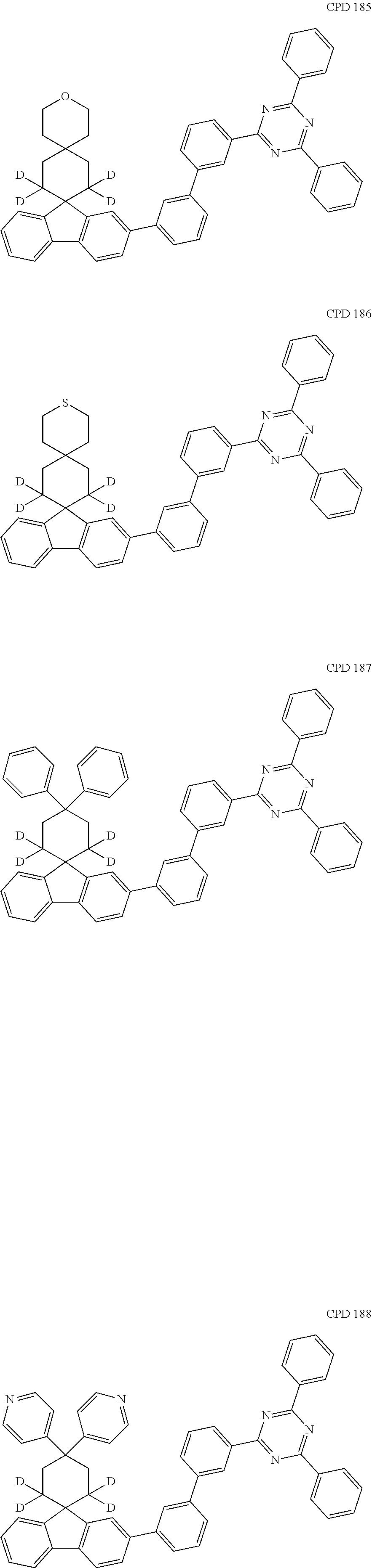 [SMILES: [2H]C1([2H])CC2(CCOCC2)CC([2H])([2H])C12c1ccccc1-c1ccc(-c3cccc(-c4cccc(-c5nc(-c6ccccc6)nc(-c6ccccc6)n5)c4)c3)cc12 ; [2H]C1([2H])CC2(CCSCC2)CC([2H])([2H])C12c1ccccc1-c1ccc(-c3cccc(-c4cccc(-c5nc(-c6ccccc6)nc(-c6ccccc6)n5)c4)c3)cc12 ; [2H]C1([2H])CC(c2ccccc2)(c2ccccc2)CC([2H])([2H])C12c1ccccc1-c1ccc(-c3cccc(-c4cccc(-c5nc(-c6ccccc6)nc(-c6ccccc6)n5)c4)c3)cc12 ; [2H]C1([2H])CC(c2ccncc2)(c2ccncc2)CC([2H])([2H])C12c1ccccc1-c1ccc(-c3cccc(-c4cccc(-c5nc(-c6ccccc6)nc(-c6ccccc6)n5)c4)c3)cc12]

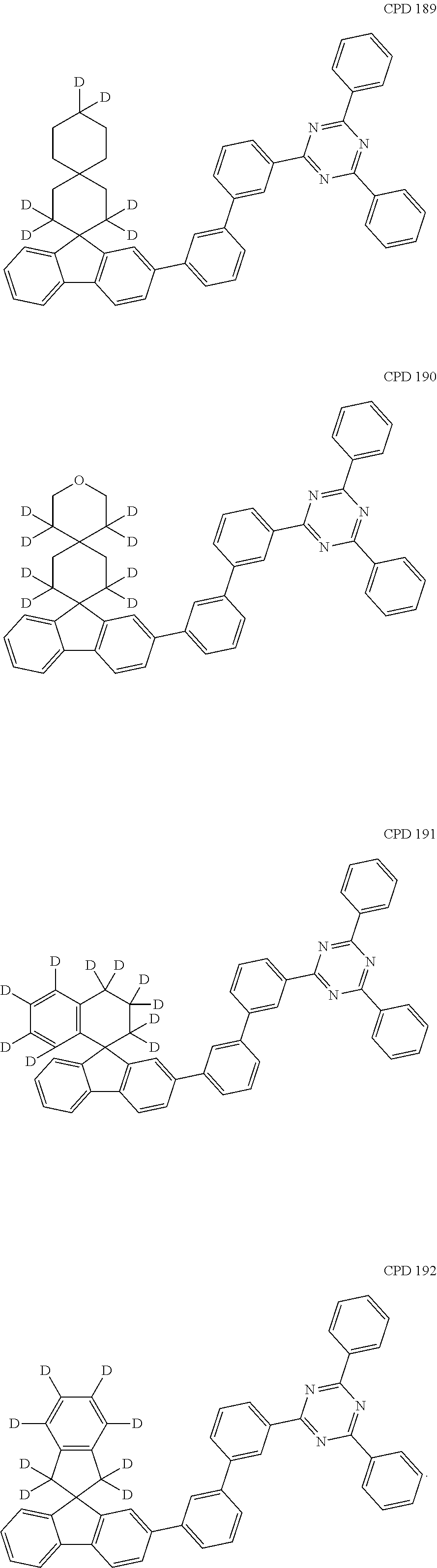

19. An electronic element comprising a cathode and an anode, wherein the cathode and the anode are arranged opposite to each other, and an intermediate layer is provided between the cathode and the anode; wherein the intermediate layer includes at least one layer of a hole blocking layer, an organic light-emitting layer, and an electron transport layer; and at least one layer of the intermediate layers contains the compound according to claim 1.

20. An electronic device comprising the electronic element according to claim 19.
